(19) United States
(12) Patent Application Publication
(10) Pub. No.: US 2023/0122967 A1
(43) Pub. Date: Apr. 20, 2023

(54) NOVEL COMPOUNDS AS INHIBITORS OF PCSK9

(71) Applicant: SHENGKE PHARMACEUTICALS (JIANGSU) LTD., Suzhou, Jiangsu (CN)

(72) Inventors: Soan Cheng, San Diego, CA (US); Chengzhi Yu, San Diego, CA (US); Zuopeng Sun, Shenyang (CN); Chenggang Zhang, Auckland (NZ)

(21) Appl. No.: 17/791,665

(22) PCT Filed: Jan. 14, 2021
(86) PCT No.: PCT/CN2021/071745

§ 371 Date: Jul. 8, 2022

(30) Foreign Application Priority Data
Jan. 17, 2020 (CN) PCT/CN2020/072748

(51) Int. Cl. C07D 209/42; C07D 307/85; C07D 307/68; C07D 209/14; C07D 233/90; C07D 263/34; C07D 261/10; C07D 307/52

(52) U.S. Cl. CPC C07D 209/42; C07D 307/85; C07D 307/68; C07D 209/14; C07D 233/90; C07D 263/34; C07D 261/10; C07D 307/52

(57) ABSTRACT

Disclosed are a compound of formula (I), wherein the variables are defined in the specification, a pharmaceutical composition containing the same, and a method and a use of the compound or composition in the treatment of a PCSK9-mediated disease such as cardiovascular disease.

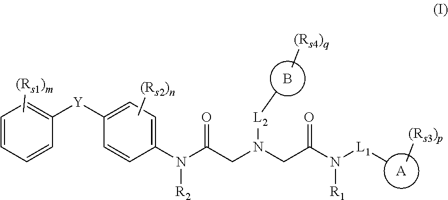

### BACKGROUND OF THE INVENTION

Cardiovascular diseases that occur in patients who have high levels of low-density lipoprotein cholesterol (LDL-C) are a leading cause of death in developed countries. Increased levels of LDL-C are considered a major risk factor for coronary artery disease (CAD) and for the development of atherosclerotic plaques in arteries. Cardiovascular risk is decreased when LDL-C is reduced.

Loss-of-function mutations in the low-density lipoprotein receptor (LDLR) gene in patients with familial hypercholesterolemia (FH) are associated with high plasma LDL-C levels and early-onset CAD, which begins in childhood. The LDLR, which is localized to the cell membrane, degrades the plasma LDL-C concentration via the receptor-mediated uptake of LDL-C into the cell.

One of the greatest advances in the lipid-lowering field over the past decade was the development of a lipid-altering therapy targeting proprotein convertase subtilisin/kexin type 9 (PCSK9), which binds to LDLRs and targets them for lysosomal degradation. This is due to the increase in the transcription of both PCSK9 and LDLR upon using statins, which leads to a decreased lipid restriction of statins.

Accordingly, it is expected that by suppressing the production of PCSK9 or inhibiting the function of PCSK9, the amount of the LDLR can be increased, and as a result, the blood LDL cholesterol level is thereby reduced.

From such point of view as described above, researches on inhibition of the function of PCSK9 or suppression of the production of the same are being conducted. For example, attempts of inhibiting the function with monoclonal antibodies directed to PCSK9, suppression of the production of PCSK9 by RNA interference, and the like have been reported. However, there is a need for the potent small molecular inhibitors to inhibit the function of PCSK9 for the patients with cardiovascular diseases.

### SUMMARY OF THE INVENTION

An object of the present invention is to provide a novel compound that has a blood LDL cholesterol-reducing action and is useful as an active ingredient of medicaments. More specifically, the present disclosure provides a method of downregulating the function of PCSK9.

In one aspect, the present disclosure relates to the compound of formula (I), or a pharmaceutically acceptable salt, an enantiomer, a diastereomer, a racemate, a solvate, a hydrate, a polymorph, a prodrug, or an isotope variant thereof, and mixtures thereof:

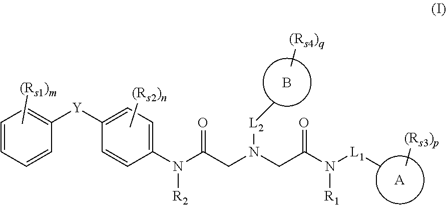

wherein:

Ring A is selected from C₃₋₇ cycloalkyl, 3- to 7-membered heterocyclyl, or C₆₋₁₀ aryl;

Ring B is selected from C₃₋₇ cycloalkyl, 3- to 7-membered heterocyclyl, C₆₋₁₀ aryl, or 5- to 10-membered heteroaryl;

L₁ is selected from a bond, —O—, —C(O)—, —CR′R″—, —CR′R″—CR′R″—, or —CR′R″—CR′R″—CR′R″—;

L₂ is selected from a bond, —C(O)—, —CR′R″—, —CR′R″—CR′R″—, or —CR′R″—CR′R″—CR′R″—;

Y is selected from O, S, NH, or CH₂;

R₁ is selected from H, —C(O)Rₐ, —C(O)ORₐ, —C(O)NR_{b}R_{c}, C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl;

R₂ is selected from H, C₁₋₆ alkyl, or C₁₋₆ haloalkyl;

Rₛ₁ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₂ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

m=0, 1, 2, 3, 4, or 5;

n=0, 1, 2, 3, or 4;

p=0, 1, 2, 3, 4, 5, 6, 7, or 8;

q=0, 1, 2, 3, 4, or 5;

and wherein,

R′ and R″ are each independently selected from H, halogen, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, or C₂₋₆ alkynyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

wherein each of Y, R₁, R₂, Rₛ₁, Rₛ₂, Rₛ₃, and Rₛ₄ is optionally substituted by 1, 2 or 3 R groups, wherein R is independently selected from H, —OH, halogen, —NO₂, carbonyl, -L-CN, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—NR_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, -L-O—C(S)—NR_{b}R_{c}, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl; wherein the said C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl is each further optionally substituted by one or more groups consisting of the following:

-L-CN, —NO₂, carbonyl, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—R_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, or -L-O—C(S)—NR_{b}R_{c};

L is selected from a chemical bond, —C₁₋₆ alkylene-, —C₂₋₆ alkenylene- or —C₂₋₆ alkynylene-;

x=0, 1 or 2.

In another aspect, the present disclosure provides a pharmaceutical composition comprising a compound of the present disclosure, and pharmaceutically acceptable excipients.

In another aspect, the present disclosure provides a pharmaceutical composition comprising a compound of the present disclosure and pharmaceutically acceptable excipients, which also includes other therapeutic agents, such as statins.

In another aspect, the present disclosure provides use of a compound of the present disclosure in the preparation of a medicament for the treatment and/or prevention of PCSK9-mediated diseases.

In another aspect, the present disclosure provides a method of treating and/or preventing PCSK9-mediated diseases in a subject, including administering a compound of the present disclosure or a composition of the present disclosure to the subject.

In another aspect, the present disclosure provides a compound or a composition of the present disclosure, for use in treating and/or preventing PCSK9-mediated diseases.

In a specific embodiment, the diseases described herein include atherosclerosis, dyslipidemia, hypertriglyceridemia, hypertension, heart failure, cardiac arrhythmias, low HDL levels, high LDL levels, sudden death, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia (including heterozygous and homozygous familial hypercholesterolemia), stroke, hyperlipidemia, hyperlipoproteinemia, chronic kidney disease, intermittent claudication, hyperphosphatemia, carotid atherosclerosis, peripheral arterial disease, diabetic nephropathy, hypercholesterolemia in HIV infection, acute coronary syndrome (ACS), non-alcoholic fatty liver disease, arterial occlusive diseases, cerebral arteriosclerosis, cerebrovascular disorders, myocardial ischemia, nonalcoholic fatty liver disease (NLLD), nonalcoholic steatohepatitis (NASH), and diabetic autonomic neuropathy.

Other objects and advantages of the present disclosure will be apparent to those skilled in the art from the subsequent specific embodiments, examples and claims.

### Definitions

Chemical Definitions

Definitions of specific functional groups and chemical terms are described in more detail hereafter.

When a range of values is listed, each value and sub-range within the range are intended to be included.

For example, “C₁₋₆ alkyl” is intended to include C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₆ alkyl” refers to a radical of a straight or branched, saturated hydrocarbon group having 1 to 6 carbon atoms. In some embodiments, C₁₋₄ alkyl is preferred. Examples of C₁₋₆ alkyl include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl, wherein one or more (e.g., 1, 2, 3 or 4) carbon atoms are substituted with heteroatoms (e.g., oxygen, sulfur, nitrogen, boron, silicon, phosphorus). Alkyl groups can be optionally substituted with one or more substituents, for example, with 1 to 5 substituents, 1 to 3 substituents or 1 substituent. Conventional abbreviations of alkyl include Me (—CH₃), Et (—CH₂CH₃), iPr (—CH(CH₃)₂), nPr (—CH₂CH₂CH₃), n-Bu (—CH₂CH₂CH₂CH₃) or i-Bu (—CH₂CH(CH₃)₂).

“C₂₋₆ alkenyl” refers to a radical of a straight or branched hydrocarbon group having 2 to 6 carbon atoms and at least one carbon-carbon double bond. In some embodiments, C₂₋₄ alkenyl is preferred. Examples of C₂₋₆ alkenyl include vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), etc. The term “C₂₋₆ alkenyl” also includes heteroalkenyl, wherein one or more (e.g., 1, 2, 3 or 4) carbon atoms are replaced by heteroatoms (e.g., oxygen, sulfur, nitrogen, boron, silicon, phosphorus). The alkenyl groups can be optionally substituted with one or more substituents, for example, with 1 to 5 substituents, 1 to 3 substituents or 1 substituent.

“C₂₋₆ alkynyl” refers to a radical of a straight or branched hydrocarbon group having 2 to 6 carbon atoms, at least one carbon-carbon triple bond and optionally one or more carbon-carbon double bonds. In some embodiments, C₂₋₄ alkynyl is preferred. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” also includes heteroalkynyl, wherein one or more (e.g., 1, 2, 3 or 4) carbon atoms are replaced by heteroatoms (e.g., oxygen, sulfur, nitrogen, boron, silicon, phosphorus). The alkynyl groups can be substituted with one or more substituents, for example, with 1 to 5 substituents, 1 to 3 substituents or 1 substituent.

“—C₁₋₆ alkylene-, —C₂₋₆ alkenylene- or —C₂₋₆ alkynylene-” refers to a divalent group of the “C₁₋₆ alkyl, C₂₋₆ alkenyl or C₂₋₆ alkynyl” as defined above.

“C₁₋₆ alkylene” refers to a divalent group formed by removing another hydrogen of the C₁₋₆ alkyl, and can be a substituted or unsubstituted alkylene. In some embodiments, C₁₋₄ alkylene is particularly preferred. The unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), etc. Examples of substituted alkylene groups, such as those substituted with one or more alkyl (methyl) groups, include, but are not limited to, substituted methylene (—CH(CH₃)—, —C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), etc.

“C₂₋₆ alkenylene” refers to a C₂₋₆ alkenyl group wherein another hydrogen is removed to provide a divalent radical of alkenylene, and which may be substituted or unsubstituted alkenylene. In some embodiments, C₂₋₄ alkenylene is particularly preferred. Exemplary unsubstituted alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“C₂₋₆ alkynylene” refers to a C₂₋₆ alkynyl group wherein another hydrogen is removed to provide a divalent radical of alkynylene, and which may be substituted or unsubstituted alkynylene. In some embodiments, C₂₋₄ alkynylene is particularly preferred. Exemplary alkynylene groups include, but are not limited to, ethynylene (—C≡C—), substituted or unsubstituted propynylene (—C≡CCH₂—), and the like.

“Halo” or “halogen” refers to fluorine (F), chlorine (Cl), bromine (Br) and iodine (I).

“C₁₋₆ haloalkyl” represents the “C₁₋₆ alkyl” described above, which is substituted with one or more halogen groups. Examples include the mono-, di-, poly-halogenated, including perhalogenated, alkyl. A monohalogen substituent may have one iodine, bromine, chlorine or fluorine atom in the group; a dihalogen substituent and a polyhalogen substituent may have two or more identical halogen atoms or a combination of different halogens. Examples of preferred haloalkyl groups include monofluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. The haloalkyl groups can be substituted at any available point of attachment, for example, with 1 to 5 substituents, 1 to 3 substituents or 1 substituent.

“C₃₋₇ cycloalkyl” refers to a radical of non-aromatic cyclic hydrocarbon group having 3 to 7 ring carbon atoms and zero heteroatoms. In some embodiments, C₃₋₆ cycloalkyl is particularly preferred, and C₅₋₆ cycloalkyl is more preferred. The cycloalkyl also includes a ring system in which the cycloalkyl described herein is fused with one or more aryl or heteroaryl groups, wherein the point of attachment is on the cycloalkyl ring, and in such case, the number of carbon atoms continues to represent the number of carbon atoms in the cycloalkyl system. Exemplary cycloalkyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl can be substituted with one or more substituents, for example, with 1 to 5 substituents, 1 to 3 substituents or 1 substituent.

“3- to 11-membered heterocyclyl” refers to a radical of 3- to 11-membered non-aromatic ring system having ring carbon atoms and 1 to 5 ring heteroatoms, wherein each of the heteroatoms is independently selected from nitrogen, oxygen, sulfur, boron, phosphorus and silicon. In the heterocyclyl containing one or more nitrogen atoms, the point of attachment can be a carbon or nitrogen atom as long as the valence permits. In some embodiments, 3- to 9-membered heterocyclyl is preferred, which is a radical of 3- to 9-membered non-aromatic ring system having ring carbon atoms and 1 to 5 ring heteroatoms. In some embodiments, 3- to 7-membered heterocyclyl is preferred, which is a radical of 3- to 7-membered non-aromatic ring system having ring carbon atoms and 1 to 4 ring heteroatoms. 3- to 6-membered heterocyclyl is preferred, which is a radical of 3- to 6-membered non-aromatic ring system having ring carbon atoms and 1 to 3 ring heteroatoms. 4- to 6-membered heterocyclyl is preferred, which is a radical of 4- to 6-membered non-aromatic ring system having ring carbon atoms and 1 to 3 ring heteroatoms. 5- to 6-membered heterocyclyl is more preferred, which is a radical of 5- to 6-membered non-aromatic ring system having ring carbon atoms and 1 to 3 ring heteroatoms. The heterocyclyl also includes a ring system wherein the heterocyclyl described above is fused with one or more cycloalkyl groups, wherein the point of attachment is on the cycloalkyl ring, or the heterocyclyl described above is fused with one or more aryl or heteroaryl groups, wherein the point of attachment is on the heterocyclyl ring; and in such cases, the number of ring members continues to represent the number of ring members in the heterocyclyl ring system. Exemplary 3-membered heterocyclyl groups containing one heteroatom include, but are not limited to, aziridinyl, oxiranyl and thiorenyl. Exemplary 4-membered heterocyclyl groups containing one heteroatom include, but are not limited to, azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl and pyrrolyl-2,5-dione. Exemplary 5-membered heterocyclyl groups containing two heteroatoms include, but are not limited to, dioxolanyl, oxasulfuranyl, disulfuranyl, and oxazolidin-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridyl and thianyl. Exemplary 6-membered heterocyclyl groups containing two heteroatoms include, but are not limited to, piperazinyl, morpholinyl, dithianyl and dioxanyl. Exemplary 6-membered heterocyclyl groups containing three heteroatoms include, but are not limited to, triazinanyl. Exemplary 7-membered heterocycly groups containing one heteroatom include, but are not limited to, azepanyl, oxepanyl and thiepanyl. Exemplary 5-membered heterocyclyl groups fused with a C₆ aryl (also referred as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused with a C₆ aryl (also referred as 6,6-bicyclic heterocyclyl herein) include, but are not limited to, tetrahydroquinolinyl, tetrahydroisoquinolinyl, etc. The heterocyclyl can be substituted with one or more substituents, for example, with 1 to 5 substituents, 1 to 3 substituents or 1 substituent.

The 3- to 11-membered heterocyclyl also includes spiroheterocyclyl, that is, a group in which two rings (e.g., a heterocycle and a carbocycle) share a carbon atom, wherein at least one of the rings is a heterocyclyl as defined above. More specifically, the spiroheterocyclyl is a spiro ring formed by two 4-membered rings, two 5-membered rings, two 6-membered rings, one 4-membered ring and one 5-membered ring, one 4-membered ring and one 6-membered ring, or one 5-membered ring and one 6-membered ring, wherein at least one of the rings is a 4- to 6-membered heterocyclyl as defined above. The 4- to 6-membered heterocyclyl containing 1,2 or 3 O, N or S heteroatoms is preferred.

Specific examples of preferred heterocyclyl groups include, pyrrolinyl, imidazolidinyl, pyrazolidinyl, tetrahydropyranyl, dihydropyranyl, dihydrofuranyl, thiazolidinyl, dihydrothiazolyl, 2,3-dihydro-benzo[1,4]dioxol, indolinyl, isoindolinyl, dihydrobenzothiophene, dihydrobenzofuranyl, isodihydrobenzopyranyl, dihydrobenzopyranyl, 1,2-dihydroisoquinoline, 1,2,3,4-tetrahydroisoquinoline, 1,2,3,4-tetrahydroquinoline, 2,3,4,4a,9,9a-hexahydro-1H-3-azafluorene, 5,6,7-trihydro-1,2,4-triazolo[3,4-a]isoquinolyl, 3,4-dihydro-2H-benzo[1,4]oxazinyl, benzo[1,4]dioxol, 2,3-dihydro-1H-1k′-benzo[d]isothiazol-6-yl, 2,3-di-benzo[1,4]dioxinyl, dihydrobenzofuran, 2-oxoaziridin-1-yl, 2-oxoazetidin-1-yl, 2-oxopyrrolidin-1-yl, 2-oxopiperidin-1-yl, 2-oxoazepan-1-yl, 2-oxoazocan-1-yl, 2-oxoazonan-1-yl, 2-oxoazecan-1-yl, aziridine, azetidine, pyrrolidinyl, piperidine, azepane, azocane, azonane, azecane, piperidyl, piperazinyl, morpholinyl, diazaspiro[3.3]heptane, diazaspiro[3.4]octane, diazaspiro[3.5]nonane, diazaspiro[4.4]nonane, diazaspiro[4.5]decane, and diazaspiro[5.5]undecane.

“C₆₋₁₀ aryl” refers to a radical of monocyclic or polycyclic (e.g., bicyclic) 4n+2 aromatic ring system having 6-10 ring carbon atoms and zero heteroatoms (e.g., having 6 or 10 shared π electrons in a cyclic array). In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl can be substituted with one or more substituents, for example, with 1 to 5 substituents, 1 to 3 substituents or 1 substituent.

“5- to 10-membered heteroaryl” refers to a radical of 5- to 10-membered monocyclic or bicyclic 4n+2 aromatic ring system (e.g., having 6 or 10 shared π electrons in a cyclic array) having ring carbon atoms and 1-4 ring heteroatoms, wherein each heteroatom is independently selected from nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number the carbon atoms continues to represent the number of carbon atoms in the heteroaryl ring system. In some embodiments, 5- to 6-membered heteroaryl groups are particularly preferred, which are radicals of 5- to 6-membered monocyclic or bicyclic 4n+2 aromatic ring systems having ring carbon atoms and 1-4 ring heteroatoms. Exemplary 5-membered heteroaryl groups containing one heteroatom include, but are not limited to, pyrrolyl, furyl and thienyl. Exemplary 5-membered heteroaryl groups containing two heteroatoms include, but are not limited to, imidazolyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazoly), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl, respectively. Exemplary 7-membered heteroaryl groups containing one heteroatom include, but are not limited to, azepinyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

Specific examples of preferred heteroaryl groups include: pyrrolyl, imidazolyl, pyrazolyl, 2-pyridyl, 3-pyridyl, 4-pyridyl, pyrimidinyl, pyrazinyl, pyridazinyl, triazolyl (4H-1,2,4-triazolyl, 1H-1,2,3-triazolyl, 2H-1,2,3-triazolyl, pyranyl, 2-furyl, 3-furyl, 2-thienyl, 3-thienyl, oxazolyl, isoxazolyl, oxazolyl (1,2,4-oxazolyl, 1,3,4-oxazolyl, 1,2,5-oxazolyl, thiazolyl, thiadiazolyl (1,2,4-thiadiazolyl, 1,3,4-thiadiazolyl, 1,2,5-thiadiazolyl).

“carbonyl”, whether used alone or in conjunction with other terms (e.g., aminocarbonyl), is represented by —C(O)—.

Alkyl, alkenyl, alkynyl, carbocyclyl, heterocyclyl, aryl and heteroaryl as defined herein are optionally substituted groups.

Exemplary substituents on carbon atoms include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR—, —SSR^{cc}, —C(═O)R—, —CO₂H, —CHO, —C(OR^{cc})₂, —CO₂R—, —OC(═O)R″, —OCO₂Rₐ^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R″, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR—, —SC(═O)SR^{aa}, —OC(═O)SR—, —SC(═O)OR—, —SC(═O)R^{aa}, —P(═O)₂R^{aa}, —OP(═O)₂R^{aa}, —P(═O)(R^{a})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, carbocyclyl, heterocyclyl, aryl and heteroaryl, wherein each of the alkyl, alkenyl, alkynyl, carbocyclyl, heterocyclyl, aryl and heteroaryl is independently substituted with 0, 1, 2, 3, 4 or 5 R^{dd} groups;

or two geminal hydrogen on a carbon atom are replaced with ═O, ═S, ═NN(R^{bb})₂, ═NNR^{bb}C(═O)R—, ═NNR^{bb}C(═O)OR^{aa}, ═NNR^{bb}S(═O)₂R^{aa}, ═NR^{bb} or ═NOR^{cc} groups;

each of the R^{aa} is independently selected from alkyl, haloalkyl, alkenyl, alkynyl, carbocyclyl, heterocyclyl, aryl and heteroaryl, or two of the R^{aa} groups are combined to form a heterocyclyl or heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups;

each of the R^{bb} is independently selected from hydrogen, —OH, —OR^{aa}, —N(R^{CC})₂, —CN, —C(═O)R^{aa}, —C(═O)N(R^{cc})₂, —CO₂R^{aa}, —SO₂R^{aa}, —C(═NR^{cc})OR^{a}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{aa}, —C(═S)SR^{aa}, —P(═O)₂R^{aa}, —P(═O)(R″)₂, —P(═O)₂N(R^{cc})₂, —P(═O)(NR^{cc})₂, alkyl, haloalkyl, alkenyl, alkynyl, carbocyclyl, heterocyclyl, aryl and heteroaryl, or two R^{bb} groups are combined to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups;

each of the R^{cc} is independently selected from hydrogen, alkyl, haloalkyl, alkenyl, alkynyl, carbocyclyl, heterocyclyl, aryl and heteroaryl, or two R^{cc} groups are combined to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups;

each of the R^{dd} is independently selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{aa}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, carbocyclyl, heterocyclyl, aryl, heteroaryl, wherein each of the alkyl, alkenyl, alkynyl, carbocyclyl, heterocyclyl, aryl and heteroaryl is independently substituted with 0, 1, 2, 3, 4 or 5 R^{gg} groups, or two geminal R^{dd} substituents can be combined to form ═O or ═S;

each of the R^{ee} is independently selected from alkyl, haloalkyl, alkenyl, alkynyl, carbocyclyl, aryl, heterocyclyl, and heteroaryl, wherein each of the alkyl, alkenyl, alkynyl, carbocyclyl, heterocyclyl, aryl and heteroaryl is independently substituted with 0, 1, 2, 3, 4 or 5 R^{gg} groups;

each of the R^{f} is independently selected from hydrogen, alkyl, haloalkyl, alkenyl, alkynyl, carbocyclyl, heterocyclyl, aryl and heteroaryl, or two R^{ff} groups are combined to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{gg} groups;

each of the R^{gg} is independently selected from halogen, —CN, —NO₂, —N₃, —SO₂H, —SO₃H, —OH, —OC₁₋₆alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ carbocyclyl, C₆-C₁₀ aryl, C₃-C₇ heterocyclyl, C₅-C₁₀ heteroaryl; or two geminal R^{gg} substituents may combine to form ═O or ═S; wherein X⁻ is a counter-ion.

Exemplary substituents on nitrogen atoms include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, carbocyclyl, heterocyclyl, aryl and heteroaryl, or two R^{cc} groups attached to a nitrogen atom combine to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

### Other Definitions

The term “treating” as used herein relates to reversing, alleviating or inhibiting the progression or prevention of the disorders or conditions to which the term applies, or of one or more symptoms of such disorders or conditions. The noun “treatment” as used herein relates to the action of treating, which is a verb, and the latter is as just defined.

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

The term “salt” refers to a relatively non-toxic addition salt of inorganic and organic acids to the compounds of the present disclosure. These salts can be prepared in situ during the final separation and purification of the compounds, or by isolating salts produced by separately reacting the purified compound in the free base form with a suitable organic or inorganic acid. As long as the compounds of the present disclosure are basic compounds, they are capable of forming a plurality of different salts with various inorganic and organic acids. Although such salts must be pharmaceutically acceptable for animal administration, it is often necessary in practice that the pharmaceutically unacceptable salts of the basic compounds are first isolated from the reaction mixture, and then they are simply treated with an alkaline agent to convert to the free base compound, followed by the conversion of the free base to pharmaceutically acceptable acid addition salts. The acid addition salts of the basic compounds are prepared by contacting the free base form with a sufficient amount of the acid required in a conventional manner to form the salts. The free base can be regenerated by contacting the salt form with the base in a conventional manner and then isolating the free base. The free base forms are somewhat different from their respective salt forms in some physical properties, such as solubility in polar solvents. But for the purposes of the present disclosure, the salts are still equivalent to their respective free bases.

The pharmaceutically acceptable base addition salts are formed with metals or amines, such as alkali metal and alkaline earth metal hydroxides or organic amines. Examples of the metals used as cations include sodium, potassium, magnesium, calcium, etc. Examples of suitable amines are N,N′-dibenzylethylenediamine, chloroprocaine, choline, diethanolamine, ethylenediamine, N-methylglucamine and procaine.

The base addition salt of the acidic compound can be prepared by contacting the free acid form with a sufficient amount of the required base to form a salt in a conventional manner. The free acid can be regenerated by contacting the salt form with an acid in a conventional manner and then isolating the free acid. The free acid forms are somewhat different from their respective salt forms in their physical properties, such as solubility in polar solvents. But for the purposes of the present disclosure, the salts are still equivalent to their respective free acids.

The salts can be prepared from the inorganic acids, which include sulfates, pyrosulfates, bisulfates, sulfites, bisulfites, nitrates, phosphates, monohydrogen phosphates, dihydrogen phosphates, metaphosphates, pyrophosphates, chlorides, bromides and iodides. Examples of the acids include hydrochloric acid, nitric acid, sulfuric acid, hydrobromic acid, hydroiodic acid, phosphoric acid, etc. The representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthalate, methanesulfonate, glucoheptanate, lactobionate, lauryl sulfonate, isethionate, etc. The salts can also be prepared from the organic acids, which include aliphatic monocarboxylic and dicarboxylic acids, phenyl-substituted alkanoic acids, hydroxyalkanoic acids, alkanedioic acid, aromatic acids, aliphatic and aromatic sulfonic acids, etc. The representative salts include acetate, propionate, octanoate, isobutyrate, oxalate, malonate, succinate, suberate, sebacate, fumarate, maleate, mandelate, benzoate, chlorobenzoate, methyl benzoate, dinitrobenzoate, naphthoate, besylate, tosylate, phenylacetate, citrate, lactate, maleate, tartrate, methanesulfonate, etc. The pharmaceutically acceptable salts can include cations based on alkali metals and alkaline earth metals, such as sodium, lithium, potassium, calcium, magnesium, etc., as well as non-toxic ammonium, quaternary ammonium, and amine cations including, but not limited to, ammonium, tetramethylammonium, tetraethylammonium, methylamine, dimethylamine, trimethylamine, triethylamine, ethylamine, etc. Salts of amino acids are also included, such as arginine salts, gluconates, galacturonates, etc. (for example, see Berge S. M. et al., “Pharmaceutical Salts,” J. Pharm. Sci., 1977; 66: 1-19 for reference).

Examples of pharmaceutically acceptable non-toxic amides of the compounds of the disclosure include C₁-C₆ alkyl esters, wherein the alkyl group is straight or branched. Acceptable esters also include C₅-C₇ cycloalkyl esters as well as arylalkyl esters, such as, but not limited to, benzyl esters. C₁-C₄ alkyl esters are preferred. Esters of the compounds of the disclosure can be prepared according to the conventional methods, for example, March's Advanced Organic Chemistry, 5 Edition, M. B. Smith & J. March, John Wiley & Sons, 2001.

Examples of pharmaceutically acceptable non-toxic amides of the compounds of the disclosure include amides derived from ammonia, primary C₁-C₆alkylamines and secondary C₁-C₆ dialkylamines, wherein the alkyl group is straight or branched. In the case of the secondary amine, the amine may also be in the form of a 5- or 6-membered heterocycle containing one nitrogen atom. Amides derived from ammonia, C₁-C₃ alkyl primary amine and C₁-C₂ dialkyl secondary amine are preferred. Amides of the compounds of the present disclosure can be prepared according to the conventional methods, for example, March's Advanced Organic Chemistry, 5 Edition, M. B. Smith & J. March, John Wiley & Sons, 2001.

“Subjects” to which administration is contemplated include, but are not limited to, humans (e.g., males or females of any age group, e.g., paediatric subjects (e.g., infants, children, adolescents) or adult subjects (e.g., young adults, middle-aged adults or older adults) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “human”, “patient” and “subject” can be used interchangeably herein.

“Disease,” “disorder,” and “condition” can be used interchangeably herein.

Unless indicated, otherwise the term “treatment” as used herein includes the effect on a subject who is suffering from a particular disease, disorder, or condition, which reduces the severity of the disease, disorder, or condition, or delays or slows the progression of the disease, disorder or condition (“therapeutic treatment”). The term also includes the effect that occurs before the subject begins to suffer from a specific disease, disorder or condition (“prophylactic treatment”).

Generally, the “effective amount” of a compound refers to an amount sufficient to elicit a target biological response. As understood by those skilled in the art, the effective amount of the compound of the disclosure can vary depending on the following factors, such as the desired biological endpoint, the pharmacokinetics of the compound, the diseases being treated, the mode of administration, and the age, health status and symptoms of the subjects. The effective amount includes therapeutically effective amount and prophylactically effective amount.

Unless indicated, otherwise the “therapeutically effective amount” of the compound as used herein is an amount sufficient to provide therapeutic benefits in the course of treating a disease, disorder or condition, or to delay or minimize one or more symptoms associated with the disease, disorder or condition. The therapeutically effective amount of a compound refers to the amount of the therapeutic agent that, when used alone or in combination with other therapies, provides a therapeutic benefit in the treatment of a disease, disorder or condition. The term “therapeutically effective amount” can include an amount that improves the overall treatment, reduces or avoids the symptoms or causes of the disease or condition, or enhances the therapeutic effect of other therapeutic agents.

Unless indicated, otherwise the “prophylactically effective amount” of the compound as used herein is an amount sufficient to prevent a disease, disorder or condition, or an amount sufficient to prevent one or more symptoms associated with a disease, disorder or condition, or an amount sufficient to prevent the recurrence of a disease, disorder or condition. The prophylactically effective amount of a compound refers to the amount of a therapeutic agent that, when used alone or in combination with other agents, provides a prophylactic benefit in the prevention of a disease, disorder or condition. The term “prophylactically effective amount” can include an amount that improves the overall prevention, or an amount that enhances the prophylactic effect of other preventive agents.

“Combination” and related terms refer to the simultaneous or sequential administration of the compounds of the present disclosure and other therapeutic agents. For example, the compounds of the present disclosure can be administered simultaneously or sequentially in separate unit dosage with other therapeutic agents, or simultaneously in a single unit dosage with other therapeutic agents.

### DETAILED DESCRIPTION OF THE INVENTION

As used herein, “compounds of the present disclosure” refer to the compounds of formula (I), formula (II-1), and the like as shown below, or pharmaceutically acceptable salts, enantiomers, diastereomers, racemates, solvates, hydrates, polymorphs, prodrugs or isotope variants thereof, and mixtures thereof.

Compounds are generally described herein using standard nomenclature. It should be understood, unless otherwise specified, that compounds with asymmetric center(s) include all optical isomers and mixtures thereof. Furthermore, unless otherwise specified, all isomer compounds and carbon-carbon double bonds included in the present disclosure may be in the form of Z and E. Compounds which exist in different tautomeric forms, one of which is not limited to any particular tautomer, but is intended to cover all tautomeric forms.

In one embodiment, the present disclosure refers to a compound of formula (I), or a pharmaceutically acceptable salt, an enantiomer, a diastereomer, a racemate, a solvate, a hydrate, a polymorph, a prodrug, or an isotope variant thereof, and mixtures thereof:

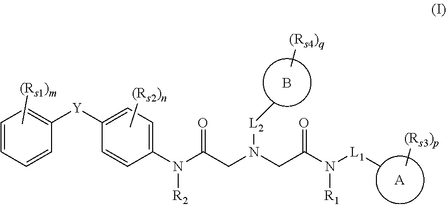

wherein:

Ring A is selected from C₃₋₇ cycloalkyl, 3- to 7-membered heterocyclyl, or C₆₋₁₀ aryl;

Ring B is selected from C₃₋₇ cycloalkyl, 3- to 7-membered heterocyclyl, C₆₋₁₀ aryl, or 5- to 10-membered heteroaryl;

L₁ is selected from a bond, —O—, —C(O)—, —CR′R″—, —CR′R″—CR′R″—, or —CR′R″—CR′R″—CR′R″—;

L₂ is selected from a bond, —C(O)—, —CR′R″—, —CR′R″—CR′R″—, or —CR′R″—CR′R″—CR′R″—;

Y is selected from O, S, NH, or CH₂;

R₁ is selected from H, —C(O)Rₐ, —C(O)ORₐ, —C(O)NR_{b}R_{c}, C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl;

R₂ is selected from H, C₁₋₆ alkyl, or C₁₋₆ haloalkyl;

Rₛ₁ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₂ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂ 6 alkynyl, C₃₋₇ cycloalkyl, 3- to 7-membered heterocyclyl, C₆₋₁₀ aryl, or 5- to 10-membered heteroaryl;

Rₛ₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

m=0, 1, 2, 3, 4, or 5;

n=0, 1, 2, 3, or 4;

p=0, 1, 2, 3, 4, 5, 6, 7, or 8;

q=0, 1, 2, 3, 4, or 5;

and wherein,

R′ and R″ are each independently selected from H, halogen, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, or C₂₋₆ alkynyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

wherein each of Y, R₁, R₂, Rₛ₁, Rₛ₂, Rₛ₃, and Rₛ₄ is optionally substituted by 1, 2 or 3 R groups, wherein R is independently selected from H, —OH, halogen, —NO₂, carbonyl, -L-CN, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—NR_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, -L-O—C(S)—NR_{b}R_{c}, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl; wherein the said C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl is each further optionally substituted by one or more groups consisting of the following:

-L-CN, —NO₂, carbonyl, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—R_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, or -L-O—C(S)—NR_{b}R_{c};

L is selected from a chemical bond, —C₁₋₆ alkylene-, —C₂₋₆ alkenylene- or —C₂₋₆ alkynylene-;

x=0, 1 or 2.

Ring A

In one embodiment, Ring A is C₃₋₇ cycloalkyl; in another embodiment, Ring A is 3- to 7-membered heterocyclyl; in another embodiment, Ring A is C₆₋₁₀ aryl.

Ring B

In one embodiment, Ring B is C₃₋₇ cycloalkyl; in another embodiment, Ring B is 3- to 7-membered heterocyclyl; in another embodiment, Ring B is C₆₋₁₀ aryl; in another embodiment, Ring B is 5- to 10-membered heteroaryl.

L₁

In one embodiment, L₁ is a bond; in another embodiment, L₁ is —O—; in another embodiment, L₁ is —C(O)—; in another embodiment, L₁ is —CR′R″—; in another embodiment, L₁ is —CR′R″—CR′R″—; in another embodiment, L₁ is —CR′R″—CR′R″—CR′R″—.

L₂

In one embodiment, L₂ is a bond; in another embodiment, L₂ is —C(O)—; in another embodiment, L₂ is —CR′R″—; in another embodiment, L₂ is —CR′R″—CR′R″—; in another embodiment, L₂ is —CR′R″—CR′R″—CR′R″—.

Y

In one embodiment, Y is O; in another embodiment, Y is S; in another embodiment, Y is NH; in another embodiment, Y is CH₂.

R₁

In one embodiment, R₁ is H; in another embodiment, R₁ is —C(O)Rₐ; in another embodiment, R₁ is —C(O)ORₐ; in another embodiment, R₁ is —C(O)NR_{b}R_{c}; in another embodiment, R₁ is C₁₋₆ alkyl; in another embodiment, R₁ is C₁₋₆ haloalkyl; in another embodiment, R₁ is C₃₋₇ cycloalkyl; in another embodiment, R₁ is 3- to 7-membered heterocyclyl; in another embodiment, R₁ is C₆₋₁₀ aryl; in another embodiment, R₁ is 5- to 10-membered heteroaryl.

R₂

In one embodiment, R₂ is H; in another embodiment, R₂ is C₁₋₆ alkyl; in another embodiment, R₂ is C₁₋₆ haloalkyl.

Rₛ₁

In one embodiment, R₁ is H; in another embodiment, R₁ is halogen; in another embodiment, Rₛ₁ is —CN; in another embodiment, Rₛ₁ is —NO₂; in another embodiment, Rₛ₁ is —ORₐ; in another embodiment, Rₛ₁ is —SRₐ; in another embodiment, Rₛ₁ is —NR_{b}R_{c}; in another embodiment, Rₛ₁ is —C(O)Rₐ; in another embodiment, Rₛ₁ is —C(O)ORₐ; in another embodiment, Rₛ₁ is —C(O)NR_{b}R_{c}; in another embodiment, Rₛ₁ is C₁₋₆ alkyl; in another embodiment, Rₛ₁ is C₁₋₆ haloalkyl; in another embodiment, Rₛ₁ is C₂₋₆ alkenyl; in another embodiment, Rₛ₁ is C₂₋₆ alkynyl; in another embodiment, Rₛ₁ is C₃₋₇ cycloalkyl; in another embodiment, Rₛ₁ is 3- to 7-membered heterocyclyl; in another embodiment, Rₛ₁ is C₆₋₁₀ aryl; in another embodiment, Rₛ₁ is 5- to 10-membered heteroaryl.

Rₛ₂

In one embodiment, Rₛ₂ is H; in another embodiment, Rₛ₂ is halogen; in another embodiment, Rₛ₂ is —CN; in another embodiment, Rₛ₂ is —NO₂; in another embodiment, Rₛ₂ is —ORₐ; in another embodiment, Rₛ₂ is —SRₐ; in another embodiment, Rₛ₂ is —NR_{b}R_{c}; in another embodiment, Rₛ₂ is —C(O)Rₐ; in another embodiment, Rₛ₂ is —C(O)ORₐ; in another embodiment, Rₛ₂ is —C(O)NR_{b}R_{c}; in another embodiment, Rₛ₂ is C₁₋₆ alkyl; in another embodiment, Rₛ₂ is C₁₋₆ haloalkyl; in another embodiment, Rₛ₂ is C₂₋₆ alkenyl; in another embodiment, Rₛ₂ is C₂₋₆ alkynyl; in another embodiment, Rₛ₂ is C₃₋₇ cycloalkyl; in another embodiment, Rₛ₂ is 3- to 7-membered heterocyclyl; in another embodiment, Rₛ₂ is C₆₋₁₀ aryl; in another embodiment, Rₛ₂ is 5- to 10-membered heteroaryl.

Rₛ₃

In one embodiment, Rₛ₃ is H; in another embodiment, Rₛ₃ is halogen; in another embodiment, Rₛ₃ is —CN; in another embodiment, Rₛ₃ is —NO₂; in another embodiment, Rₛ₃ is —ORₐ; in another embodiment, Rₛ₃ is —SRₐ; in another embodiment, Rₛ₃ is —NR_{b}R_{c}; in another embodiment, Rₛ₃ is —C(O)Rₐ; in another embodiment, Rₛ₃ is —C(O)ORₐ; in another embodiment, Rₛ₃ is —C(O)NR_{b}R_{c}; in another embodiment, Rₛ₃ is C₁₋₆ alkyl; in another embodiment, Rₛ₃ is C₁₋₆ haloalkyl; in another embodiment, Rₛ₃ is C₂₋₆ alkenyl; in another embodiment, Rₛ₃ is C₂₋₆ alkynyl; in another embodiment, Rₛ₃ is C₃₋₇ cycloalkyl; in another embodiment, Rₛ₃ is 3- to 7-membered heterocyclyl; in another embodiment, Rₛ₃ is C₆₋₁₀ aryl; in another embodiment, Rₛ₃ is 5- to 10-membered heteroaryl.

Rₛ₄

In one embodiment, Rₛ₄ is H; in another embodiment, Rₛ₄ is halogen; in another embodiment, Rₛ₄ is —CN; in another embodiment, Rₛ₄ is —NO₂; in another embodiment, Rₛ₄ is —ORₐ; in another embodiment, Rₛ₄ is —SRₐ; in another embodiment, Rₛ₄ is —NR_{b}R_{c}; in another embodiment, Rₛ₄ is —C(O)Rₐ; in another embodiment, Rₛ₄ is —C(O)ORₐ; in another embodiment, Rₛ₄ is —C(O)NR_{b}R_{c}; in another embodiment, Rₛ₄ is C₁₋₆ alkyl; in another embodiment, Rₛ₄ is C₁₋₆ haloalkyl; in another embodiment, Rₛ₄ is C₂₋₆ alkenyl; in another embodiment, Rₛ₄ is C₂₋₆ alkynyl; in another embodiment, Rₛ₄ is C₃₋₇ cycloalkyl; in another embodiment, Rₛ₄ is 3- to 7-membered heterocyclyl; in another embodiment, Rₛ₄ is C₆₋₁₀ aryl; in another embodiment, Rₛ₄ is 5- to 10-membered heteroaryl.

m

In one embodiment, m=0; in another embodiment, m=1; in another embodiment, m=2; in another embodiment, m=3; in another embodiment, m=4; in another embodiment, m=5.

n

In one embodiment, n=0; in another embodiment, n=1; in another embodiment, n=2; in another embodiment, n=3; in another embodiment, n=4.

p

In one embodiment, p=0; in another embodiment, p=1; in another embodiment, p=2; in another embodiment, p=3; in another embodiment, p=4; in another embodiment, p=5; in another embodiment, p=6; in another embodiment, p=7; in another embodiment, p=8.

q

In one embodiment, q=0; in another embodiment, q=1; in another embodiment, q=2; in another embodiment, q=3; in another embodiment, q=4; in another embodiment, q=5.

Any technical solution in any one of the above embodiments, or any combination thereof, may be combined with any technical solution in any one of the above embodiments, or any combination thereof. For example, any technical solution of Ring A, or any combination thereof, may be combined with any technical solution of Ring B, L₁, L₂, Y, R₁, R₂, Rₛ₁-Rₛ₄, m, n, p, and q, etc or any combination thereof. The present disclosure is intended to include all combination of such technical solutions, which are not exhaustively listed here to save space.

In a more specific embodiment, the present disclosure provides the technical solution 1, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof:

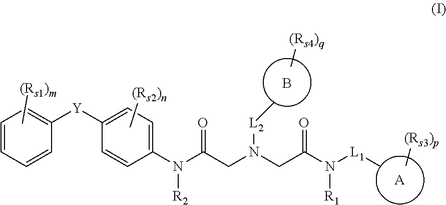

wherein:

Ring A is selected from C₃₋₇ cycloalkyl, 3- to 7-membered heterocyclyl, or C₆₋₁₀ aryl;

Ring B is selected from C₃₋₇ cycloalkyl, 3- to 7-membered heterocyclyl, C₆₋₁₀ aryl, or 5- to 10-membered heteroaryl;

L₁ is selected from a bond, —O—, —C(O)—, —CR′R″—, —CR′R″—CR′R″—, or —CR′R″—CR′R″—CR′R″—;

L₂ is selected from a bond, —C(O)—, —CR′R″—, —CR′R″—CR′R″—, or —CR′R″—CR′R″—CR′R″—;

Y is selected from O, S, NH, or CH₂;

R₁ is selected from H, —C(O)Rₐ, —C(O)ORₐ, —C(O)NR_{b}R_{c}, C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl;

R₂ is selected from H, C₁₋₆ alkyl, or C₁₋₆ haloalkyl;

Rₛ₁ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₂ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

Rₛ₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂ 6 alkynyl, C₃₋₇ cycloalkyl, 3- to 7-membered heterocyclyl, C₆₋₁₀ aryl, or 5- to 10-membered heteroaryl;

Rₛ₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

m=0, 1, 2, 3, 4, or 5;

n=0, 1, 2, 3, or 4;

p=0, 1, 2, 3, 4, 5, 6, 7, or 8;

q=0, 1, 2, 3, 4, or 5;

and wherein,

R′ and R″ are each independently selected from H, halogen, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, or C₂₋₆ alkynyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

wherein each of Y, R₁, R₂, Rₛ₁, Rₛ₂, Rₛ₃, and Rₛ₄ is optionally substituted by 1, 2 or 3 R groups, wherein R is independently selected from H, —OH, halogen, —NO₂, carbonyl, -L-CN, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ-NR_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, -L-O—C(S)—NR_{b}R_{c}, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl; wherein the said C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl is each further optionally substituted by one or more groups consisting of the following:

-L-CN, —NO₂, carbonyl, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—R_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, or -L-O—C(S)—NR_{b}R_{c};

L is selected from a chemical bond, —C₁₋₆ alkylene-, —C₂₋₆ alkenylene- or —C₂₋₆ alkynylene-;

x=0, 1 or 2.

In a more specific embodiment, the present disclosure provides the technical solution 2, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to technical solution 1, wherein R₂ is H.

In a more specific embodiment, the present disclosure provides the technical solution 3, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to technical solution 1 or 2, wherein R₁ is a group other than H.

In a more specific embodiment, the present disclosure provides the technical solution 4, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 3, wherein q=1, 2, 3, 4, or 5, and at least one of Rₛ₄ is selected from halogen, or C₁₋₆ haloalkyl.

In a more specific embodiment, the present disclosure provides the technical solution 5, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 4, wherein m=0, 1, 2, or 3, and R₁ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, or C₁₋₆ haloalkyl.

In a more specific embodiment, the present disclosure provides the technical solution 6, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 5, wherein p=0, 1, or 2, and Rₛ₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, or C₁₋₆ haloalkyl.

In a more specific embodiment, the present disclosure provides the technical solution 7, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 6, wherein Y is O.

In a more specific embodiment, the present disclosure provides the technical solution 8, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 7, wherein L₂ is —C(O)—.

In a more specific embodiment, the present disclosure provides the technical solution 9, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 8, wherein Ring A is selected from the following:

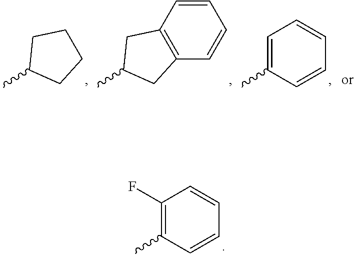

In a more specific embodiment, the present disclosure provides the technical solution 10, which refers to a compound of formula (I), or a pharmaceutically acceptable salt, an enantiomer, a diastereomer, a 25 racemate, a solvate, a hydrate, a polymorph, a prodrug, or an isotope variant thereof, and mixtures thereof according to any one of technical solutions 1 to 9, wherein Ring B is selected from the following:

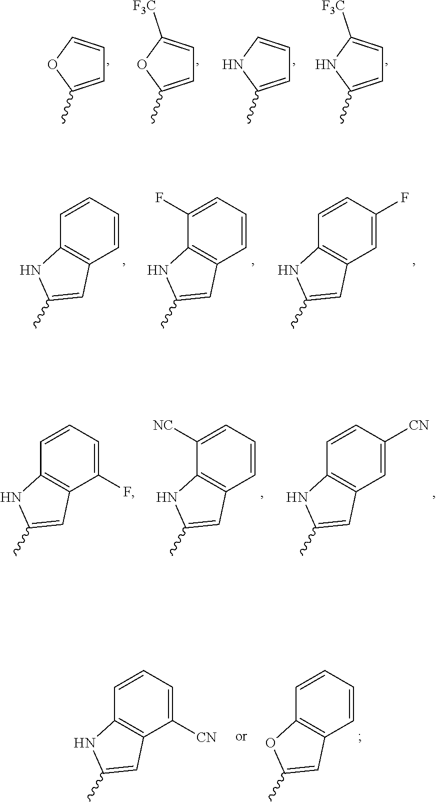

preferably, wherein Ring B is selected from the following:

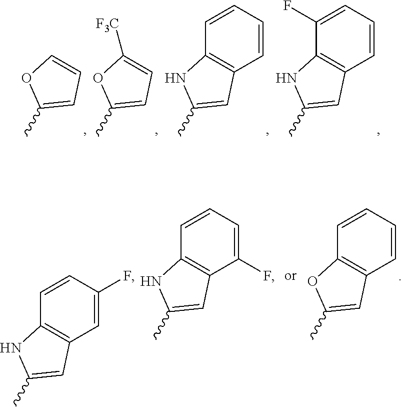

In a more specific embodiment, the present disclosure provides the technical solution 11, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formulae (II-1) to (II-4):

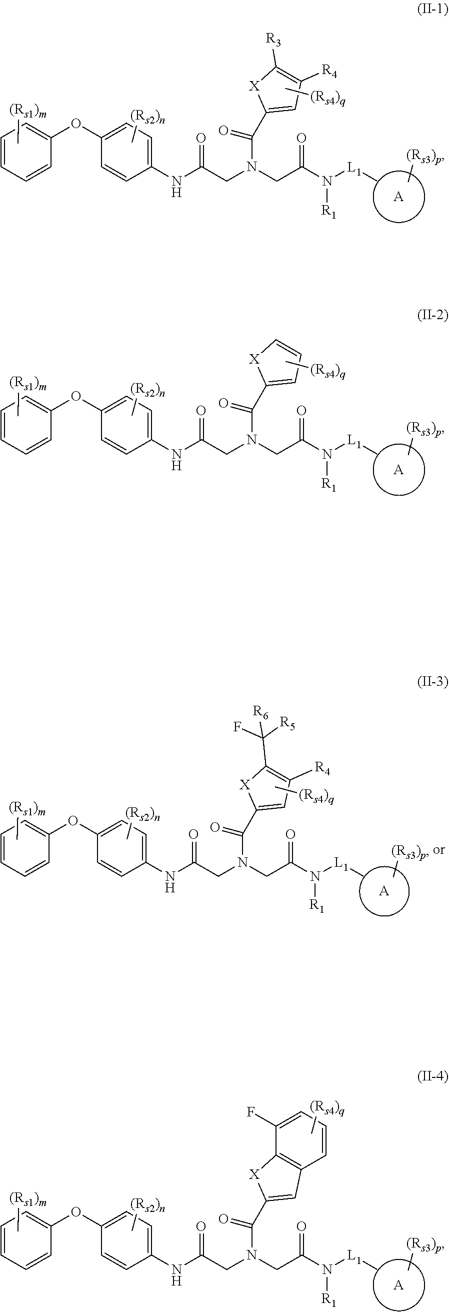

wherein:

X is selected from O, S, NH or CH₂;

R₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃a cycloalkyl, 3- to 7-membered heterocyclyl, C₆₋₁₀ aryl, or 5- to 10-membered heteroaryl;

R₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

or, R₃ and R₄ are linked to form a C₃₋₇ cycloalkyl, 3- to 7-membered heterocyclyl, C₆₋₁₀ aryl, or 5- to 10-membered heteroaryl;

R₅ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;

R₆ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;

or, R₅ and R₄ are linked to form a C₃₋₇ cycloalkyl, 3- to 7-membered heterocyclyl, C₆₋₁₀ aryl, or 5- to 10-membered heteroaryl; and

other variables are as defined in any one of technical solutions 1 to 10.

In a more specific embodiment, the present disclosure provides the technical solution 12, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formulae (III-1) to (III-3):

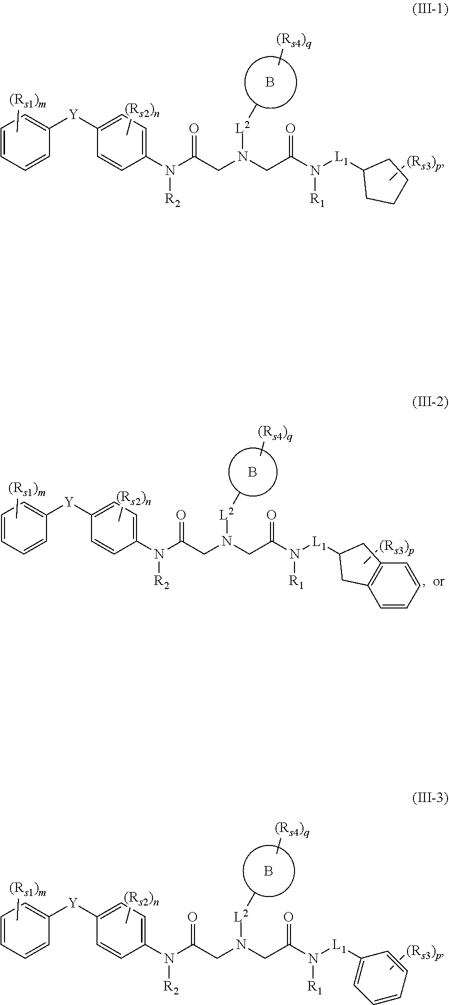

wherein the variables are as defined in any one of technical solutions 1 to 10.

In a more specific embodiment, the present disclosure provides the technical solution 13, which refers to a compound of formula (I), or a pharmaceutically acceptable salt, an enantiomer, a diastereomer, a 15 racemate, a solvate, a hydrate, a polymorph, a prodrug, or an isotope variant thereof, and mixtures thereof according to any one of technical solutions 1 to 10, which is the compound of formulae (IV-1) to (IV-3):

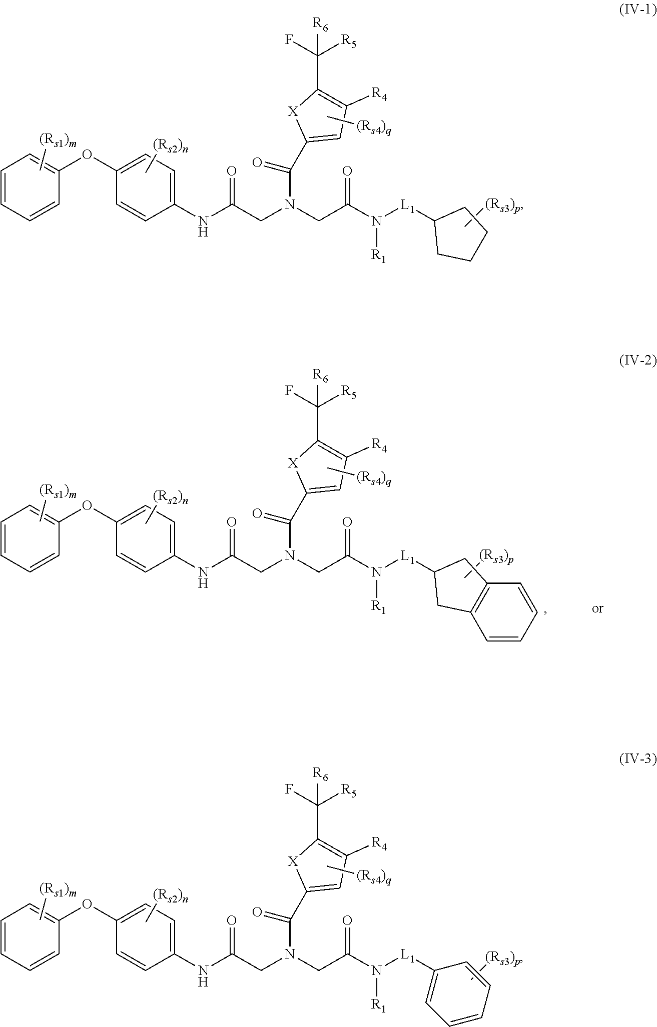

wherein:

X is selected from O, S, or NH;

R₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, or C₂₋₆ alkynyl;

R₅ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;

R₆ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;

or, R₅ and R₄ are linked to form a C₆₋₁₀ aryl, or 5- to 10-membered heteroaryl; and

other variables are as defined in any one of technical solutions 1 to 10.

In a more specific embodiment, the present disclosure provides the technical solution 14, which refers to compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formula (III-1):

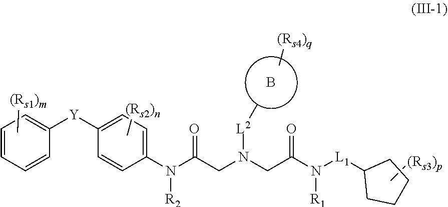

wherein,

Ring B is 5- to 10-membered heteroaryl;

L₁ is a bond;

L₂ is selected from a bond, —C(O)—, or —CR′R″—;

Y is selected from O, S, or NH;

R₁ is selected from H, C₁₋₆ alkyl, or C₁₋₆ haloalkyl;

R₂ is selected from H, C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

R′ and R″ are each independently selected from H, halogen, C₁₋₆ alkyl, or C₁₋₆ haloalkyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

Ring B is selected from the following:

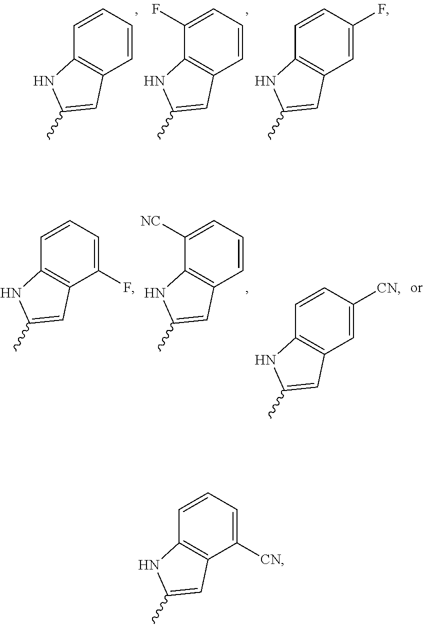

L₁ is a bond;

L₂ is —C(O)—;

Y is O;

R₁ is selected from H, C₁₋₆ alkyl, or C₁₋₆ haloalkyl;

R₂ is H;

Rₛ₁ is selected from H, halogen, C₁₋₆ alkyl, or C₁₋₆ haloalkyl;

Rₛ₂ is H;

Rₛ₃ is H;

Rₛ₄ is H;

m=0, 1, or 2;

n=0, 1, or 2;

p=0, 1, or 2;

q=0, 1, or 2.

In a more specific embodiment, the present disclosure provides the technical solution 15, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formula (III-2):

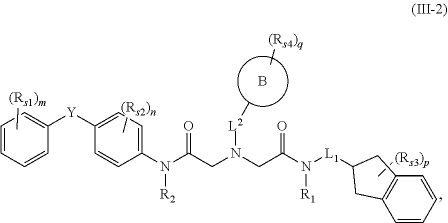

wherein,

Ring B is 5- to 10-membered heteroaryl;

L₁ is a bond;

L₂ is selected from a bond, —C(O)—, or —CR′R″—;

Y is selected from O, S, or NH;

R₁ is selected from H, C₁₋₆ alkyl, or C₁₋₆ haloalkyl;

R₂ is selected from H, C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

R′ and R″ are each independently selected from H, halogen, C₁₋₆ alkyl, or C₁₋₆ haloalkyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

Ring B is selected from the following:

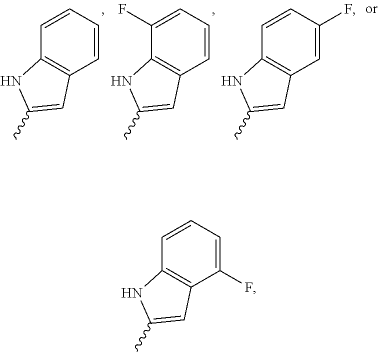

L₁ is a bond;

L₂ is —C(O)—;

Y is O;

R₁ is selected from H, C₁₋₆ alkyl, or C₁₋₆ haloalkyl;

R₂ is selected from H, C₁₋₆ alkyl, or C₁₋₆ haloalkyl;

Rₛ₁ is selected from H, or halogen;

Rₛ₂ is H;

Rₛ₃ is H;

Rₛ₄ is H;

m=0, 1, or 2;

n=0, 1, or 2;

p=0, 1, or 2;

q=0, 1, or 2;

alternatively,

Ring B is 5- to 6-membered heteroaryl;

L₁ is a bond;

L₂ is selected from a bond, —C(O)—, or —CR′R″—;

Y is selected from O, S, or NH;

R₁ is C₁₋₆ alkyl, or C₁₋₆ haloalkyl;

R₂ is H;

R₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

Ring B is selected from the following:

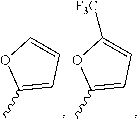

L₁ is a bond;

L₂ is —C(O)—;

Y is O;

R₁ is C₁₋₆ alkyl, or C₁₋₆ haloalkyl;

R₂ is H;

Rₛ₁ is selected from H, or halogen;

Rₛ₂ is H;

Rₛ₃ is H;

Rₛ₄ is H;

m=0, 1, or 2;

n=0, 1, or 2;

p=0, 1, or 2;

q=0, 1, or 2.

In a more specific embodiment, the present disclosure provides the technical solution 16, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formula (III-3):

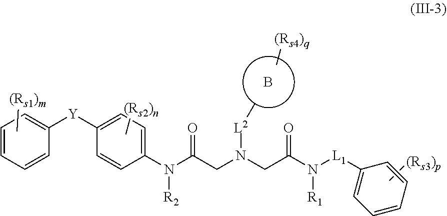

wherein:

Ring B is 5- to 10-membered heteroaryl;

L₁ is —CR′R″—, —CR′R″—CR′R″—, or —CR′R″—CR′R″—CR′R″—;

L₂ is selected from a bond, —C(O)—, or —CR′R″—;

Y is selected from O, S, or NH;

R₁ is selected from H, C₁₋₆ alkyl, or C₁₋₆ haloalkyl;

R₂ is selected from H, C₁₋₆ alkyl, or C₁₋₆ haloalkyl;

R₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

R′ and R″ are each independently selected from H, halogen, C₁₋₆ alkyl, or C₁₋₆ haloalkyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, Rₑ and N atom are taken together to form 3- to 7-membered heterocyclyl;

alternatively,

Ring B is selected from the following:

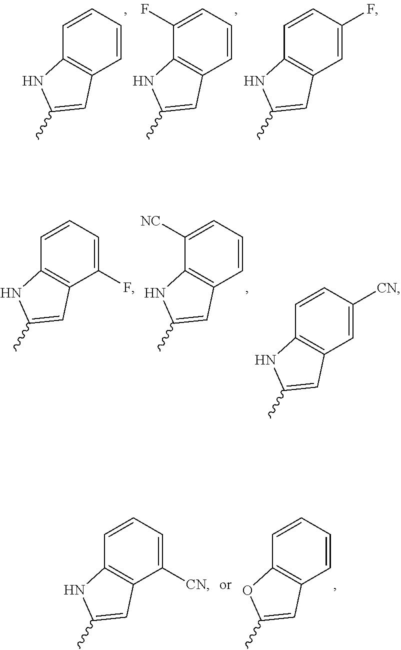

L₁ is —CR′R″—;

L₂ is —C(O)—;

Y is O;

R₁ is selected from H, C₁₋₆ alkyl, or C₁₋₆ haloalkyl;

R₂ is selected from H, C₁₋₆ alkyl, or C₁₋₆ haloalkyl;

Rₛ₁ is selected from H, halogen, C₁₋₆ alkyl, or C₁₋₆ haloalkyl;

Rₛ₂ is H;

Rₛ₃ is H, or halogen;

Rₛ₄ is H;

m=0, 1, or 2;

n=0, 1, or 2;

p=0, 1, or 2;

q=0, 1, or 2;

R′ and R″ are each independently selected from H, or halogen;

alternatively,

Ring B is 5- to 10-membered heteroaryl;

L₁ is a bond;

L₂ is selected from a bond, —C(O)—, or —CR′R″—;

Y is selected from O, S, or NH;

R₁ is H;

R₂ is H;

R₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

Ring B is selected from the following:

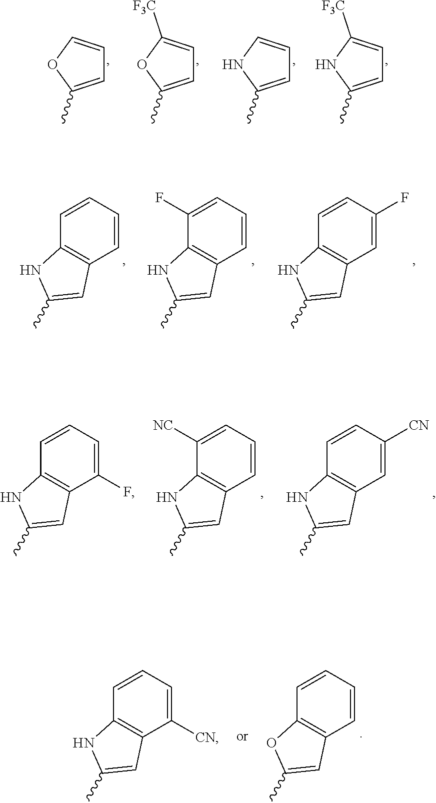

L₁ is a bond;

L₂ is —C(O)—;

Y is O;

R₁ is H;

R₂ is H;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is H;

Rₛ₃ is H;

Rₛ₄ is H;

m=0, 1, or 2;

n=0, 1, or 2;

p=0, 1, or 2;

q=0, 1, or 2;

Rₐ is independently selected from H, C₁₋₆ alkyl, or C₁₋₆ haloalkyl;

R_{b} and R_{c} are each independently selected from H, C₁₋₆ alkyl, or C₁₋₆ haloalkyl; alternatively,

Ring B is 5- to 6-membered heteroaryl;

L₁ is a —CR′R″—, —CR′R″—CR′R″—, or —CR′R″—CR′R″—CR′R″—;

L₂ is selected from a bond, —C(O)—, or —CR′R″—;

Y is selected from O, S, or NH;

R₁ is C₁₋₆ alkyl, or C₁₋₆ haloalkyl;

R₂ is H;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

R′ and R″ are each independently selected from H, halogen, C₁₋₆ alkyl, or C₁₋₆ haloalkyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

Ring B is selected from the following:

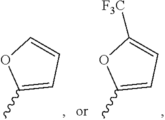

L₁ is a —CR′R″—;

L₂ is —C(O)—;

Y is O;

R₁ is C₁₋₆ alkyl, or C₁₋₆ haloalkyl;

R₂ is H;

Rₐ1 is selected from H, or halogen;

Rₛ₂ is H;

Rₛ₃ is H, or halogen;

Rₛ₄ is H;

m=0, 1, or 2;

n=0, 1, or 2;

p=0, 1, or 2;

q=0, 1, or 2;

R′ and R″ are each independently selected from H, or halogen.

In a more specific embodiment, the present disclosure provides the technical solution 17, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formula (IV-1):

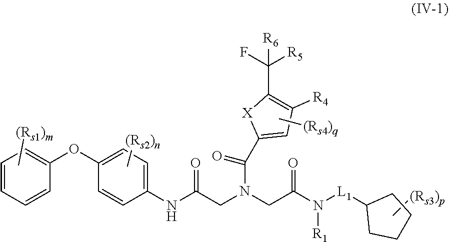

wherein,

X is selected from O, S, or NH;

R₄, R₅ and R₆ are linked together with the atoms they are attached to form a C₆₋₁₀ aryl, or 5- to 10-membered heteroaryl;

L₁ is a bond;

R₁ is C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

X is NH;

R₄, R₅ and R₆ are linked together with the atoms they are attached to form a phenyl;

L₁ is a bond;

R₁ is C₁₋₆ alkyl, or C₁₋₆ haloalkyl;

Rₛ₁ is selected from H, or halogen;

Rₛ₂ is H;

Rₛ₃ is H;

Rₛ₄ is H;

m=0, 1, or 2;

n=0, 1, or 2;

p=0, 1, or 2;

q=0, 1, or 2;

alternatively,

X is selected from O, S, or NH;

L₁ is a bond;

R₁ is H;

R₄ is selected from H, halogen, —CN, —NO₂, —ORₐ, —SRₐ, —NR_{b}R_{c}, C₁₋₆ alkyl, or C₁₋₆ haloalkyl;

R₅ is selected from H, halogen, —CN, —NO₂, —ORₐ, —SRₐ, —NR_{b}R_{c}, C₁₋₆ alkyl, or C₁₋₆ haloalkyl;

R₆ is selected from H, halogen, —CN, —NO₂, —ORₐ, —SRₐ, —NR_{b}R_{c}, C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

X is NH;

L₁ is a bond;

R₁ is H;

R₄ is selected from H, or halogen;

R₅ is selected from H, or halogen;

R₆ is selected from H, or halogen;

Rₛ₁ is selected from H, halogen, C₁₋₆ alkyl, or C₁₋₆ haloalkyl;

Rₛ₂ is H;

Rₛ₃ is H;

Rₛ₄ is H;

m=0, 1, or 2;

n=0, 1, or 2;

p=0, 1, or 2;

q=0, 1, or 2.

In a more specific embodiment, the present disclosure provides the technical solution 18, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formula (IV-2):

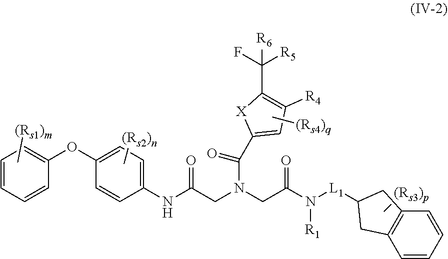

X is selected from O, S, or NH;

R₄, R₅ and R₆ are linked together with the atoms they are attached to form a C₆₋₁₀ aryl, or 5- to 10-membered heteroaryl;

L₁ is a bond;

R₁ is selected from H, C₁₋₆ alkyl, or C₁₋₆ haloalkyl; alternatively, R₁ is H;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

X is NH;

R₄, R₅ and R₆ are linked together with the atoms they are attached to form a phenyl;

L₁ is a bond;

R₁ is selected from H, C₁₋₆ alkyl, or C₁₋₆ haloalkyl; alternatively, R₁ is H;

R₁ is selected from H, or halogen;

Rₛ₂ is H;

Rₛ₃ is H;

Rₛ₄ is H;

m=0, 1, or 2;

n=0, 1, or 2;

p=0, 1, or 2;

q=0, 1, or 2;

alternatively,

X is selected from O, S, or NH;

L₁ is a bond;

R₁ is C₁₋₆ alkyl, or C₁₋₆ haloalkyl;

R₄ is selected from H, halogen, —CN, —NO₂, —ORₐ, —SRₐ, —NR_{b}R_{c}, C₁₋₆ alkyl, or C₁₋₆ haloalkyl;

R₅ is selected from H, halogen, —CN, —NO₂, —ORₐ, —SRₐ, —NR_{b}R_{c}, C₁₋₆ alkyl, or C₁₋₆ haloalkyl;

R₆ is selected from H, halogen, —CN, —NO₂, —ORₐ, —SRₐ, —NR_{b}R_{c}, C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl; Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

X is O;

L₁ is a bond;

R₁ is C₁₋₆ alkyl, or C₁₋₆ haloalkyl;

R₄ is selected from H, or halogen;

R₅ is selected from H, or halogen;

R₆ is selected from H, or halogen;

Rₛ₁ is selected from H, or halogen;

Rₛ₂ is H;

Rₛ₃ is H;

Rₛ₄ is H;

m=0, 1, or 2;

n=0, 1, or 2;

p=0, 1, or 2;

q=0, 1, or 2.

In a more specific embodiment, the present disclosure provides the technical solution 19, which refers to a compound of formula (I), or a pharmaceutically acceptable salt, an enantiomer, a diastereomer, a racemate, a solvate, a hydrate, a polymorph, a prodrug, or an isotope variant thereof, and mixtures thereof according to any one of technical solutions 1 to 10, which is the compound of formula (IV-3):

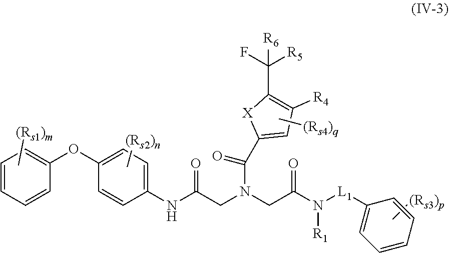

wherein,

X is selected from O, S, or NH;

R₄, R₅ and R₆ are linked together with the atoms they are attached to form a C₆₋₁₀ aryl, or 5- to 10-membered heteroaryl;

L₁ is a —CR′R″—, —CR′R″—CR′R″—, or —CR′R″—CR′R″—CR′R″—;

R₁ is H, C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

R′ and R″ are each independently selected from H, halogen, C₁₋₆ alkyl, or C₁₋₆ haloalkyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

X is NH;

R₄, R₅ and R₆ are linked together with the atoms they are attached to form a phenyl;

L₁ is —CR′R″—;

R₁ is H, C₁₋₆ alkyl, or C₁₋₆ haloalkyl;

Rₛ₁ is selected from H, halogen, C₁₋₆ alkyl, or C₁₋₆ haloalkyl;

Rₛ₂ is H;

Rₛ₃ is H, or halogen;

Rₛ₄ is H;

m=0, 1, or 2;

n=0, 1, or 2;

p=0, 1, or 2;

q=0, 1, or 2;

R′ and R″ are each independently selected from H, or halogen;

alternatively,

X is selected from O, S, or NH;

L₁ is a bond;

R₁ is H;

R₄ is selected from H, halogen, —CN, —NO₂, —ORₐ, —SRₐ, —NR_{b}R_{c}, C₁₋₆ alkyl, or C₁₋₆ haloalkyl;

R₅ is selected from H, halogen, —CN, —NO₂, —ORₐ, —SRₐ, —NR_{b}R_{c}, C₁₋₆ alkyl, or C₁₋₆ haloalkyl;

R₆ is selected from H, halogen, —CN, —NO₂, —ORₐ, —SRₐ, —NR_{b}R_{c}, C₁₋₆ alkyl, or C₁₋₆ haloalkyl; or R₄, R₅ and R₆ are linked together with the atoms they are attached to form a C₆₋₁₀ aryl, or 5- to 10-membered heteroaryl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl; or, R_{b}, R_{c} and N atom are taken together to form 3- to 7-membered heterocyclyl;

alternatively,

X is NH;

L₁ is a bond;

R₁ is H;

R₄ is selected from H, or halogen;

R₅ is selected from H, or halogen;

R₆ is selected from H, or halogen;

or R₄, R₅ and R₆ are linked together with the atoms they are attached to form a phenyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is H;

Rₛ₃ is H;

Rₛ₄ is H;

m=0, 1, or 2;

n=0, 1, or 2;

p=0, 1, or 2;

q=0, 1, or 2;

Rₐ is independently selected from H, C₁₋₆ alkyl, or C₁₋₆ haloalkyl;

R_{b} and R_{c} are each independently selected from H, C₁₋₆ alkyl, or C₁₋₆ haloalkyl;

alternatively,

X is selected from O, S, or NH;

L₁ is a —CR′R″—, —CR′R″—CR′R″—, or —CR′R″—CR′R″—CR′R″—;

R₁ is C₁₋₆ alkyl, or C₁₋₆ haloalkyl;

R₄ is selected from H, halogen, —CN, —NO₂, —ORₐ, —SRₐ, —NR_{b}R_{c}, C₁₋₆ alkyl, or C₁₋₆ haloalkyl;

R₅ is selected from H, halogen, —CN, —NO₂, —ORₐ, —SRₐ, —NR_{b}R_{c}, C₁₋₆ alkyl, or C₁₋₆ haloalkyl;

R₆ is selected from H, halogen, —CN, —NO₂, —ORₐ, —SRₐ, —NR_{b}R_{c}, C₁₋₆ alkyl, or C₁₋₆ haloalkyl;

Rₛ₁ is selected from H, halogen, —CN, —NO₂, —ORₐ, —SRₐ, —NR_{b}R_{c}, C₁₋₆ alkyl, or C₁₋₆ haloalkyl;

Rₛ₂ is selected from H, halogen, —CN, —NO₂, —ORₐ, —SRₐ, —NR_{b}R_{c}, C₁₋₆ alkyl, or C₁₋₆ haloalkyl;

Rₛ₃ is selected from H, halogen, —CN, —NO₂, —ORₐ, —SRₐ, —NR_{b}R_{c}, C₁₋₆ alkyl, or C₁₋₆ haloalkyl;

Rₛ₄ is selected from H, halogen, —CN, —NO₂, —ORₐ, —SRₐ, —NR_{b}R_{c}, C₁₋₆ alkyl, or C₁₋₆ haloalkyl;

m=0, 1, 2, or 3;

n=0, 1, 2, or 3;

p=0, 1, 2, or 3;

q=0, 1, 2, or 3;

R′ and R″ are each independently selected from H, halogen, C₁₋₆ alkyl, or C₁₋₆ haloalkyl;

Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;

R_{b} and R_{c} are each independently selected from H, C₁₋₆ alkyl, C₁₋₆ haloalkyl, C₂₋₆ alkenyl, C₂_s alkynyl, C₃₋₇ cycloalkyl, 3- to 7-membered heterocyclyl, C₆-1a aryl, or 5- to 10-membered heteroaryl; or, R_{b}, R_{c} and N atom are taken together to form 3- to 7-membered heterocyclyl;

alternatively,

X is O;

L₁ is —CR′R″—;

R₁ is C₁₋₆ alkyl, or C₁₋₆ haloalkyl;

R₄ is selected from H, or halogen;

R₅ is selected from H, or halogen;

R₆ is selected from H, or halogen;

Rₛ₁ is selected from H, or halogen;

Rₛ₂ is H;

Rₛ₃ is H, or halogen;

Rₛ₄ is H;

m=0, 1, or 2;

n=0, 1, or 2;

p=0, 1, or 2;

q=0, 1, or 2;

R′ and R″ are each independently selected from H, or halogen.

The compounds of the present disclosure may include one or more asymmetric centers, and thus may exist in a variety of stereoisomeric forms, for example, enantiomers and/or diastereomers. For example, the compounds of the present disclosure may be in the form of an individual enantiomer, diastereomer or geometric isomer (e.g., cis- and trans-isomers), or may be in the form of a mixture of stereoisomers, including racemic mixture and a mixture enriched in one or more stereoisomers. The isomers can be separated from the mixture by the methods known to those skilled in the art, including chiral high pressure liquid chromatography (HPLC) and the formation and crystallization of chiral salts; or preferred isomers can be prepared by asymmetric synthesis.

It will be understood by those skilled in the art that the organic compounds can form complexes with solvents in which they are reacted or from which they are precipitated or crystallized. These complexes are known as “solvates.” Where the solvent is water, the complex is known as “hydrate.” The present disclosure encompasses all solvates of the compounds of the present disclosure.

The term “solvate” refers to forms of a compound or a salt thereof, which are associated with a solvent, usually by a solvolysis reaction. This physical association may include hydrogen bonding. Conventional solvents include water, methanol, ethanol, acetic acid, DMSO, THF, diethyl ether, etc. The compounds described herein can be prepared, for example, in crystalline form, and can be solvated. Suitable solvates include pharmaceutically acceptable solvates and further include both stoichiometric solvates and non-stoichiometric solvates. In some cases, the solvates will be capable of isolation, for example, when one or more solvent molecules are incorporated into the crystal lattice of a crystalline solid. “Solvate” includes both solution-phase and isolatable solvates. Representative solvates include hydrates, ethanolates and methanolates.

The term “hydrate” refers to a compound that is associated with water. Generally, the number of water molecules contained in a hydrate of a compound is in a definite ratio to the number of the compound molecules in the hydrate. Therefore, hydrates of a compound can be represented, for example, by a general formula R.x H₂O, wherein R is the compound, and x is a number greater than 0. Given compounds can form more than one type of hydrates, including, for example, monohydrates (x is 1), lower hydrates (x is a number greater than 0 and smaller than 1, for example, hemihydrates (R.0.5 H₂O)) and polyhydrates (x is a number greater than 1, for example, dihydrates (R.2H₂O) and hexahydrates (R.6H₂O)).

Compounds of the present disclosure may be in an amorphous or a crystalline form (polymorph). Furthermore, the compounds of the present disclosure may exist in one or more crystalline forms. Therefore, the present disclosure includes all amorphous or crystalline forms of the compounds of the present disclosure within its scope. The term “polymorph” refers to a crystalline form of a compound (or a salt, hydrate or solvate thereof) in a particular crystal packing arrangement. All polymorphs have the same elemental composition. Different crystalline forms generally have different X-ray diffraction patterns, infrared spectra, melting points, density, hardness, crystal shapes, optical and electrical properties, stability, and solubility. Recrystallization solvents, rate of crystallization, storage temperatures, and other factors may cause one crystalline form to dominate. Various polymorphs of a compound can be prepared by crystallization under different conditions.

The present disclosure also comprises compounds that are labeled with isotopes, which are equivalent to those described in formula (I), but one or more atoms are replaced by atoms having an atom mass or mass number that are different from that of atoms that are common in nature. Examples of isotopes which may be introduced into the compounds of the disclosure include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl, respectively. Compounds of the present disclosure that comprise the above isotopes and/or other isotopes of other atoms, prodrugs thereof and pharmaceutically acceptable salts of said compounds or prodrugs all are within the scope of the present disclosure. Certain isotope-labeled compounds of the present disclosure, such as those incorporating radioactive isotopes (e.g., ³H and ¹⁴C), can be used for the measurement of the distribution of drug and/or substrate in tissue. Tritium, which is ³H and carbon-14, which is ¹⁴C isotope, are particularly preferred, because they are easy to prepare and detect. Furthermore, replaced by heavier isotopes, such as deuterium, which is ²H, may provide therapeutic benefits due to the higher metabolic stability, such as prolonging the half-life in vivo or decreasing the dosage requirements, and thus may be preferred in some cases. Isotope-labeled compounds of formula (I) of the present disclosure and prodrugs thereof can be prepared generally by using readily available isotope-labeled reagents to replace non-isotope-labeled reagents in the following schemes and/or the procedures disclosed in the examples and preparation examples.

In addition, prodrugs are also included within the context of the present disclosure. The term “prodrug” as used herein refers to a compound that is converted into an active form that has medical effects in vivo by, for example, hydrolysis in blood. Pharmaceutically acceptable prodrugs are described in T. Higuchi and V. Stella, Prodrugs as Novel Delivery Systems, A.C.S. Symposium Series, Vol. 14, Edward B. Roche, ed., Bioreversible Carriers in Drug Design, American Pharmaceutical Association and Pergamon Press, 1987, and D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

The prodrugs are any covalently bonded compounds of the present disclosure, which release the parent compound in vivo when the prodrug is administered to a patient. Prodrugs are typically prepared by modifying functional groups in such a way that the modifications can be cleaved either by routine manipulation or decompose in vivo to yield the parent compound. Prodrugs include, for example, compounds of the present disclosure wherein the hydroxyl, amino or sulfhydryl groups are bonded to any group that, when administered to a patient, cleaves to form the hydroxyl, amino or sulfhydryl groups. Thus, representative examples of prodrugs include (but are not limited to) the acetate/acetamide, formate/formamide and benzoate/benzamide derivatives of the hydroxyl, amino or sulfhydryl functional groups of the compounds of formula (I). Furthermore, in the case of carboxylic acid (—COOH), esters such as methyl esters and ethyl esters, etc. can be employed. The ester itself may be active in their own and/or hydrolyzable under in vivo conditions in the human body. Suitable pharmaceutically acceptable in vivo hydrolysable ester groups include those groups that can readily break down in the human body to release the parent acids or salts thereof.

The present disclosure also provides a pharmaceutical formulation comprising a therapeutically effective amount of a compound of formula (I), or therapeutically acceptable salts thereof, and pharmaceutically acceptable carriers, diluents or excipients thereof. All of these forms belong to the present disclosure.

The preferred compounds disclosed herein include but are not limited to the following compounds, or a pharmaceutically acceptable salt, an enantiomer, a diastereomer, a racemate, a solvate, a hydrate, a polymorph, a prodrug, or an isotope variant thereof, and mixtures thereof:

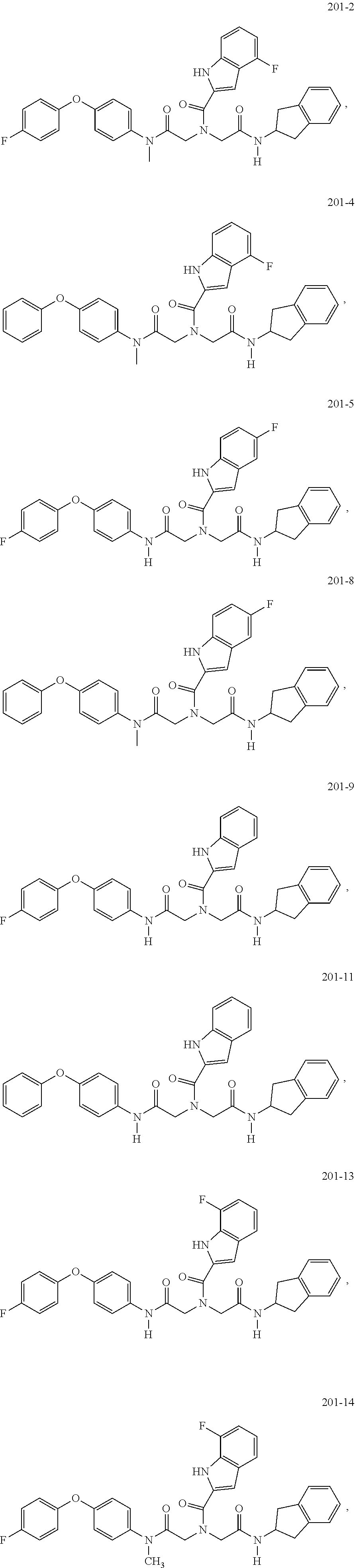

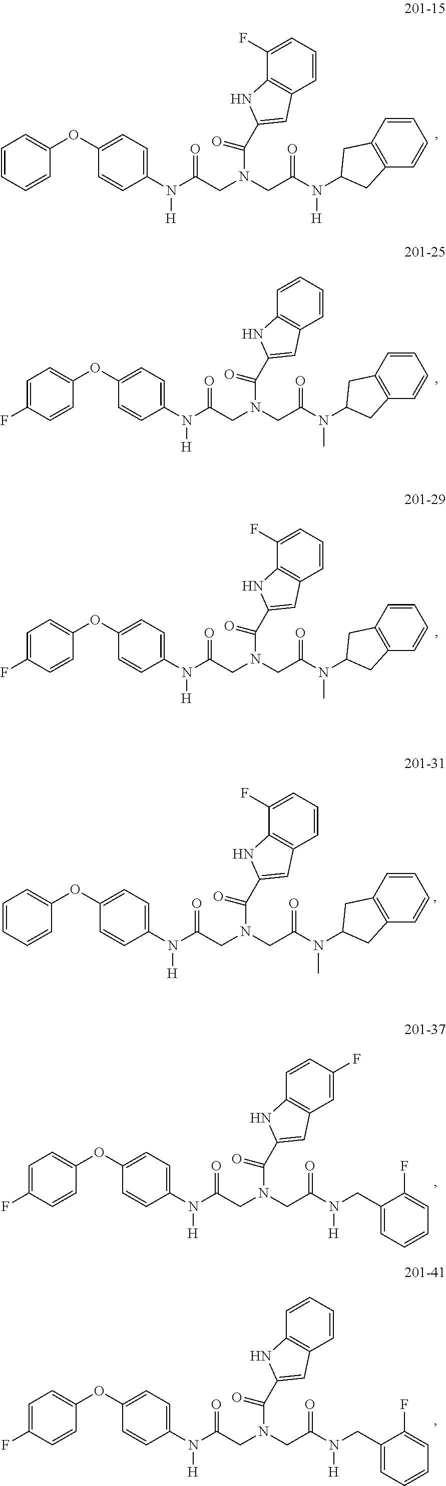 [SMILES: O=C(CN(CC(=O)NC1Cc2ccccc2C1)C(=O)c1cc2cccc(F)c2[nH]1)Nc1ccc(Oc2ccccc2)cc1 ; CN(C(=O)CN(CC(=O)Nc1ccc(Oc2ccc(F)cc2)cc1)C(=O)c1cc2ccccc2[nH]1)C1Cc2ccccc2C1 ; CN(C(=O)CN(CC(=O)Nc1ccc(Oc2ccc(F)cc2)cc1)C(=O)c1cc2cccc(F)c2[nH]1)C1Cc2ccccc2C1 ; CN(C(=O)CN(CC(=O)Nc1ccc(Oc2ccccc2)cc1)C(=O)c1cc2cccc(F)c2[nH]1)C1Cc2ccccc2C1 ; O=C(CN(CC(=O)Nc1ccc(Oc2ccc(F)cc2)cc1)C(=O)c1cc2cc(F)ccc2[nH]1)NCc1ccccc1F ; O=C(CN(CC(=O)Nc1ccc(Oc2ccc(F)cc2)cc1)C(=O)c1cc2ccccc2[nH]1)NCc1ccccc1F]

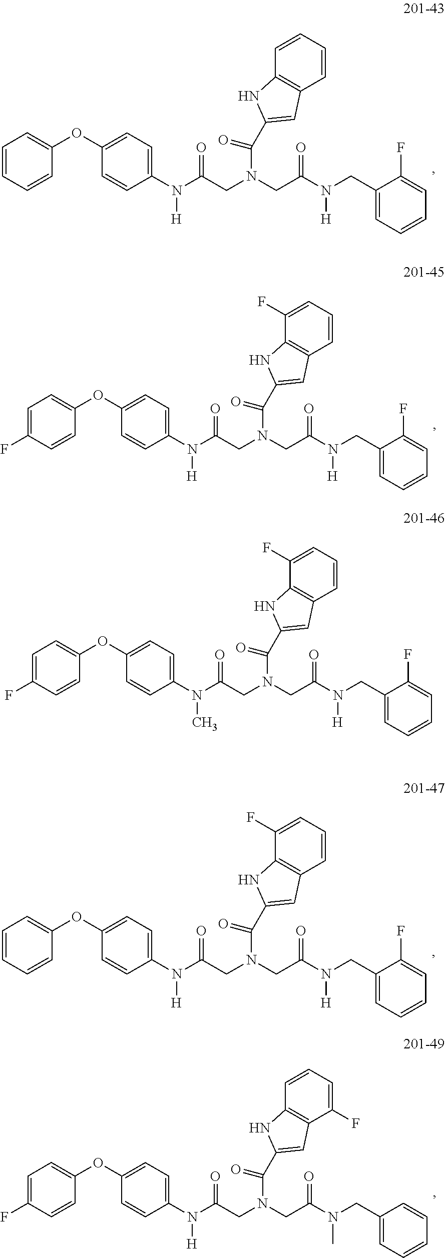 [SMILES: O=C(CN(CC(=O)Nc1ccc(Oc2ccccc2)cc1)C(=O)c1cc2ccccc2[nH]1)NCc1ccccc1F ; O=C(CN(CC(=O)Nc1ccc(Oc2ccc(F)cc2)cc1)C(=O)c1cc2cccc(F)c2[nH]1)NCc1ccccc1F ; CN(C(=O)CN(CC(=O)NCc1ccccc1F)C(=O)c1cc2cccc(F)c2[nH]1)c1ccc(Oc2ccc(F)cc2)cc1 ; O=C(CN(CC(=O)Nc1ccc(Oc2ccccc2)cc1)C(=O)c1cc2cccc(F)c2[nH]1)NCc1ccccc1F ; CN(Cc1ccccc1)C(=O)CN(CC(=O)Nc1ccc(Oc2ccc(F)cc2)cc1)C(=O)c1cc2c(F)cccc2[nH]1]

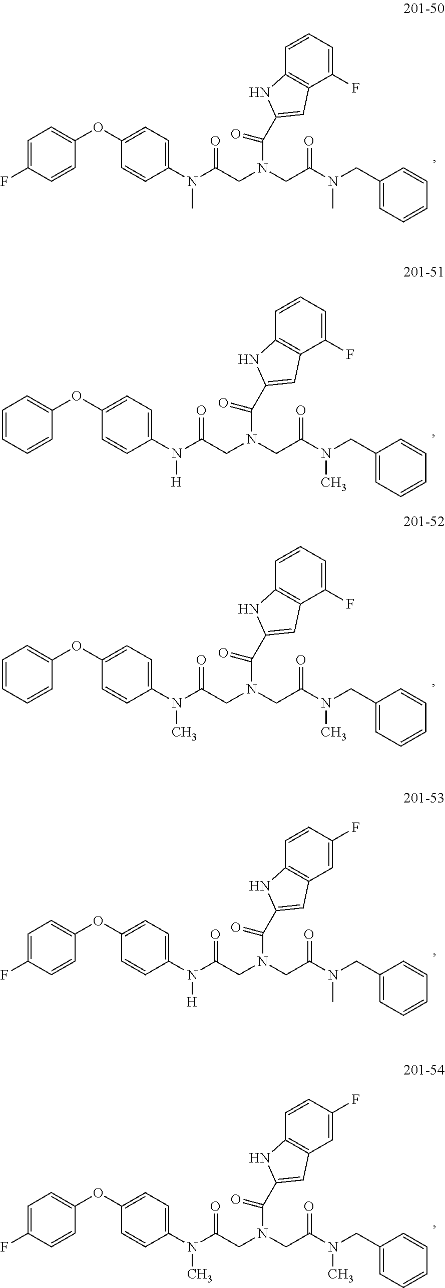 [SMILES: CN(Cc1ccccc1)C(=O)CN(CC(=O)N(C)c1ccc(Oc2ccc(F)cc2)cc1)C(=O)c1cc2c(F)cccc2[nH]1 ; CN(Cc1ccccc1)C(=O)CN(CC(=O)Nc1ccc(Oc2ccccc2)cc1)C(=O)c1cc2c(F)cccc2[nH]1 ; CN(Cc1ccccc1)C(=O)CN(CC(=O)N(C)c1ccc(Oc2ccccc2)cc1)C(=O)c1cc2c(F)cccc2[nH]1 ; CN(Cc1ccccc1)C(=O)CN(CC(=O)Nc1ccc(Oc2ccc(F)cc2)cc1)C(=O)c1cc2cc(F)ccc2[nH]1 ; CN(Cc1ccccc1)C(=O)CN(CC(=O)N(C)c1ccc(Oc2ccc(F)cc2)cc1)C(=O)c1cc2cc(F)ccc2[nH]1]

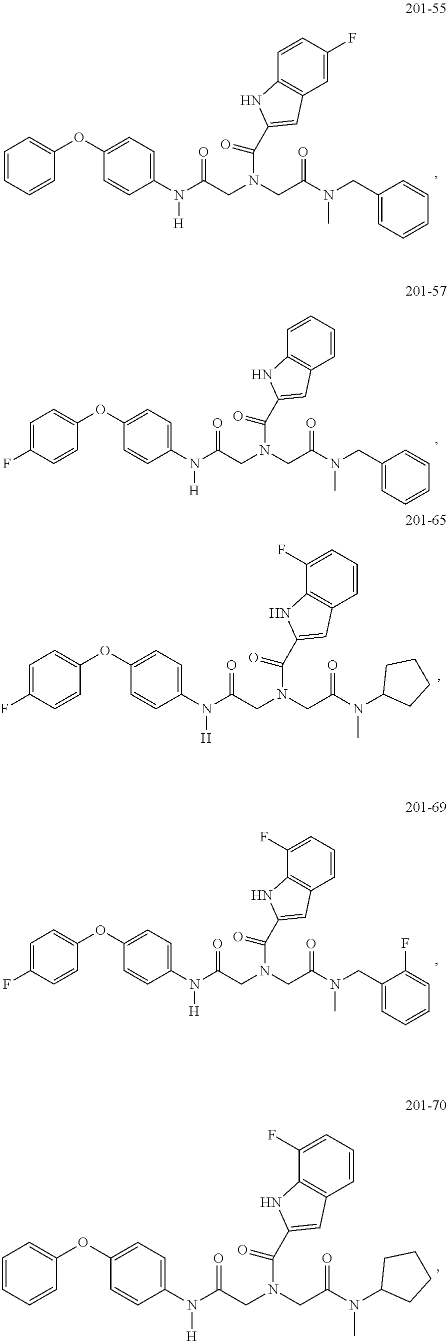 [SMILES: CN(Cc1ccccc1)C(=O)CN(CC(=O)Nc1ccc(Oc2ccccc2)cc1)C(=O)c1cc2cc(F)ccc2[nH]1 ; CN(Cc1ccccc1)C(=O)CN(CC(=O)Nc1ccc(Oc2ccc(F)cc2)cc1)C(=O)c1cc2ccccc2[nH]1 ; CN(C(=O)CN(CC(=O)Nc1ccc(Oc2ccc(F)cc2)cc1)C(=O)c1cc2cccc(F)c2[nH]1)C1CCCC1 ; CN(Cc1ccccc1F)C(=O)CN(CC(=O)Nc1ccc(Oc2ccc(F)cc2)cc1)C(=O)c1cc2cccc(F)c2[nH]1 ; CN(C(=O)CN(CC(=O)Nc1ccc(Oc2ccccc2)cc1)C(=O)c1cc2cccc(F)c2[nH]1)C1CCCC1]

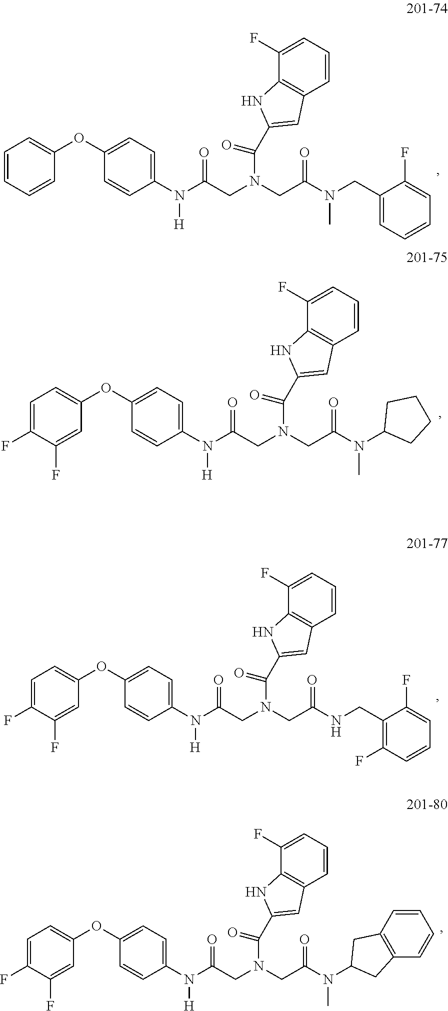 [SMILES: CN(Cc1ccccc1F)C(=O)CN(CC(=O)Nc1ccc(Oc2ccccc2)cc1)C(=O)c1cc2cccc(F)c2[nH]1 ; CN(C(=O)CN(CC(=O)Nc1ccc(Oc2ccc(F)c(F)c2)cc1)C(=O)c1cc2cccc(F)c2[nH]1)C1CCCC1 ; O=C(CN(CC(=O)Nc1ccc(Oc2ccc(F)c(F)c2)cc1)C(=O)c1cc2cccc(F)c2[nH]1)NCc1c(F)cccc1F ; CN(C(=O)CN(CC(=O)Nc1ccc(Oc2ccc(F)c(F)c2)cc1)C(=O)c1cc2cccc(F)c2[nH]1)C1Cc2ccccc2C1]

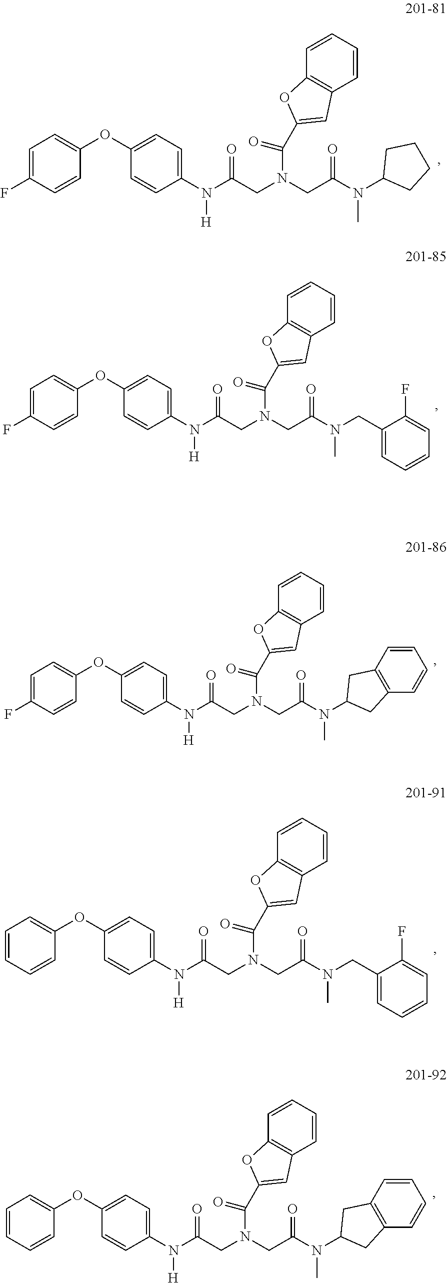 [SMILES: CN(C(=O)CN(CC(=O)Nc1ccc(Oc2ccc(F)cc2)cc1)C(=O)c1cc2ccccc2o1)C1CCCC1 ; CN(Cc1ccccc1F)C(=O)CN(CC(=O)Nc1ccc(Oc2ccc(F)cc2)cc1)C(=O)c1cc2ccccc2o1 ; CN(C(=O)CN(CC(=O)Nc1ccc(Oc2ccc(F)cc2)cc1)C(=O)c1cc2ccccc2o1)C1Cc2ccccc2C1 ; CN(Cc1ccccc1F)C(=O)CN(CC(=O)Nc1ccc(Oc2ccccc2)cc1)C(=O)c1cc2ccccc2o1 ; CN(C(=O)CN(CC(=O)Nc1ccc(Oc2ccccc2)cc1)C(=O)c1cc2ccccc2o1)C1Cc2ccccc2C1]

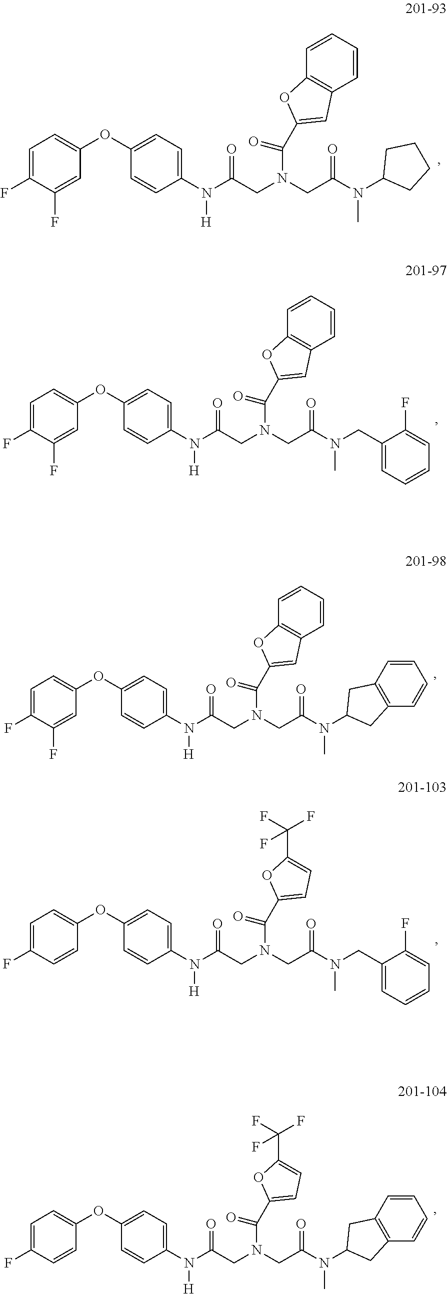 [SMILES: CN(C(=O)CN(CC(=O)Nc1ccc(Oc2ccc(F)c(F)c2)cc1)C(=O)c1cc2ccccc2o1)C1CCCC1 ; CN(Cc1ccccc1F)C(=O)CN(CC(=O)Nc1ccc(Oc2ccc(F)c(F)c2)cc1)C(=O)c1cc2ccccc2o1 ; CN(C(=O)CN(CC(=O)Nc1ccc(Oc2ccc(F)c(F)c2)cc1)C(=O)c1cc2ccccc2o1)C1Cc2ccccc2C1 ; CN(Cc1ccccc1F)C(=O)CN(CC(=O)Nc1ccc(Oc2ccc(F)cc2)cc1)C(=O)c1ccc(C(F)(F)F)o1 ; CN(C(=O)CN(CC(=O)Nc1ccc(Oc2ccc(F)cc2)cc1)C(=O)c1ccc(C(F)(F)F)o1)C1Cc2ccccc2C1]

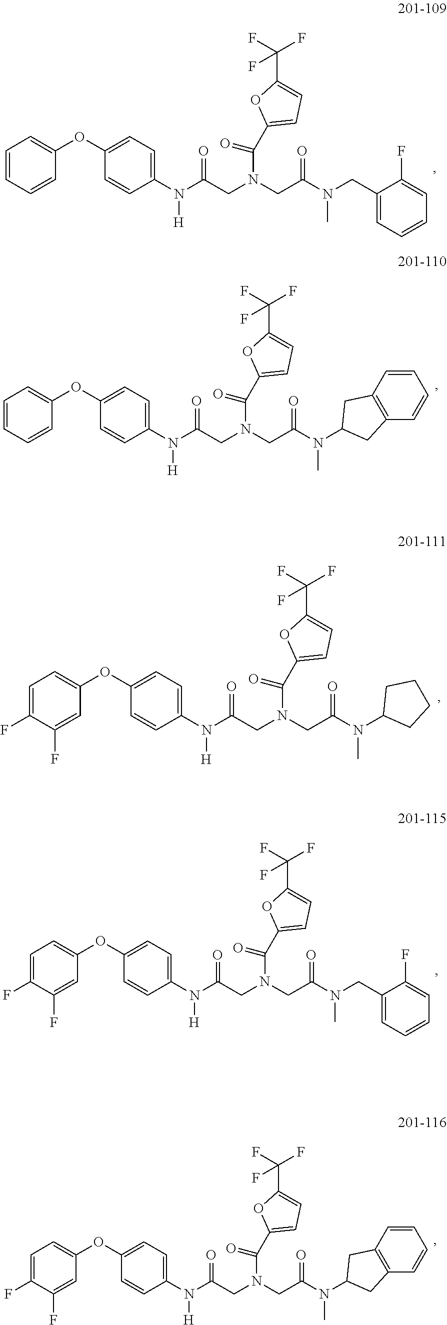 [SMILES: CN(Cc1ccccc1F)C(=O)CN(CC(=O)Nc1ccc(Oc2ccccc2)cc1)C(=O)c1ccc(C(F)(F)F)o1 ; CN(C(=O)CN(CC(=O)Nc1ccc(Oc2ccccc2)cc1)C(=O)c1ccc(C(F)(F)F)o1)C1Cc2ccccc2C1 ; CN(C(=O)CN(CC(=O)Nc1ccc(Oc2ccc(F)c(F)c2)cc1)C(=O)c1ccc(C(F)(F)F)o1)C1CCCC1 ; CN(Cc1ccccc1F)C(=O)CN(CC(=O)Nc1ccc(Oc2ccc(F)c(F)c2)cc1)C(=O)c1ccc(C(F)(F)F)o1 ; CN(C(=O)CN(CC(=O)Nc1ccc(Oc2ccc(F)c(F)c2)cc1)C(=O)c1ccc(C(F)(F)F)o1)C1Cc2ccccc2C1]

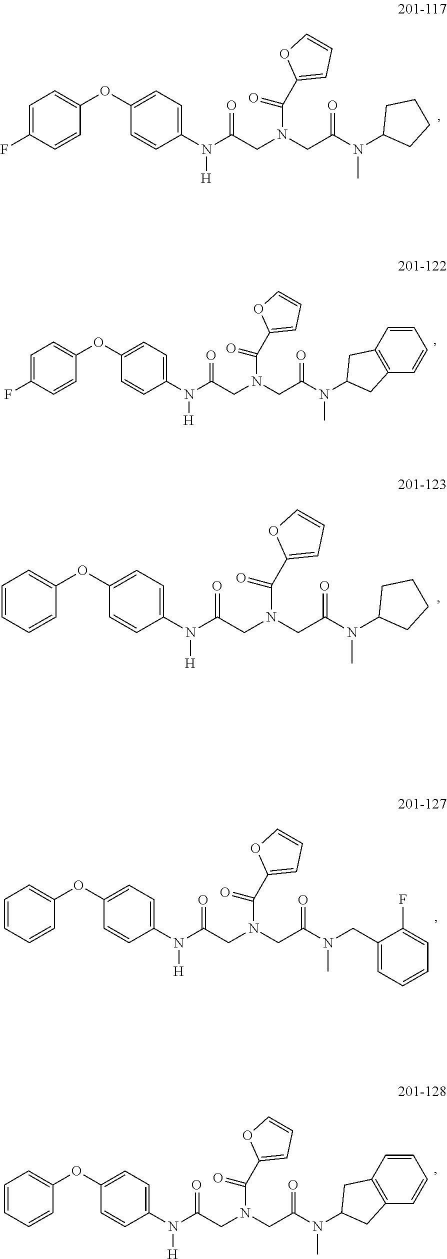 [SMILES: CN(C(=O)CN(CC(=O)Nc1ccc(Oc2ccc(F)cc2)cc1)C(=O)c1ccco1)C1CCCC1 ; CN(C(=O)CN(CC(=O)Nc1ccc(Oc2ccc(F)cc2)cc1)C(=O)c1ccco1)C1Cc2ccccc2C1 ; CN(C(=O)CN(CC(=O)Nc1ccc(Oc2ccccc2)cc1)C(=O)c1ccco1)C1CCCC1 ; CN(Cc1ccccc1F)C(=O)CN(CC(=O)Nc1ccc(Oc2ccccc2)cc1)C(=O)c1ccco1 ; CN(C(=O)CN(CC(=O)Nc1ccc(Oc2ccccc2)cc1)C(=O)c1ccco1)C1Cc2ccccc2C1]

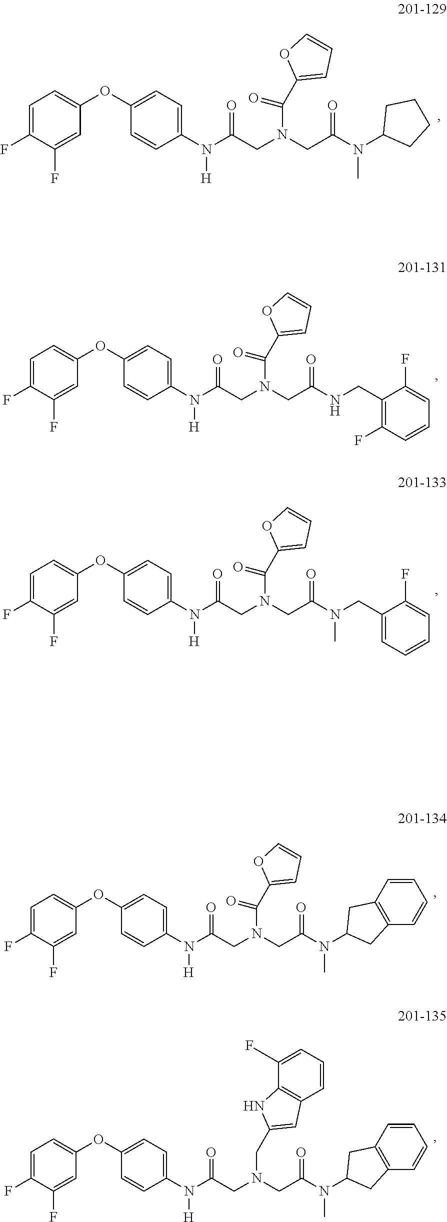 [SMILES: CN(C(=O)CN(CC(=O)Nc1ccc(Oc2ccc(F)c(F)c2)cc1)C(=O)c1ccco1)C1CCCC1 ; O=C(CN(CC(=O)Nc1ccc(Oc2ccc(F)c(F)c2)cc1)C(=O)c1ccco1)NCc1c(F)cccc1F ; CN(Cc1ccccc1F)C(=O)CN(CC(=O)Nc1ccc(Oc2ccc(F)c(F)c2)cc1)C(=O)c1ccco1 ; CN(C(=O)CN(CC(=O)Nc1ccc(Oc2ccc(F)c(F)c2)cc1)C(=O)c1ccco1)C1Cc2ccccc2C1 ; CN(C(=O)CN(CC(=O)Nc1ccc(Oc2ccc(F)c(F)c2)cc1)Cc1cc2cccc(F)c2[nH]1)C1Cc2ccccc2C1]

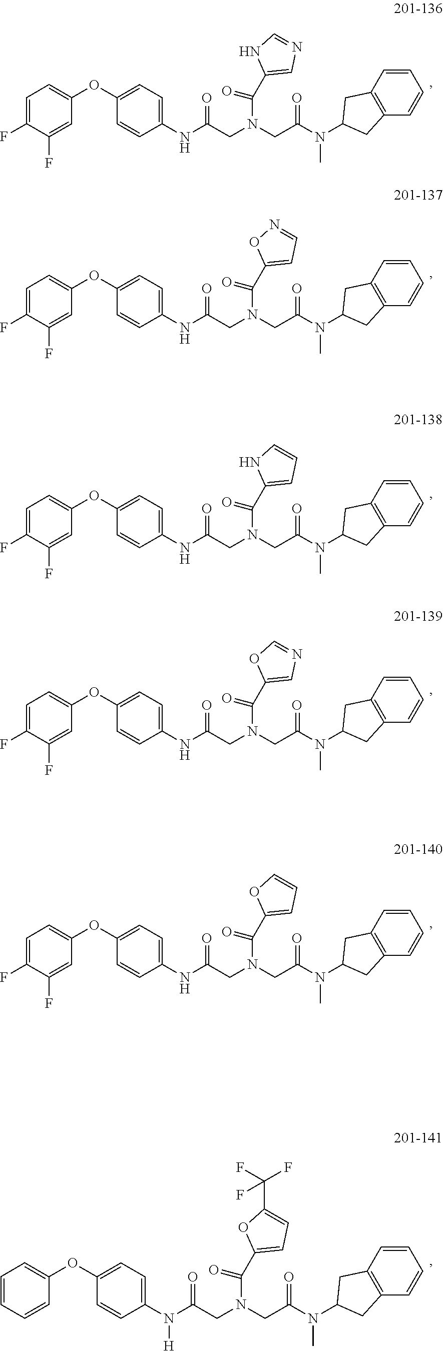 [SMILES: CN(C(=O)CN(CC(=O)Nc1ccc(Oc2ccc(F)c(F)c2)cc1)C(=O)c1cnc[nH]1)C1Cc2ccccc2C1 ; CN(C(=O)CN(CC(=O)Nc1ccc(Oc2ccc(F)c(F)c2)cc1)C(=O)c1ccno1)C1Cc2ccccc2C1 ; CN(C(=O)CN(CC(=O)Nc1ccc(Oc2ccc(F)c(F)c2)cc1)C(=O)c1ccc[nH]1)C1Cc2ccccc2C1 ; CN(C(=O)CN(CC(=O)Nc1ccc(Oc2ccc(F)c(F)c2)cc1)C(=O)c1cnco1)C1Cc2ccccc2C1 ; CN(C(=O)CN(CC(=O)Nc1ccc(Oc2ccc(F)c(F)c2)cc1)C(=O)c1ccco1)C1Cc2ccccc2C1 ; CN(C(=O)CN(CC(=O)Nc1ccc(Oc2ccccc2)cc1)C(=O)c1ccc(C(F)(F)F)o1)C1Cc2ccccc2C1]

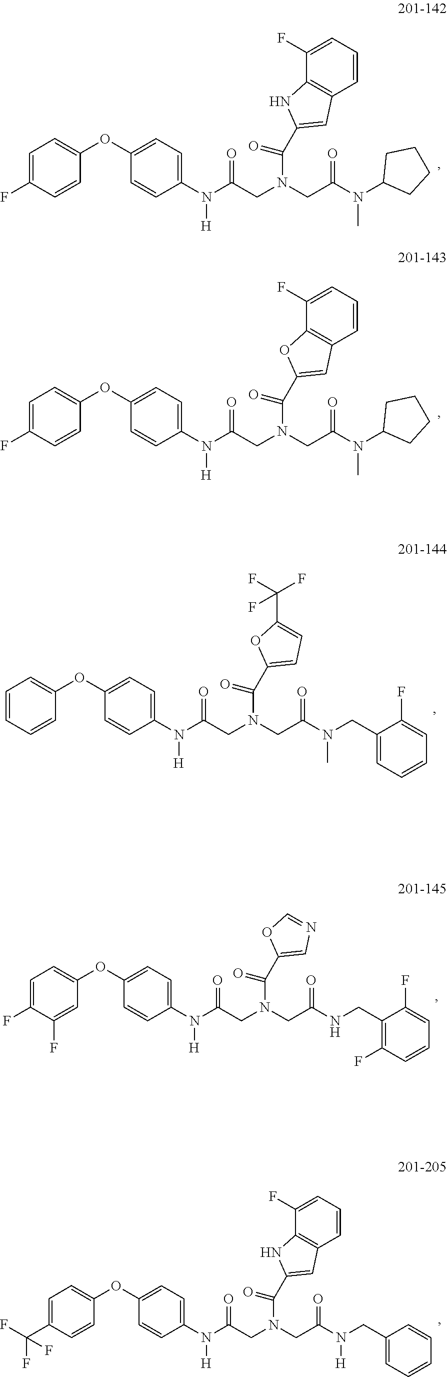 [SMILES: CN(C(=O)CN(CC(=O)Nc1ccc(Oc2ccc(F)cc2)cc1)C(=O)c1cc2cccc(F)c2[nH]1)C1CCCC1 ; CN(C(=O)CN(CC(=O)Nc1ccc(Oc2ccc(F)cc2)cc1)C(=O)c1cc2cccc(F)c2o1)C1CCCC1 ; CN(Cc1ccccc1F)C(=O)CN(CC(=O)Nc1ccc(Oc2ccccc2)cc1)C(=O)c1ccc(C(F)(F)F)o1 ; O=C(CN(CC(=O)Nc1ccc(Oc2ccc(F)c(F)c2)cc1)C(=O)c1cnco1)NCc1c(F)cccc1F ; O=C(CN(CC(=O)Nc1ccc(Oc2ccc(C(F)(F)F)cc2)cc1)C(=O)c1cc2cccc(F)c2[nH]1)NCc1ccccc1]

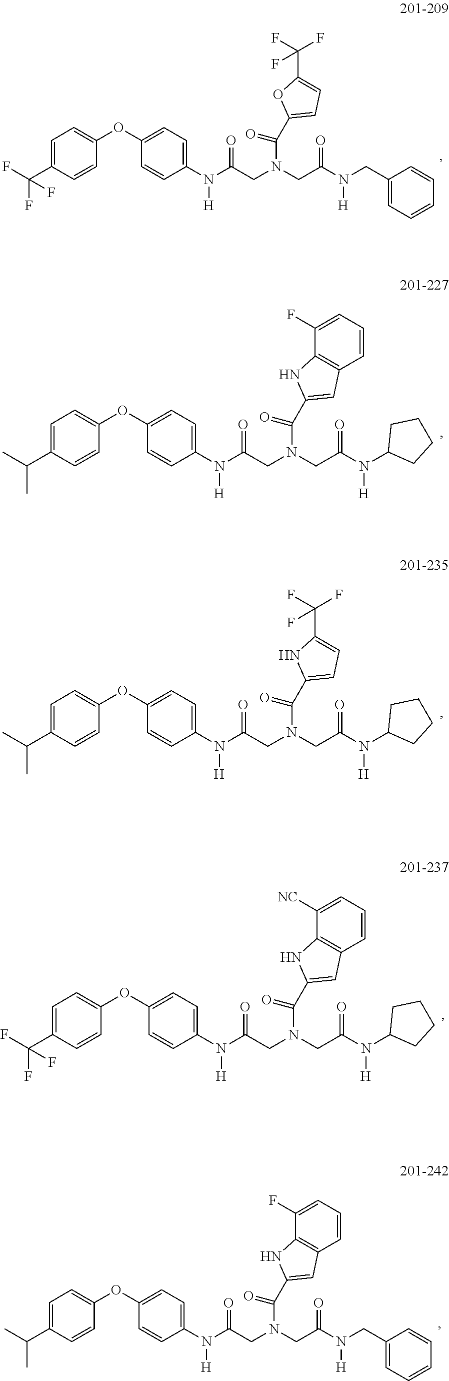 [SMILES: O=C(CN(CC(=O)Nc1ccc(Oc2ccc(C(F)(F)F)cc2)cc1)C(=O)c1ccc(C(F)(F)F)o1)NCc1ccccc1 ; CC(C)c1ccc(Oc2ccc(NC(=O)CN(CC(=O)NC3CCCC3)C(=O)c3cc4cccc(F)c4[nH]3)cc2)cc1 ; CC(C)c1ccc(Oc2ccc(NC(=O)CN(CC(=O)NC3CCCC3)C(=O)c3ccc(C(F)(F)F)[nH]3)cc2)cc1 ; N#Cc1cccc2cc(C(=O)N(CC(=O)Nc3ccc(Oc4ccc(C(F)(F)F)cc4)cc3)CC(=O)NC3CCCC3)[nH]c12 ; CC(C)c1ccc(Oc2ccc(NC(=O)CN(CC(=O)NCc3ccccc3)C(=O)c3cc4cccc(F)c4[nH]3)cc2)cc1]

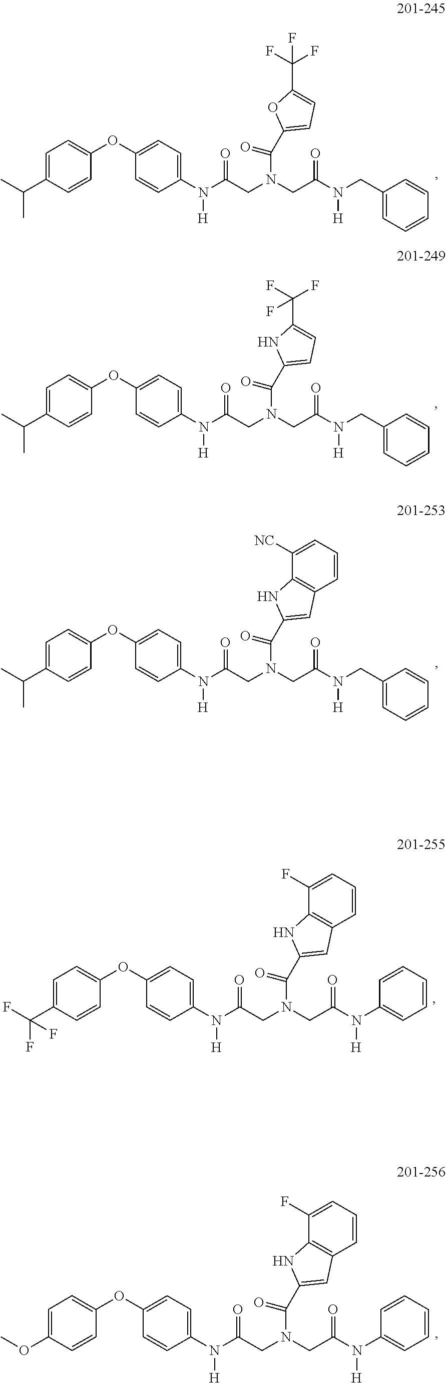 [SMILES: CC(C)c1ccc(Oc2ccc(NC(=O)CN(CC(=O)NCc3ccccc3)C(=O)c3ccc(C(F)(F)F)o3)cc2)cc1 ; CC(C)c1ccc(Oc2ccc(NC(=O)CN(CC(=O)NCc3ccccc3)C(=O)c3ccc(C(F)(F)F)[nH]3)cc2)cc1 ; CC(C)c1ccc(Oc2ccc(NC(=O)CN(CC(=O)NCc3ccccc3)C(=O)c3cc4cccc(C#N)c4[nH]3)cc2)cc1 ; O=C(CN(CC(=O)Nc1ccc(Oc2ccc(C(F)(F)F)cc2)cc1)C(=O)c1cc2cccc(F)c2[nH]1)Nc1ccccc1 ; COc1ccc(Oc2ccc(NC(=O)CN(CC(=O)Nc3ccccc3)C(=O)c3cc4cccc(F)c4[nH]3)cc2)cc1]

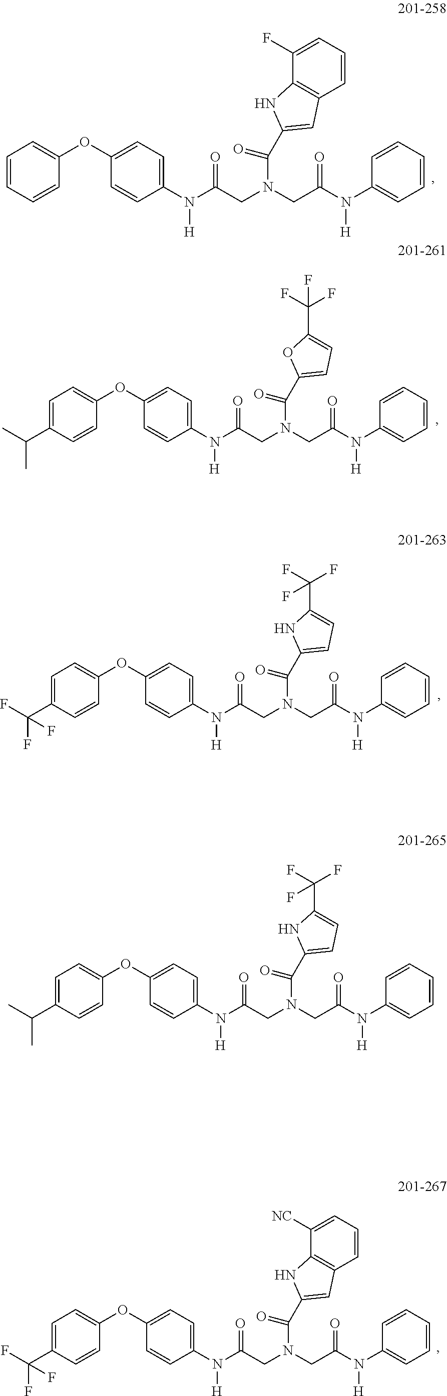 [SMILES: O=C(CN(CC(=O)Nc1ccc(Oc2ccccc2)cc1)C(=O)c1cc2cccc(F)c2[nH]1)Nc1ccccc1 ; CC(C)c1ccc(Oc2ccc(NC(=O)CN(CC(=O)Nc3ccccc3)C(=O)c3ccc(C(F)(F)F)o3)cc2)cc1 ; O=C(CN(CC(=O)Nc1ccc(Oc2ccc(C(F)(F)F)cc2)cc1)C(=O)c1ccc(C(F)(F)F)[nH]1)Nc1ccccc1 ; CC(C)c1ccc(Oc2ccc(NC(=O)CN(CC(=O)Nc3ccccc3)C(=O)c3ccc(C(F)(F)F)[nH]3)cc2)cc1 ; N#Cc1cccc2cc(C(=O)N(CC(=O)Nc3ccccc3)CC(=O)Nc3ccc(Oc4ccc(C(F)(F)F)cc4)cc3)[nH]c12]

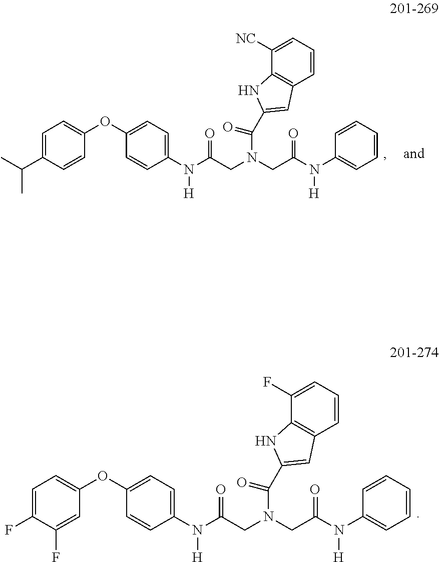 [SMILES: CC(C)c1ccc(Oc2ccc(NC(=O)CN(CC(=O)Nc3ccccc3)C(=O)c3cc4cccc(C#N)c4[nH]3)cc2)cc1 ; O=C(CN(CC(=O)Nc1ccc(Oc2ccc(F)c(F)c2)cc1)C(=O)c1cc2cccc(F)c2[nH]1)Nc1ccccc1]

### Pharmaceutical Compositions, Formulations and Kits

In another aspect, the present disclosure provides a pharmaceutical composition comprising a compound of the present disclosure (also referred to as the “active ingredient”) and pharmaceutically acceptable excipients. In some embodiments, the pharmaceutical composition comprises an effective amount of the compound of the present disclosure. In some embodiments, the pharmaceutical composition comprises a therapeutically effective amount of the compound of the present disclosure. In some embodiments, the pharmaceutical composition comprises a prophylactically effective amount of the compound of the present disclosure.

Pharmaceutically acceptable excipients for use in the present disclosure refer to the non-toxic carriers, adjuvants or vehicles, which do not destroy the pharmacological activity of the compounds formulated together. Pharmaceutically acceptable carriers, adjuvants, or vehicles that can be used in the compositions of the present disclosure include (but are not limited to) ion exchangers, alumina, aluminum stearate, lecithin, serum proteins (such as human serum proteins), buffer substances (such as phosphate), glycine, sorbic acid, potassium sorbate, mixture of partial glycerides of saturated plant fatty acids, water, salts or electrolytes (such as protamine sulfate), disodium hydrogen phosphate, potassium hydrogen phosphate, sodium chloride, zinc salts, silica gel, magnesium trisilicate, polyvinyl pyrrolidone, cellulose-based substance, polyethylene glycol, sodium carboxymethyl cellulose, polyacrylate, wax, polyethylene-polyoxypropylene block polymers, polyethylene glycol and lanolin.

The present disclosure also includes kits (e.g., pharmaceutical packs). The kits provided may include a compound of the present disclosure, other therapeutic agent(s), and a first and a second containers (e.g., vials, ampoules, bottles, syringes, and/or dispersible packages or other suitable containers) containing the compound of the present disclosure and other therapeutic agent(s). In some embodiments, the provided kits can also optionally include a third container containing a pharmaceutically acceptable excipient for diluting or suspending the compound of the present disclosure and/or other therapeutic agent(s). In some embodiments, the compound of the present disclosure provided in the first container and other therapeutic agent(s) provided in the second container are combined to form a unit dosage form.

Administration

The pharmaceutical composition provided by the present disclosure can be administered by a variety of routes including, but not limited to, oral administration, parenteral administration, inhalation administration, topical administration, rectal administration, nasal administration, buccal administration, vaginal administration, administration by implant or other means of administration. For example, parenteral administration as used herein includes subcutaneous administration, intradermal administration, intravenous administration, intramuscular administration, intra-articular administration, intra-arterial administration, intrasynovial administration, intrasternal administration, intracerebroventricular administration, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the route of administration selected, the actual compound administered, the age, weight and response of the individual patient, the severity of the patient's symptoms, etc.

When used to prevent the conditions described in the present disclosure, the compounds provided herein will be administered to a subject at risk of developing the conditions, typically based on the physician's recommendation and administered under the supervision of the physician, at the dosage level described above. Subjects at risk of developing the particular conditions generally include those who have a family history of the conditions, or those who have been identified by genetic testing or screening to be particularly susceptible to developing the conditions.

The pharmaceutical compositions provided herein can also be administered chronically (“chronic administration”). Chronic administration refers to the administration of a compound or pharmaceutical composition thereof for a long period of time, for example, 3 months, 6 months, 1 year, 2 years, 3 years, 5 years, etc., or can be continuously administered indefinitely, for example, for the rest of the subject's life. In some embodiments, the chronic administration is intended to provide a constant level of the said compound in the blood over a long period of time, for example, within the therapeutic window.

Pharmaceutical compositions of the present disclosure can be further delivered using various dosing methods. For example, in some embodiments, pharmaceutical compositions can be administered by bolus injection, for example, to increase the concentration of the compound in the blood to an effective level. The bolus dose depends on the desired systemic level of the active ingredient throughout the body, for example, intramuscular or subcutaneous bolus dose allows a slow release of the active ingredient, while the bolus that is delivered directly to the vein (e.g., via IV intravenous drip) allows a much faster delivery which quickly raises the concentration of the active ingredient in the blood to an effective level. In other embodiments, pharmaceutical compositions can be administered in a form of continuous infusion, for example, via IV intravenous drip, thereby providing a steady state concentration of the active ingredient in the subject's body.

Moreover, in other embodiments, a bolus dose of the pharmaceutical compositions can be administered first, followed by continuous infusion.

The compositions for oral administration can be in the form of bulk liquid solution or suspension or bulk powder. More commonly, however, in order to facilitate the precise dosing, the compositions are provided in unit dosage form. The term “unit dosage form” refers to physical discrete units suitable as unitary dosages for human patients and other mammals, each unit containing a predetermined quantity of active ingredient calculated to produce the desired therapeutic effects with suitable pharmaceutical excipients. Typical unit dosage forms include prefilled, pre-measured ampoules or syringes of the liquid compositions, or pills, tablets, capsules, etc. in the case of solid compositions. In such compositions, the said compound generally will be the minor component (about 0.1 to about 50% by weight, or preferably about 1 to about 40% by weight), with the remainder being various carriers or excipients and processing aids useful for forming the desired dosing form.

For oral dosage, a representative scheme is one to five, especially two to four, and typically three oral doses per day. Using these dosing patterns, each dose provides from about 0.01 to about 20 mg/kg of the compound of the present disclosure, with preferred doses each providing from about 0.1 to about 10 mg/kg, and especially from about 1 to about 5 mg/kg.

Transdermal doses are generally selected to provide similar or lower blood levels than are achieved using injection doses, usually in an amount of from about 0.01 to about 20% by weight, preferably from about 0.1 to about 20% by weight, preferably from about 0.1 to about 10% by weight, and more preferably from about 0.5 to about 15% by weight.

The injection dose level ranges from about 0.1 mg/kg/hr to at least 10 mg/kg/hr, all for from about 1 to about 120 hours, especially from 24 to 96 hours. In order to achieve a sufficient level of steady state, a preloading bolus of from about 0.1 mg/kg to about 10 mg/kg or more can also be administered. For human patients of 40 to 80 kg, the maximum total dose should not exceed approximately 2 g/day.

Liquid forms suitable for oral administration may include suitable aqueous or nonaqueous carriers, buffers, suspending agents and dispersants, coloring agents, flavoring agents, etc. Solid forms may include, for example, any of the following components, or compounds having the similar properties: binders, for example, microcrystalline cellulose, tragacanth gum or gelatin; excipients, for example, starch or lactose; disintegrants, for example, alginic acid, Primogel or corn starch; lubricants, for example, magnesium stearate; glidants, for example, colloidal silica; sweeteners, for example, sucrose or saccharin; or flavoring agents, for example, peppermint, methyl salicylate or orange flavouring.

Injectable compositions are typically based on the injectable sterile saline or phosphate-buffered saline, or other injectable excipients known in the art. As previously mentioned, in such compositions, the active ingredients will typically be the minor component, often from about 0.05 to 10% by weight, with the remainder being injectable excipients, etc.

The transdermal compositions are typically formulated as topical ointments or creams containing the active ingredients. When formulated as an ointment, the active ingredients are typically combined with paraffin or water miscible ointment base. Alternatively, the active ingredients can be formulated as a cream with, for example, oil-in-water cream base. Such transdermal formulations are well-known in the art and generally include other ingredients for enhancing stable skin penetration of the active ingredients or the formulations. All such known transdermal formulations and components are included within the scope of the present disclosure.

The compounds of the present disclosure may also be administered by transdermal devices. Thus, transdermal administration can be accomplished using a patch either of reservoir or porous membrane type, or of a plurality of solid substrates.

The above components of the compositions for oral administration, injection or topical administration are only representative. Other materials and processing techniques, etc., are described in the Section 8 of Remington's Pharmaceutical Sciences, 17th edition, 1985, Mack Publishing Company, Easton, Pa., which is incorporated herein by reference.

Compounds of the present disclosure may also be administered in a sustained release form or from a sustained release delivery system. Description of the representative sustained release materials can be found in Remington's Pharmaceutical Sciences.

The present disclosure also relates to pharmaceutically acceptable formulations of the compounds of the present disclosure. In one embodiment, the formulation comprises water. In another embodiment, the formulation comprises cyclodextrin derivative. The most common cyclodextrins are alpha-, beta- and gamma-cyclodextrins consisting of 6, 7 and 8 alpha-1,4-linked glucose units, respectively, optionally including one or more substituents on the linked sugar moiety, including, but are not limited to, methylated, hydroxyalkylated, acylated, and sulfoalkyl ether substitution. In some embodiments, the cyclodextrin is sulfoalkyl ether beta-cyclodextrin, e.g., sulfobutyl ether beta-cyclodextrin, also known as Captisol. See, for example, U.S. Pat. No. 5,376,645. In some embodiments, the formulation comprises hexapropyl-D-cyclodextrin (e.g., 10-50% in water).

Combination Therapy

The compounds disclosed herein may also be administered with other therapeutic agents such as cholesterol-lowering agents, fibrates and hypolipidemic agents, anti-diabetic agents, antihypertensive agents and angiotensin-converting-enzyme (ACE) inhibitors.

In some embodiments, the other therapeutic agent is a cholesterol-lowering agents. Non limiting examples of cholesterol-lowering agents are atorvastatin, cerivastatin, fluvastatin, lovastatin, pitavastatin, pravastatin, rosuvastatin, simvastatin, ezetimibe, and the combination of ezetimibe/simvastatin (Vytorin®).

In some embodiments, the other therapeutic agent is a fibrate or hypolipidemic agent. Non-limiting examples of fibrates or hypolipidemic agents are acifran, acipimox, beclobrate, bezafibrate, binifibrate, ciprofibrate, clofibrate, colesevelam, gemfibrozil, fenofibrate, melinamide, niacin, and ronafibrate.

In some embodiments, the other therapeutic agent is a DPP-IV inhibitor as anti-diabetic agent. Non-limiting examples of DPP-IV inhibitors as anti-diabetic agents are sitagliptin, saxagliptin, vildagliptin, linagliptin, dutogliptin, gemigliptin and alogliptin.

In some embodiments, the other therapeutic agent is an anti-diabetic agent other than a DPP-IV inhibitor. Non-limiting examples of anti-diabetic agents are acarbose, epalrestat, exenatide, glimepiride, liraglutide, metformin, miglitol, mitiglinide, nateglinide, pioglitazone, pramlintide, repaglinide, rosiglitazone, tolrestat, troglitazone, and voglibose.

In some embodiments, the other therapeutic agent is an antihypertensive agents. Non-limiting examples of antihypertensive agents include alacepril, alfuzosin, aliskiren, amlodipine besylate, amosulalol, aranidipine, arotinolol HCl, azelnidipine, bamidipine hydrochloride, benazepril hydrochloride, benidipine hydrochloride, betaxolol HCl, bevantolol HCl, bisoprolol fumarate, bopindolol, bosentan, budralazine, bunazosin HCl, candesartan cilexetil, captopril, carvedilol, celiprolol HCl, cicletanine, cilazapril, cinildipine, clevidipine, delapril, dilevalol, doxazosin mesylate, efonidipine, enalapril maleate, enalaprilat, eplerenone, eprosartan, felodipine, fenoldopam mesylate, fosinopril sodium, guanadrel sulfate, imidapril HCl, irbesartan, isradipine, ketanserin, lacidipine, lercanidipine, lisinopril, losartan, manidipine hydrochloride, mebeffadil hydrochloride, moxonidine, nebivolol, nilvadipine, nipradilol, nisoldipine, olmesartan medoxomil, perindopril, pinacidil, quinapril, ramipril, rilmedidine, spirapril HCl, telmisartan, temocarpil, terazosin HCl, tertatolol HCl, tiamenidine HCl, tilisolol hydrochloride, trandolapril, treprostinil sodium, trimazosin HCl, valsartan, and zofenopril calcium.

In other embodiments, suitable angiotensin-converting-enzyme (ACE) inhibitors used in the above-described combination therapies include, without limitation, enalapril, ramipril, quinapril, perindopril, lisinopril, imidapril, zofenopril, trandolapril, fosinopril, and captopril.

### EXAMPLES

The following examples are provided to provide those skilled in the art with a complete disclosure and description of how to implement, prepare and evaluate the methods and compounds claimed herein, and are intended to be illustrative only and not limiting the scope of the invention.

The preparation protocols of the compounds disclosed herein are shown below.

### Scheme 1: General Synthetic Schemes for the Preparations of Monoamide Intermediates

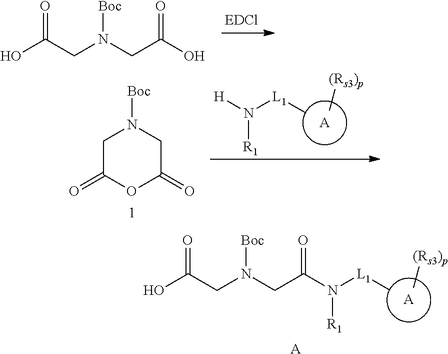

According to Scheme 1, in situ closure of N-BOC-iminodiacetic acid to the anhydride 1 (1 equiv EDCI, in DMF), followed by treatment with amines (1 equiv, in DMF) afforded the monoamides A.

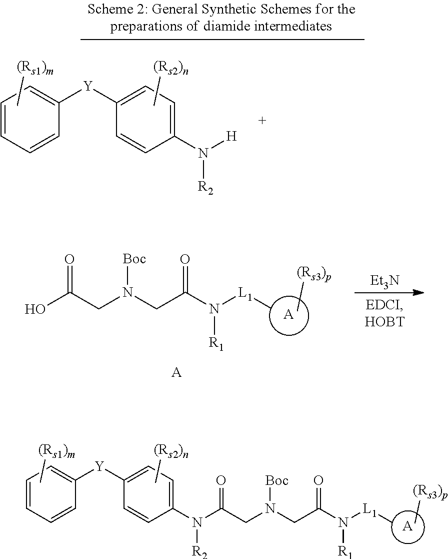

According to Scheme 2, monoamides A were treated with amines, EDCI, and HOBT to afford the diamide intermediates B.

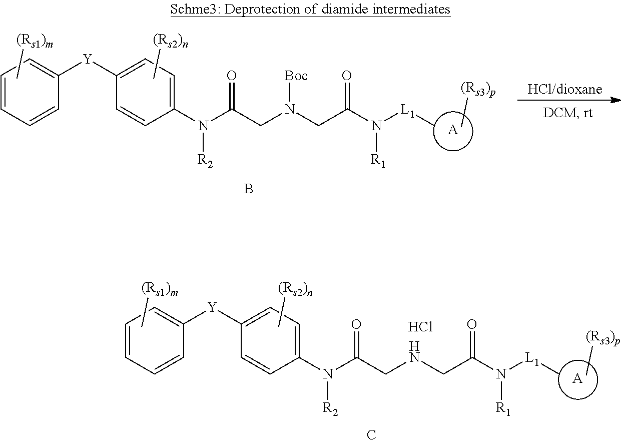

According to Scheme 3, N-Boc deprotection of diamides B using 4 N HCl-dioxane at room temperature afforded the HCl salt of amines C.

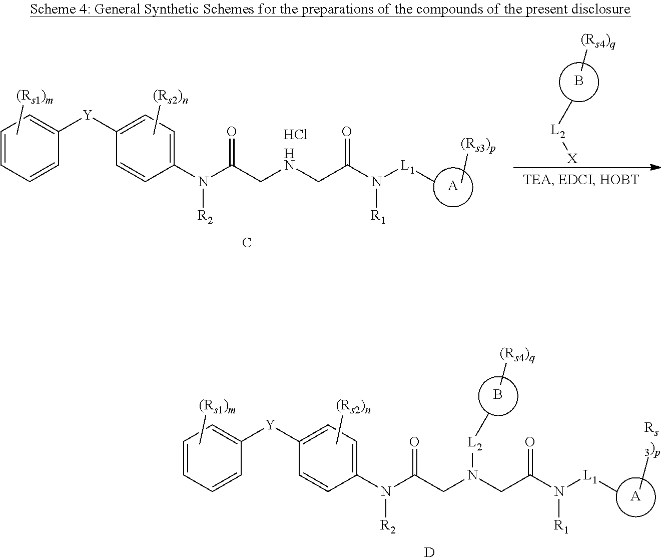

According to Scheme 4, compounds of the present disclosure can be prepared from the reaction of diamide intermediates with a compound, wherein X could be a halogen, aldehyde and carboxylic acid, in the presence of EDCI, and HOBT.

### General Synthetic Procedure for the Preparations of Monoamide Intermediates

A solution of N-((tert-butyloxy)carbonyl)iminodiacetic acid (2.33 g, 10 mmol) in DCM (30 mL) was treated with EDCI (1.98 g, 10.3 mmol) at 25° C. The mixture was stirred at 25° C. for 1 h before the amine (12 mmol) was added, and the solution was stirred for 20 h at 25° C. The reaction mixture was poured into 10% HCl_{(aq)} (100 ml) and extracted with DCM (100 ml×2). The organic phase was washed with 10% HCl_{(aq)} (80 ml×2), and Sat. NaCl_{(aq)} (100 ml×2), dried (MgSO₄), filtered, and concentrated in vacuo to yield the pure N-((tert-butyloxy)-carbonyl)iminodiacetic acid monoamide.

### General Procedure for the Preparations of Diamide Intermediates

The N-((tert-butyloxy)-carbonyl)iminodiacetic acid monoamide (4.8 mmol) was dissolved in DCM (15 ml). The solution was treated with amine (1 equiv), EDCI (1.2 equiv), HOBt (1.2 equiv) and Et₃N (1.5 equiv). The solution was stirred at 25° C. for 20 h. The mixture was poured into H₂O and extracted with DCM (40 ml×2). The organic phase was washed with Sat. NaCl_{(aq)} (50 ml×2), dried (MgSO₄), filtered, and concentrated in vacuo. The crude was purified by MPLC to yield the pure diamides.

### General Procedures for the Preparations of Boc Deprotection Diamide Intermediates

The N′-((tert-butyloxy)carbonyl)-N,N-disubstituted iminodiacetic acid diamide (2.88 mmol) was dissolved in 4N HCl-dioxane, and the mixture was stirred at 25° C. for 1 h. The solvent was removed under vacuum. The residue was purified by MPLC to furnish the desired products.

According to the above procedures, the following diamide intermediates are made:

| Compound | | |
|---|---|---|
| No. | Structure | Identification data |
| B5a | 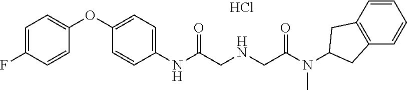 [SMILES: CN(C(=O)CNCC(=O)Nc1ccc(Oc2ccc(F)cc2)cc1)C1Cc2ccccc2C1 ; Cl] | ¹H NMR (500 MHz, DMSO): δ 2.94-3.21 (m, 4H), 2.80 (s, 3H), 3.97-3.99 (m, 2H), 4.13 (s, 1H), 4.31 (s, 1H), 4.59-5.37 (m, 1H), 7.00-7.68 (m, 12H), 9.22 (bs, 2H), 10.92-10.94 (m, 1H), chemical formula: C₂₆H₂₆FN₃O₃ m/z Calcd for [M + H]⁺ 448.2, m/z found 448.2. |
| B7a | 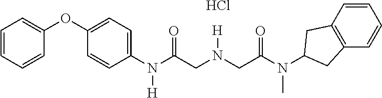 [SMILES: CN(C(=O)CNCC(=O)Nc1ccc(Oc2ccccc2)cc1)C1Cc2ccccc2C1 ; Cl] | ¹H NMR (500 MHz, DMSO): δ 2.80 (s, 3H), 2.94-3.21 (m, 4H), 3.98 (bs, 2H), 4.13 (bs, 1H), 4.31 (s, 1H), 4.58-5.38 (m, 1H), 6.96-7.68 (m, 13H), 9.24 (bs, 2H), 10.96-10.98 (m, 1H). |

### General Procedure for the Preparations of Compounds of the Present Disclosure

A solution of diamide (0.25 mmol), carboxylic acid (0.25 mmol), EDCI (0.30 mol), HOBt (0.3 mol), Et₃N (0.05 ml), DMF (2 ml) was stirred for 20 hr at 25° C. The mixture was poured into 10% HCl(aq.) and extracted with EtOAc. The organic phase was washed with Sat. NaCl(aq). The organic phase was dried (MgSO₄), filtered, and concentrated in vacuo. The residue was purified by MPLC to furnish the desired products.

### Preparation of compound 201-29

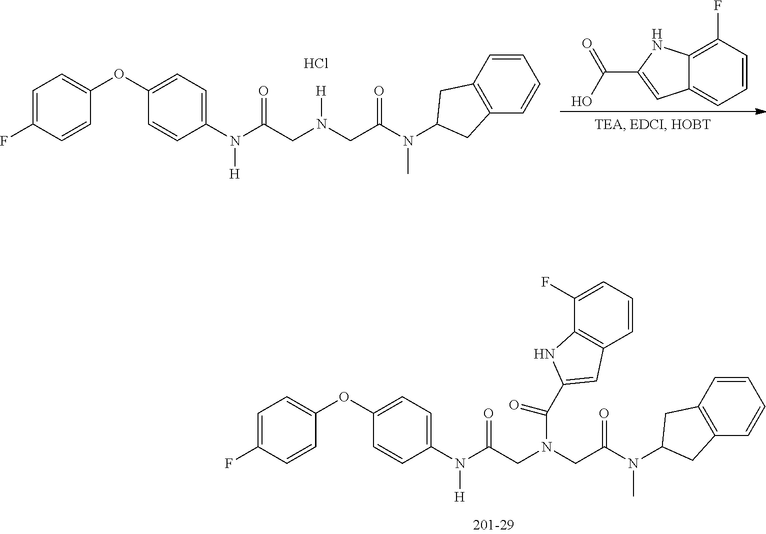 [SMILES: CN(C(=O)CNCC(=O)Nc1ccc(Oc2ccc(F)cc2)cc1)C1Cc2ccccc2C1 ; Cl ; O=C(O)c1cc2cccc(F)c2[nH]1 ; CN(C(=O)CN(CC(=O)Nc1ccc(Oc2ccc(F)cc2)cc1)C(=O)c1cc2cccc(F)c2[nH]1)C1Cc2ccccc2C1]

Compound 201-29 was prepared using the general procedure for the preparations of compounds of the present disclosure. Particularly, a solution of diamide B5a (112 mg, 0.25 mmole), EDCI (47 mg, 0.30 mmole), HOBT (40 mg, 0.3 mmole), and Et₃N (0.05 ml) in DMF (2 ml) was stirred for 20 h at 25° C. The mixture was poured into 10% aq HCl (10 mL) and extracted with EtOAc (15 mL). The organic phase was washed with Sat. aq NaCl (2×10 mL). The organic phase was dried (MgSO₄), filtered, and concentrated in vacuo. The residue was purified by MPLC to furnish the desire product (129 mg, 85%).

Some other compounds were prepared according to the synthetic procedure of the compound 201-29 but using different carboxylic acids and diamide intermediates.

TABLE 1

| Compound | Carboxylic | | |
|---|---|---|---|
| No. | acid | Final compound | Identification data |
| 201-25 | 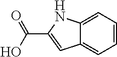 | 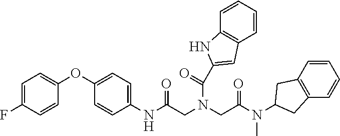 [SMILES: CN(C(=O)CN(CC(=O)Nc1ccc(Oc2ccc(F)cc2)cc1)C(=O)c1cc2ccccc2[nH]1)C1Cc2ccccc2C1] | ¹H NMR (500 MHz, DMSO): δ 2.79-3.22 (m, 7H), 4.26 (s, 1H), 4.49-5.39 (m, 4H), 6.62-7.64 (m, 17H), 10.78-11.09 (m, 1H), 11.66 (s, 1H), chemical formula: C₃₅H₃₁FN₄O₄ m/z Calcd for [M + H]⁺ 591.2, m/z found 591.3. |
| 201-29 | 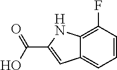 | 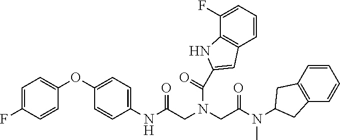 [SMILES: CN(C(=O)CN(CC(=O)Nc1ccc(Oc2ccc(F)cc2)cc1)C(=O)c1cc2cccc(F)c2[nH]1)C1Cc2ccccc2C1] | ¹H NMR (500 MHz, DMSO): δ 2.49-3.33 (m, 7H), 4.27 (s, 1H), 4.47-5.40 (m, 4H), 6.71- 7.65 (m, 16H), 10.77-10.98 (m. 1H), 12.16 (s, 1H), chemical formula: C₃₅H₃₀F₂N₄O₄ m/z Calcd for [M + H]₊ 609.2, m/z found 609.4. |
| 201-31 | 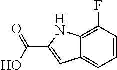 | 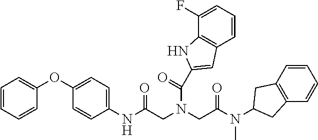 [SMILES: CN(C(=O)CN(CC(=O)Nc1ccc(Oc2ccccc2)cc1)C(=O)c1cc2cccc(F)c2[nH]1)C1Cc2ccccc2C1] | ¹H NMR (500 MHz, DMSO): δ 2.49-3.34 (m, 7H), 4.28 (s, 1H), 4.47-5.40 (m, 4H), 6.71- 7.70 (m, 17H), 10.82-11.04 (m, 1H), 12.16 (s, 1H), chemical formula: C₃₅H₃₁FN₄O₄ m/z Calcd for [M + H]⁺ 591.2, m/z found 591.4. |
| 201-80 | 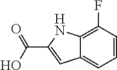 | 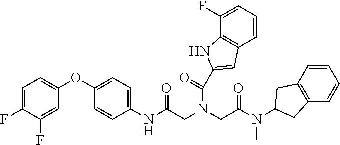 [SMILES: CN(C(=O)CN(CC(=O)Nc1ccc(Oc2ccc(F)c(F)c2)cc1)C(=O)c1cc2cccc(F)c2[nH]1)C1Cc2ccccc2C1] | ¹H NMR (500 MHz, DMSO): δ 2.80-3.29 (m, 7H), 4.29 (s, 1H), 4.49-5.45 (m, 4H), 6.73-7.73 (m, 15H), 10.84-11.03 (m, 1H), 12.22 (s, 1H), chemical formula: C₃₅H₂₉F₃N₄O₄ m/z Calcd for [M + H]⁺ 627.3, m/z found 627.22. |
| 201-86 | 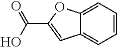 | 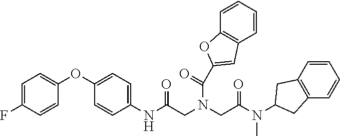 [SMILES: CN(C(=O)CN(CC(=O)Nc1ccc(Oc2ccc(F)cc2)cc1)C(=O)c1cc2ccccc2o1)C1Cc2ccccc2C1] | ¹H NMR (500 MHz, DMSO): δ 2.79-3.28 (m, 7H), 4.29-5.41 (m, 5H), 6.98-7.83 (m, 17H), 10.77-10.82 (m, 1H), chemical formula: C₃₅H₃₀FN₃O₅ m/z Calcd for [M + H]⁺ 592.22, m/z found 592.2. |
| 201-92 | 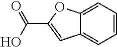 | 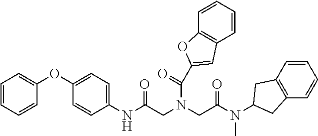 [SMILES: CN(C(=O)CN(CC(=O)Nc1ccc(Oc2ccccc2)cc1)C(=O)c1cc2ccccc2o1)C1Cc2ccccc2C1] | ¹H NMR (500 MHz, DMSO): δ 2.77-3.25 (m, 7H), 4.28-5.36 (m, 5H), 6.94-7.81 (m, 18H), 10.74-10.79 (m, 1H), chemical formula: C₃₅H₃₁N₃O₅ m/z Calcd for [M + H]⁺ 574.23, m/z found 574.2. |
| 201-98 | 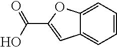 | 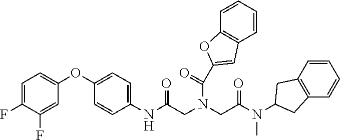 [SMILES: CN(C(=O)CN(CC(=O)Nc1ccc(Oc2ccc(F)c(F)c2)cc1)C(=O)c1cc2ccccc2o1)C1Cc2ccccc2C1] | ¹H NMR (500 MHz, DMSO): δ 2.78-3.20 (m, 7H), 4.28-5.36 (m, 5H), 6.77-7.81 (m, 16H), 10.78-10.83 (m, 1H), chemical formula: C₃₅H₂₉F₂N₃O₅ m/z Calcd for [M + H]⁺ 610.21, m/z found 610.3. |
| 201-104 | 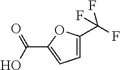 | 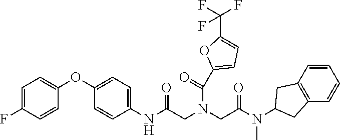 [SMILES: CN(C(=O)CN(CC(=O)Nc1ccc(Oc2ccc(F)cc2)cc1)C(=O)c1ccc(C(F)(F)F)o1)C1Cc2ccccc2C1] | ¹H NMR (500 MHz, DMSO): δ 2.77-3.26 (m, 7H), 4.25-5.38 (m, 5H), 6.80-7.64 (m, 14H), 10.62-10.73 (m, 1H), chemical formula: C₃₂H₂₇F₄N₃O₅ m/z Calcd for [M + H]⁺ 610.19, m/z found 610.2. |
| 201-110 | 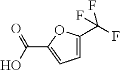 | 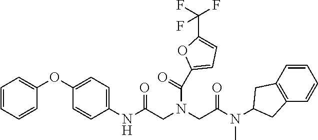 [SMILES: CN(C(=O)CN(CC(=O)Nc1ccc(Oc2ccccc2)cc1)C(=O)c1ccc(C(F)(F)F)o1)C1Cc2ccccc2C1] | ¹H NMR (500 MHz, DMSO): δ 2.75-3.20 (m, 3H), 4.23-5.35 (m, 5H), 6.93-7.63 (m, 15H), 10.58-10.71 (m, 1H), chemical formula: C₃₂H₂₈F₃N₃O₅ m/z Calcd for [M + H]⁺ 592.20, m/z found 592.3. |
| 201-116 | 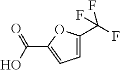 | 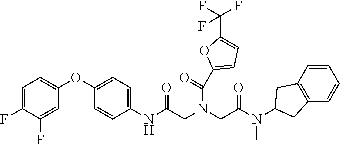 [SMILES: CN(C(=O)CN(CC(=O)Nc1ccc(Oc2ccc(F)c(F)c2)cc1)C(=O)c1ccc(C(F)(F)F)o1)C1Cc2ccccc2C1] | ¹H NMR (500 MHz, DMSO): δ 2.78-3.27 (m, 7H), 4.26-5.37 (m, 5H), 6.80-7.67 (m, 13H), 10.67-10.78 (m, 1H), chemical formula: C₃₂H₂₆F₅N₃O₅ m/z Calcd for [M + H]⁺ 628.18, m/z found 628.1. |
| 201-122 | 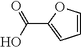 | 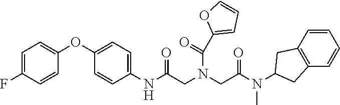 [SMILES: CN(C(=O)CN(CC(=O)Nc1ccc(Oc2ccc(F)cc2)cc1)C(=O)c1ccco1)C1Cc2ccccc2C1] | ¹H NMR (500 MHz, DMSO): δ 2.78-3.26 (m, 7H), 4.21 (s, 1H), 4.45-5.42 (m, 4H), 6.55-7.92 (m, 15H), 10.83-10.91 (m, 1H), chemical formula: C₃₁H₂₈FN₃O₅ m/z Calcd for [M + H]⁺ 542.20, m/z found 542.2. |
| 201-128 | 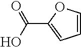 | 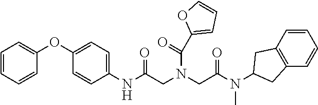 [SMILES: CN(C(=O)CN(CC(=O)Nc1ccc(Oc2ccccc2)cc1)C(=O)c1ccco1)C1Cc2ccccc2C1] | ¹H NMR (500 MHz, DMSO): δ 2.78-3.26 (m, 7H), 4.22 (s, 1H), 4.46-5.42 (m, 4H), 6.66-7.93 (m, 16H), 10.85-10.91 (m, 1H), chemical formula: C₃₁H₂₉N₃O₅ m/z Calcd for [M + H]⁺ 524.21, m/z found 524.6. |
| 201-134 | 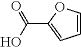 | 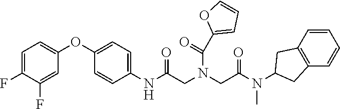 [SMILES: CN(C(=O)CN(CC(=O)Nc1ccc(Oc2ccc(F)c(F)c2)cc1)C(=O)c1ccco1)C1Cc2ccccc2C1] | ¹H NMR (500 MHz, DMSO): δ 2.78-3.26 (m, 7H), 4.22 (s, 1H), 4.45-5.42 (m, 4H), 6.55- 7.92 (m, 14H), 10.88-10.95 (m, 1H), chemical formula: C₃₁H₂₇F₂N₃O₅ m/z Calcd for [M + H]⁺ 560.19, m/z found 560.4. |
| 201-136 | 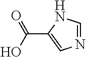 | 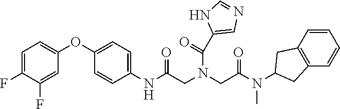 [SMILES: CN(C(=O)CN(CC(=O)Nc1ccc(Oc2ccc(F)c(F)c2)cc1)C(=O)c1cnc[nH]1)C1Cc2ccccc2C1] | ¹H NMR (500 MHz, DMSO): δ 2.74-3.31 (m, 7H), 4.07-5.36 (m, 5H), 6.76-7.77 (m, 13H), 10.89-11.22 (m, 1H), 12.55 (s, 1H), chemical formula: C₃₀H₂₇F₂N₅O₄ m/z Calcd for [M + H]⁺ 560.2, m/z found 560.4. |
| 201-137 | 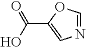 | 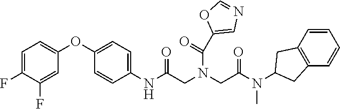 [SMILES: CN(C(=O)CN(CC(=O)Nc1ccc(Oc2ccc(F)c(F)c2)cc1)C(=O)c1cnco1)C1Cc2ccccc2C1] | ¹H NMR (500 MHz, DMSO): δ 2.48-3.30 (m, 7H), 4.22-5.33 (m, 5H), 6.77-7.68 (m, 12H), 8.57-8.61 (m, 1H), 10.64-10.80 (m, 1H), chemical formula: C₃₀H₂₆F₂N₄O₅ m/z Calcd for [M + H]⁺ 561.2, m/z found 561.3. |
| 201-138 | 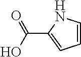 | 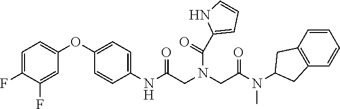 [SMILES: CN(C(=O)CN(CC(=O)Nc1ccc(Oc2ccc(F)c(F)c2)cc1)C(=O)c1ccc[nH]1)C1Cc2ccccc2C1] | ¹H NMR (500 MHz, DMSO): δ 2.80-3.31 (m, 7H), 4.19-5.38 (m, 5H), 6.16-7.66 (m, 14H), 10.90-11.53 (m, 1H), chemical formula: C₃₁H₂₈F₂N₄O₄ m/z Calcd for [M + H]⁺ 559.2, m/z found 559.4. |
| 201-139 | 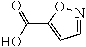 | 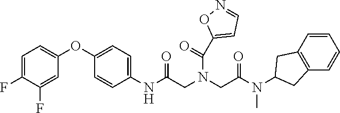 [SMILES: CN(C(=O)CN(CC(=O)Nc1ccc(Oc2ccc(F)c(F)c2)cc1)C(=O)c1ccno1)C1Cc2ccccc2C1] | ¹H NMR (500 MHz, DMSO): δ 2.49-3.31 (m, 7H), 4.27-5.32 (m, 5H), 6.77-7.65 (m, 12H), 8.74-8.76 (m, 1H), 10.59-11.63 (m, 1H), chemical formula: C₃₀H₂₆F₂N₄O₅ m/z Calcd for [M + H]⁺ 561.2, m/z found 561.3. |
| 201-141 | 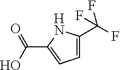 | 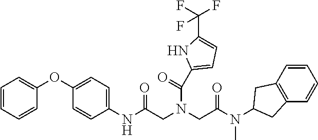 [SMILES: CN(C(=O)CN(CC(=O)Nc1ccc(Oc2ccccc2)cc1)C(=O)c1ccc(C(F)(F)F)[nH]1)C1Cc2ccccc2C1] | ¹H NMR (500 MHz, DMSO): δ 2.49-3.31 (m, 7H), 4.21 (s, 1H), 4.43-5.38 (m, 4H), 6.33- 7.63 (m, 15H), 10.66-10.99 (m, 1H), 12.84 (s, 1H), chemical formula: C₃₂H₂₉F₃N₄O₄ m/z Calcd for [M + H]⁺ 591.2, m/z found 591.4. |

### Preparation of compound 201-65

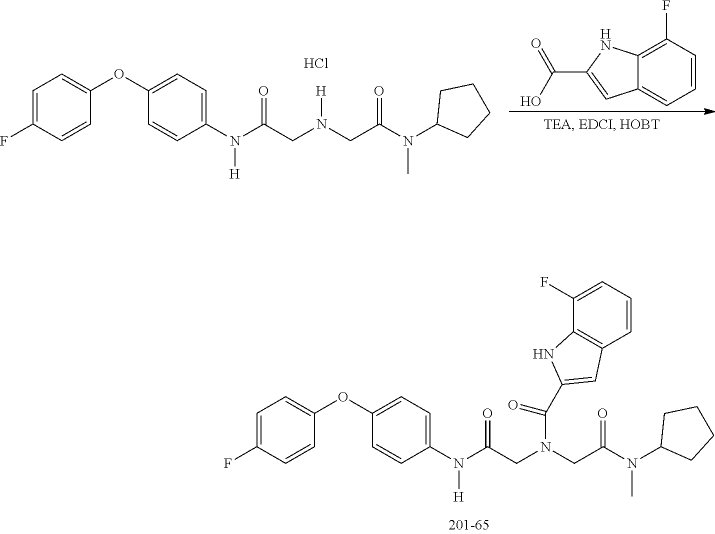 [SMILES: CN(C(=O)CNCC(=O)Nc1ccc(Oc2ccc(F)cc2)cc1)C1CCCC1 ; Cl ; O=C(O)c1cc2cccc(F)c2[nH]1 ; CN(C(=O)CN(CC(=O)Nc1ccc(Oc2ccc(F)cc2)cc1)C(=O)c1cc2cccc(F)c2[nH]1)C1CCCC1]

Compound 201-65 was prepared using the general procedure for the preparations of compounds of the present disclosure. Some other compounds were prepared according to the synthetic procedure of the compound 201-65 but using different carboxylic acids.

TABLE 2

| Com- | | | |
|---|---|---|---|
| pound | Carboxylic | | |
| No. | acid | Final compound | Identification data |
| 201-65 | 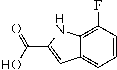 | 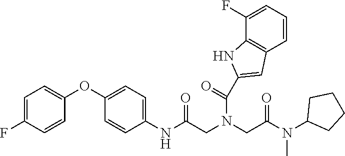 [SMILES: CN(C(=O)CN(CC(=O)Nc1ccc(Oc2ccc(F)cc2)cc1)C(=O)c1cc2cccc(F)c2[nH]1)C1CCCC1] | ¹H NMR (500 MHz, DMSO): δ 1.53-1.67 (m, 8H), 2.82-2.92 (m, 3H), 4.25 (s, 1H), 4.44-4.85 (m, 4H), 6.62-7.65 (m, 12H), 10.79-11.00 (m, 1H), 12.13 (s, 1H), chemical formula: C₃₁H₃₀F₂N₄O₄ m/z Calcd for [M + H]⁺ 561.2, m/z found 561.2. |
| 201-70 | 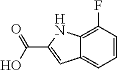 | 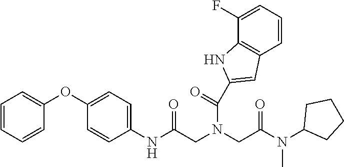 [SMILES: CN(C(=O)CN(CC(=O)Nc1ccc(Oc2ccccc2)cc1)C(=O)c1cc2cccc(F)c2[nH]1)C1CCCC1] | ¹H NMR (500 MHz, DMSO): δ 1.53-1.67 (m, 8H), 2.80-2.92 (m, 3H), 4.25 (s, 1H), 4.44-4.86 (m, 4H), 6.62-7.66 (m, 13H), 10.80-11.00 (m, 1H), 12.14 (s, 1H), chemical formula: C₃₁H₃₁FN₄O₄ m/z Calcd for [M + H]⁺ 543.24, m/z found 543.3. |
| 201-75 | 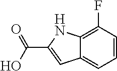 | 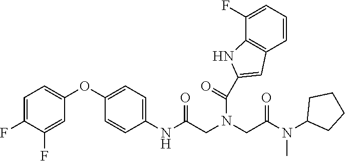 [SMILES: CN(C(=O)CN(CC(=O)Nc1ccc(Oc2ccc(F)c(F)c2)cc1)C(=O)c1cc2cccc(F)c2[nH]1)C1CCCC1] | ¹H NMR (500 MHz, DMSO): δ 1.54-1.68 (m, 8H), 2.83-2.92 (m, 3H), 4.25 (s, 1H), 4.44-4.86 (m, 4H), 6.62-7.67 (m, 11H), 10.84-11.03 (m, 1H), 12.13 (s, 1H), chemical formula: C₃₁H₂₉F₃N₄O₄ m/z Calcd for [M + H]⁺ 579.22, m/z found 579.2. |
| 201-81 | 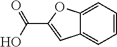 | 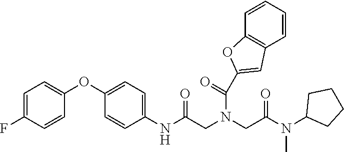 [SMILES: CN(C(=O)CN(CC(=O)Nc1ccc(Oc2ccc(F)cc2)cc1)C(=O)c1cc2ccccc2o1)C1CCCC1] | ¹H NMR (500 MHz, DMSO): δ 1.54-1.68 (m, 8H), 2.79-2.94 (m, 3H), 4.25-4.94 (m, 5H), 6.97-7.78 (m, 13H), 10.78-10.84 (m, 1H), 12.14 (s, 1H), chemical formula: C₃₁H₃₀FN₃O₅ m/z Calcd for [M + H]⁺ 544.22, m/z found 545.3. |
| 201-93 | 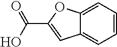 | 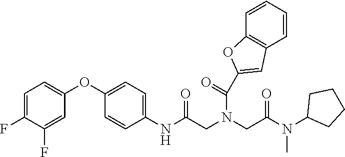 [SMILES: CN(C(=O)CN(CC(=O)Nc1ccc(Oc2ccc(F)c(F)c2)cc1)C(=O)c1cc2ccccc2o1)C1CCCC1] | ¹H NMR (500 MHz, DMSO): δ 1.54-1.86 (m, 8H), 2.79-2.94 (m, 3H), 4.26-4.95 (m, 5H), 6.77-7.78 (m, 12H), 10.82-10.89 (m, 1H), chemical formula: C₃₁H₂₉F₂N₃O₅ m/z Calcd for [M + H]⁺ 562.21, m/z found 562.0. |
| 201-111 | 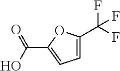 | 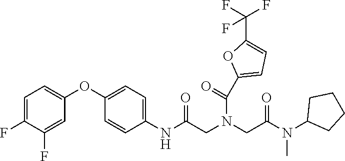 [SMILES: CN(C(=O)CN(CC(=O)Nc1ccc(Oc2ccc(F)c(F)c2)cc1)C(=O)c1ccc(C(F)(F)F)o1)C1CCCC1] | ¹H NMR (500 MHz, DMSO): δ 1.53-1.86 (m 8H), 2.79-2.89 (m, 3H), 4.14-4.88 (m, 5H), 6.80-7.66 (m, 9H), 10.72-10.82 (m, 1H), chemical formula: C₂₈H₂₆F₅N₃O₅ m/z Calcd for [M + H]⁺ 580.18, m/z found 580.2. |
| 201-117 | 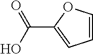 | 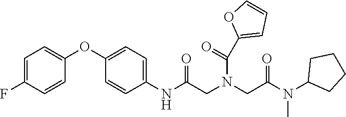 [SMILES: CN(C(=O)CN(CC(=O)Nc1ccc(Oc2ccc(F)cc2)cc1)C(=O)c1ccco1)C1CCCC1] | ¹H NMR (500 MHz, DMSO): δ 1.54-1.89 (m, 8H), 2.79-2.90 (m, 3H), 4.20-4.86 (m, 5H), 6.64-7.88 (m, 11H), 10.95-11.07 (m, 1H), chemical formula: C₂₇H₂₈FN₃O₅ m/z Calcd for [M + H]⁺ 494.20, m/z found 494.1. |
| 201-123 | 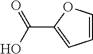 | 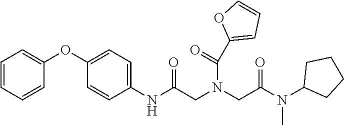 [SMILES: CN(C(=O)CN(CC(=O)Nc1ccc(Oc2ccccc2)cc1)C(=O)c1ccco1)C1CCCC1] | ¹H NMR (500 MHz, DMSO): δ 1.25-1.89 (m, 8H), 2.79-2.90 (m, 3H), 4.20-4.86 (m, 5H), 6.64-7.88 (m, 12H), 10.89-10.97 (m, 1H), chemical formula: C₂₇H₂₈N₃O₅ m/z Calcd for [M + H]⁺ 476.21, m/z found 476.1. |
| 201-129 | 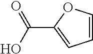 | 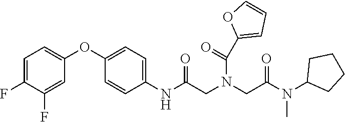 [SMILES: CN(C(=O)CN(CC(=O)Nc1ccc(Oc2ccc(F)c(F)c2)cc1)C(=O)c1ccco1)C1CCCC1] | ¹H NMR (500 MHz, DMSO): δ 1.56-1.90 (m, 8H), 2.80-2.91 (m, 3H), 4.20-4.87 (m, 5H), 6.66-7.88 (m, 10H), 10.94-11.01 (m, 1H), chemical formula: C₂₇H₂₇F₂N₃O₅ m/z Calcd for [M + H]⁺ 512.19, m/z found 512.2. |
| 201-143 | 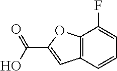 | 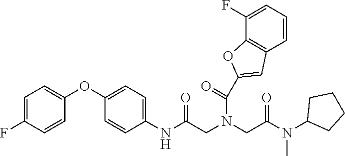 [SMILES: CN(C(=O)CN(CC(=O)Nc1ccc(Oc2ccc(F)cc2)cc1)C(=O)c1cc2cccc(F)c2o1)C1CCCC1] | ¹H NMR (500 MHz, DMSO): δ 1.52-1.84 (m, 8H), 2.79-2.92 (2s, 3H), 4.17-4.92 (m, 5H), 6.97-7.63 (m, 10H), 10.68-10.78 (m, 1H), chemical formula: C₃₁H₂₉F₂N₃O₅ m/z Calcd for [M + H]⁺ 562.2, m/z found 562.3. |
| 201-227 | 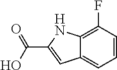 | 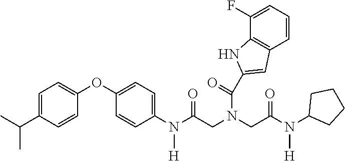 [SMILES: CC(C)c1ccc(Oc2ccc(NC(=O)CN(CC(=O)NC3CCCC3)C(=O)c3cc4cccc(F)c4[nH]3)cc2)cc1] | ¹H NMR (500 MHz, DMSO): δ 1.18-1.20 (m, 6H), 1.36-1.84 (m, 8H), 2.83-2.92 (m, 1H), 4.06-4.55 (m, 5H), 6.81-7.64 (m, 12H), 8.33-8.63 (m, 1H), 10.64-11.08 (2s, 1H), 12.16 (s, 1H), chemical formula: C₃₃H₃₅FN₄O₄ m/z Calcd for [M + H]⁺ 571.26, m/z found 571.1. |
| 201-235 | 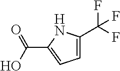 | 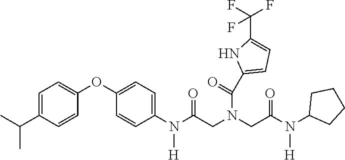 [SMILES: CC(C)c1ccc(Oc2ccc(NC(=O)CN(CC(=O)NC3CCCC3)C(=O)c3ccc(C(F)(F)F)[nH]3)cc2)cc1] | ¹H NMR (500 MHz, DMSO): δ 1.18-1.20 (m, 6H), 1.36-1.81 (m, 8H), 2.83-2.92 (m, 1H), 4.04-4.47 (m, 5H), 6.44-7.62 (m, 10H), 8.32-8.66 (m, 1H), 10.58-11.14 (2s, 1H), 12.86 (s, 1H), chemical formula: C₃₀H₃₃F₃N₄O₄ m/z Calcd for [M + H]⁺ 571.25, m/z found 571.1. |
| 201-237 | 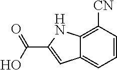 | 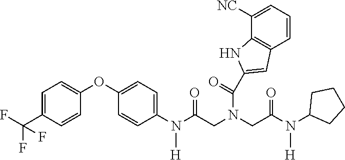 [SMILES: N#Cc1cccc2cc(C(=O)N(CC(=O)Nc3ccc(Oc4ccc(C(F)(F)F)cc4)cc3)CC(=O)NC3CCCC3)[nH]c12] | ¹H NMR (500 MHz, DMSO): δ 1.18-1.88 (m, 8H), 4.04-4.55 (m, 5H), 6.90-8.56 (m, 13H), 10.78-11.13 (2s, 1H), 12.59-12.64 (m, 1H), chemical formula: C₃₂H₂₈F₃N₅O₄ m/z Calcd for [M + H]⁺ 604.21, m/z found 604.1. |

### Preparation of Compound 201-74

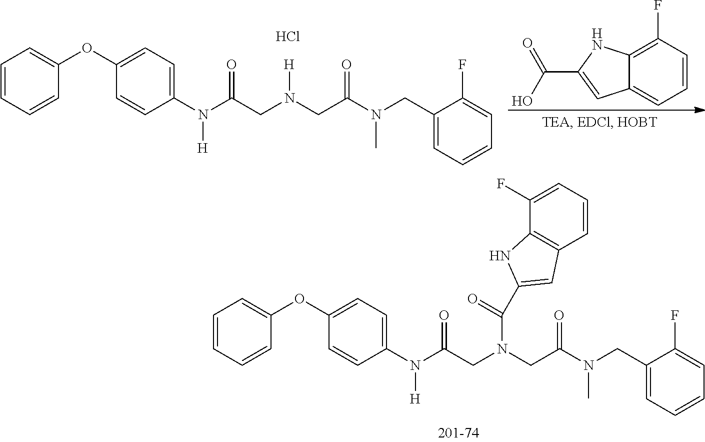

Compound 201-74 was prepared using the general procedure for the preparations of compounds of the present disclosure. Some other compounds were prepared according to the synthetic procedure of the compound 201-74 but using different carboxylic acids and diamide intermediates.

TABLE 3

| Com- | | | |
|---|---|---|---|
| pound | Carboxylic | | |
| No. | acid | Final compound | Identification data |
| 201-37 | 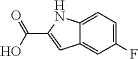 | 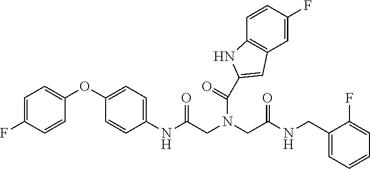 [SMILES: O=C(CN(CC(=O)Nc1ccc(Oc2ccc(F)cc2)cc1)C(=O)c1cc2cc(F)ccc2[nH]1)NCc1ccccc1F] | ¹H NMR (500 MHz, DMSO): δ 4.32-4.62 (m, 6H), 6.69-7.67 (m, 16H), 8.93-9.20 (m, 1H), 10.61-11.05 (2s, 1H), 11.78 (bs, 1H), chemical formula: C₃₂H₂₅F₃N₄O₄ m/z Calcd for [M + H]⁺ 587.1, m/z found 587.5. |
| 201-41 | 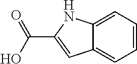 | 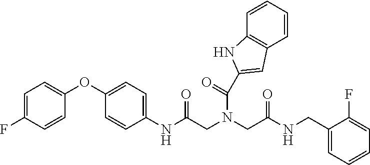 [SMILES: O=C(CN(CC(=O)Nc1ccc(Oc2ccc(F)cc2)cc1)C(=O)c1cc2ccccc2[nH]1)NCc1ccccc1F] | ¹H NMR (500 MHz, DMSO): δ 4.32 (s, 2H), 4.44 (s, 2H), 4.57-4.62 (m, 2H), 6.70-7.63 (m, 17H), 8.90-9.15 (m, 1H), 10.54, 11.03 (2s, 1H), 11.66 (bs, 1H), chemical formula: C₃₂H₂₆F₂N₄O₄ m/z Calcd for [M + H]⁺ 569.1, m/z found 569.5. |
| 201-43 | 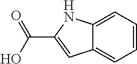 | 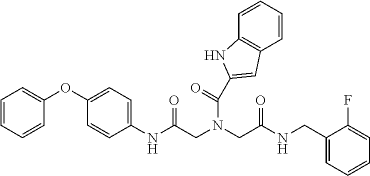 [SMILES: O=C(CN(CC(=O)Nc1ccc(Oc2ccccc2)cc1)C(=O)c1cc2ccccc2[nH]1)NCc1ccccc1F] | ¹H NMR (500 MHz, DMSO): δ 4.33 (s, 2H), 4.44 (s, 2H), 4.58-4.63 (m, 2H), 6.70-7.64 (m, 18H), 8.90-9.16 (m, 1H), 10.55, 11.03 (2s, 1H), 11.66 (s, 1H), chemical formula: C₃₂H₂₇FN₄O₄ m/z Calcd for [M + H]⁺ 551.2, m/z found 551.4. |
| 201-45 | 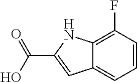 | 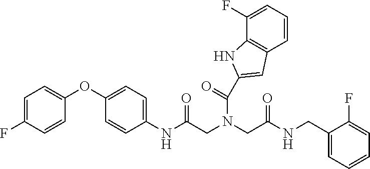 [SMILES: O=C(CN(CC(=O)Nc1ccc(Oc2ccc(F)cc2)cc1)C(=O)c1cc2cccc(F)c2[nH]1)NCc1ccccc1F] | ¹H NMR (500 MHz, DMSO): δ 4.30-4.61 (m, 6H), 6.78-7.67 (m, 16H), 9.00-9.26 (m, 1H), 10.48, 10.48, 11.08 (2s, 1H), 12.17 (s, 1H), chemical formula: C₃₂H₂₅F₃N₄O₄ m/z Calcd for [M + H]⁺ 587.1, m/z found 587.3. |
| 201-46 | 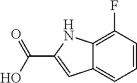 | 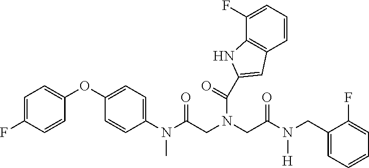 [SMILES: CN(C(=O)CN(CC(=O)NCc1ccccc1F)C(=O)c1cc2cccc(F)c2[nH]1)c1ccc(Oc2ccc(F)cc2)cc1] | ¹H NMR (500 MHz, DMSO): δ 3.19-3.21 (m, 3H), 4.03-4.41 (m, 6H), 6.63 (s, 1H), 6.93-7.54 (m, 15H), 8.67-9.16 (m, 1H), 12.07-12.13 (m, 1H), chemical formula: C₃₃H₂₇F₃N₄O₄ m/z Calcd for [M + H]⁺ 601.20, m/z found 601.34. |
| 201-47 | 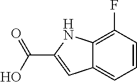 | 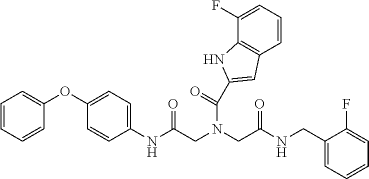 [SMILES: O=C(CN(CC(=O)Nc1ccc(Oc2ccccc2)cc1)C(=O)c1cc2cccc(F)c2[nH]1)NCc1ccccc1F] | ¹H NMR (500 MHz, DMSO): δ 4.30-4.60 (m, 6H), 6.77-7.67 (m, 17H), 8.92-9.16 (m, 1H), 10.62, 10.98 (2s, 1H), 12.17 (s, 1H), chemical formula: C₃₂H₂₆F₂N₄O₄ m/z Calcd for [M + H]⁺ 569.1, m/z found 569.3. |
| 201-49 | 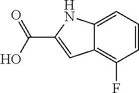 | 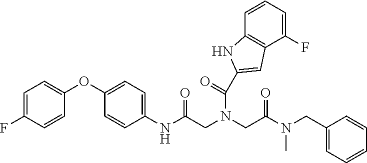 [SMILES: CN(Cc1ccccc1)C(=O)CN(CC(=O)Nc1ccc(Oc2ccc(F)cc2)cc1)C(=O)c1cc2c(F)cccc2[nH]1] | ¹H NMR (500 MHz, DMSO): δ 2.88-3.32 (m, 3H), 4.25-4.97 (m, 6H), 6.81-7.64 (m, 17H), 10.59-11.02 (m, 1H), 12.02-12.07 (m, 1H), chemical formula: C₃₃H₂₈F₂N₄O₄ m/z Calcd for [M + H]⁺ 583.2, m/z found 583.4. |
| 201-50 | 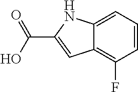 | 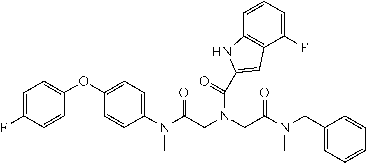 [SMILES: CN(Cc1ccccc1)C(=O)CN(CC(=O)N(C)c1ccc(Oc2ccc(F)cc2)cc1)C(=O)c1cc2c(F)cccc2[nH]1] | ¹H NMR (500 MHz, DMSO): δ 2.66-3.29 (m, 6H), 4.00-4.73 (m, 6H), 6.49-7.43 (m, 17H), 11.94-12.00 (m, 1H), chemical formula: C₃₄H₃₀F₂N₄O₄ m/z Calcd for [M + H]⁺ 597.2, m/z found 597.5. |
| 201-51 | 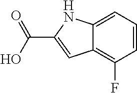 | 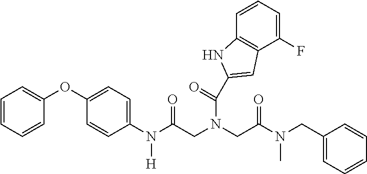 [SMILES: CN(Cc1ccccc1)C(=O)CN(CC(=O)Nc1ccc(Oc2ccccc2)cc1)C(=O)c1cc2c(F)cccc2[nH]1] | ¹H NMR (500 MHz, DMSO): δ 2.88-3.32 (m, 3H), 4.26-4.79 (m, 6H), 6.81-7.68 (m, 18H), 10.60-11.03 (m, 1H), 12.02-12.07 (m, 1H), chemical formula: C₃₃H₂₉FN₄O₄ m/z Calcd for [M + H]⁺ 565.22, m/z found 565.5. |
| 201-52 | 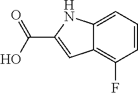 | 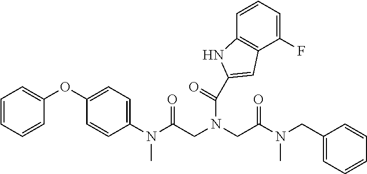 [SMILES: CN(Cc1ccccc1)C(=O)CN(CC(=O)N(C)c1ccc(Oc2ccccc2)cc1)C(=O)c1cc2c(F)cccc2[nH]1] | ¹H NMR (500 MHz, DMSO): δ 2.49-3.32 (m, 6H), 4.00-4.73 (m, 6H), 6.49-7.45 (m, 18H), 11.95-12.00 (m, 1H), chemical formula: C₃₄H₃₁FN₄O₄ m/z Calcd for [M + H]⁺ 579.24, m/z found 579.5. |
| 201-53 | 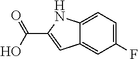 | 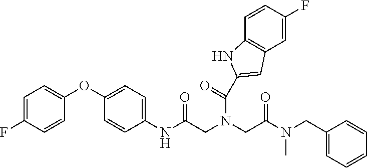 [SMILES: CN(Cc1ccccc1)C(=O)CN(CC(=O)Nc1ccc(Oc2ccc(F)cc2)cc1)C(=O)c1cc2cc(F)ccc2[nH]1] | ¹H NMR (500 MHz, DMSO): δ 2.88-3.32 (m, 3H), 4.24-4.91 (m, 6H), 6.64-7.68 (m, 17 H), 10.60-11.00 (m, 1H), 11.74-11.83 (m, 1H), chemical formula: C₃₃H₂₈F₂N₄O₄ m/z Calcd for [M + H]⁺ 583.2, m/z found 583.5. |
| 201-54 | 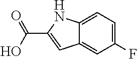 | 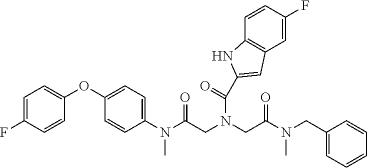 [SMILES: CN(Cc1ccccc1)C(=O)CN(CC(=O)N(C)c1ccc(Oc2ccc(F)cc2)cc1)C(=O)c1cc2cc(F)ccc2[nH]1] | ¹H NMR (500 MHz, DMSO): δ 2.49-3.32 (m, 6H), 3.94-4.68 (m, 6H), 6.36-6.56 (m, 1H), 6.97-7.53 (m, 16H), 11.68-11.74 (m, 1H), chemical formula: C₃₄H₃₀F₂N₄O₄ m/z Calcd for [M + H]⁺ 597.23, m/z found 597.51. |
| 201-55 | 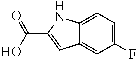 | 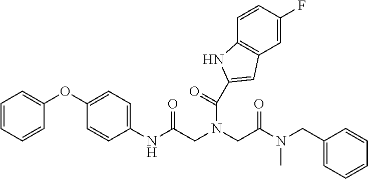 [SMILES: CN(Cc1ccccc1)C(=O)CN(CC(=O)Nc1ccc(Oc2ccccc2)cc1)C(=O)c1cc2cc(F)ccc2[nH]1] | ¹H NMR (500 MHz, DMSO): δ 2.88-3.32 (m, 3H), 4.24-4.91 (m, 6H), 6.64-7.69 (m, 18H), 10.61-11.00 (m, 1H), 11.74-11.83 (m, 1H), chemical formula: C₃₃H₂₉FN₄O₄ m/z Calcd for [M + H]⁺ 565.2, m/z found 565.4. |
| 201-57 | 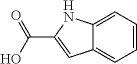 | 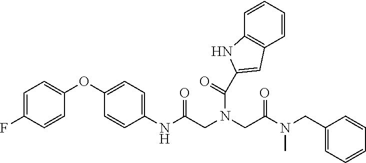 [SMILES: CN(Cc1ccccc1)C(=O)CN(CC(=O)Nc1ccc(Oc2ccc(F)cc2)cc1)C(=O)c1cc2ccccc2[nH]1] | ¹H NMR (500 MHz, DMSO): δ 2.88-3.32 (m, 3H), 4.24-4.93 (m, 6H), 6.50-7.69 (m, 18H), 10.70-11.06 (m, 1H), 11.66-11.71 (m, 1H), chemical formula: C₃₃H₂₉FN₄O₄ m/z Calcd for [M + H]⁺ 565.2, m/z found 565.5. |
| 201-69 | 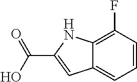 | 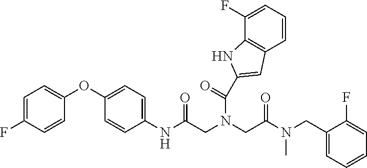 [SMILES: CN(Cc1ccccc1F)C(=O)CN(CC(=O)Nc1ccc(Oc2ccc(F)cc2)cc1)C(=O)c1cc2cccc(F)c2[nH]1] | ¹H NMR (500 MHz, DMSO): δ 2.89-3.09 (m, 3H), 4.24-4.89 (m, 6H), 6.61-7.68 (m, 16H), 10.57-10.83 (m, 1H), 12.16-12.25 (m, 1H), chemical formula: C₃₃H₂₇F₃N₄O₄ m/z Calcd for [M + H]⁺ 601.2, m/z found 601.3. |
| 201-74 | 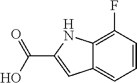 | 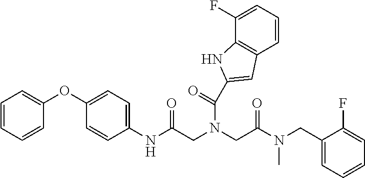 [SMILES: CN(Cc1ccccc1F)C(=O)CN(CC(=O)Nc1ccc(Oc2ccccc2)cc1)C(=O)c1cc2cccc(F)c2[nH]1] | ¹H NMR (500 MHz, DMSO): δ 2.90-3.09 (m, 3H), 4.24-4.88 (m, 6H), 6.62-7.65 (m, 17H), 10.53-10.80 (m, 1H), 12.10-12.19 (m, 1H), chemical formula: C₃₃H₂₈F₂N₄O₄ m/z Calcd for [M + H]⁺ 583.21, m/z found 583.1. |
| 201-77 | 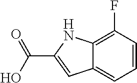 | 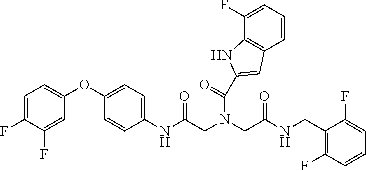 [SMILES: O=C(CN(CC(=O)Nc1ccc(Oc2ccc(F)c(F)c2)cc1)C(=O)c1cc2cccc(F)c2[nH]1)NCc1c(F)cccc1F] | ¹H NMR (500 MHz, DMSO): δ 4.22-4.58 (m, 6H), 6.65-7.68 (m, 14H), 8.84-9.15 (m, 1H), 10.76-11.12 (m, 1H), 12.13 (s, 1H), chemical formula: C₃₂H₂₃F₅N₄O₄ m/z Calcd for [M + H]⁺ 623.17, m/z found 623.1. |
| 201-85 | 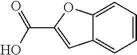 | 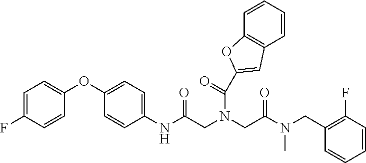 [SMILES: CN(Cc1ccccc1F)C(=O)CN(CC(=O)Nc1ccc(Oc2ccc(F)cc2)cc1)C(=O)c1cc2ccccc2o1] | ¹H NMR (500 MHz, DMSO): δ 2.89-3.07 (m, 3H), 4.26-4.97 (m, 6H), 6.90-7.79 (m, 17H), 10.53-10.67 (m, 1H), chemical formula: C₃₃H₂₇F₂N₃O₅ m/z Calcd for [M + H]⁺ 584.19, m/z found 584.3. |
| 201-91 | 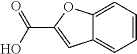 | 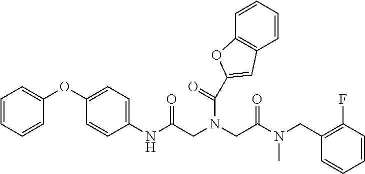 [SMILES: CN(Cc1ccccc1F)C(=O)CN(CC(=O)Nc1ccc(Oc2ccccc2)cc1)C(=O)c1cc2ccccc2o1] | ¹H NMR (500 MHz, DMSO): δ 2.89-3.08 (m, 3H), 4.27-4.97 (m, 6H), 6.90-7.79 (m, 18H), 10.54-10.66 (m, 1H), chemical formula: C₃₃H₂₈FN₃O₅ m/z Calcd for [M + H]⁺ 566.20, m/z found 566.3. |
| 201-97 | 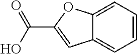 | 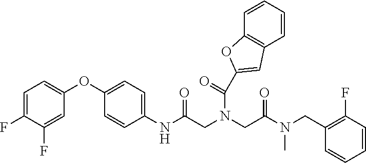 [SMILES: CN(Cc1ccccc1F)C(=O)CN(CC(=O)Nc1ccc(Oc2ccc(F)c(F)c2)cc1)C(=O)c1cc2ccccc2o1] | ¹H NMR (500 MHz, DMSO): δ 2.90-3.10 (m, 3H), 4.28-4.99 (m, 6H), 6.80-7.80 (m, 16H), 10.62-10.72 (m, 1H), chemical formula: C₃₃H₂₆F₃N₃O₅ m/z Calcd for [M + H]⁺ 602.18, m/z found 602.4. |
| 201-103 | 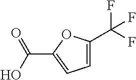 | 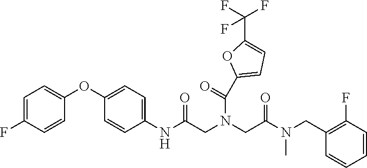 [SMILES: CN(Cc1ccccc1F)C(=O)CN(CC(=O)Nc1ccc(Oc2ccc(F)cc2)cc1)C(=O)c1ccc(C(F)(F)F)o1] | ¹H NMR (500 MHz, DMSO): δ 2.91-3.09 (m, 3H), 4.28-4.91 (m, 6H), 7.01-7.64 (m, 14H), 10.51-10.67 (m, 1H), chemical formula: C₃₀H₂₄F₅N₃O₅ m/z Calcd for [M + H]⁺ 602.16, m/z found 602.3. |
| 201-109 | 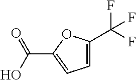 | 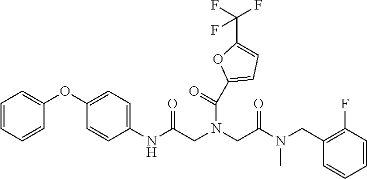 [SMILES: CN(Cc1ccccc1F)C(=O)CN(CC(=O)Nc1ccc(Oc2ccccc2)cc1)C(=O)c1ccc(C(F)(F)F)o1] | ¹H NMR (500 MHz, DMSO): δ 2.89-3.07 (m, 3H), 4.26-4.89 (m, 6H), 6.97-7.64 (m, 15H), 10.49-10.65 (m, 1H), chemical formula: C₃₀H₂₅F₄N₃O₅ m/z Calcd for [M + H]⁺ 584.17, m/z found 584.3. |
| 201-115 | 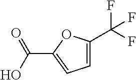 | 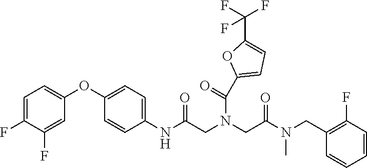 [SMILES: CN(Cc1ccccc1F)C(=O)CN(CC(=O)Nc1ccc(Oc2ccc(F)c(F)c2)cc1)C(=O)c1ccc(C(F)(F)F)o1] | ¹H NMR (500 MHz, DMSO): δ 2.88-3.06 (m, 3H), 4.25-4.88 (m, 6H), 6.80-7.65 (m, 13H), 10.52-10.68 (m, 1H), chemical formula: C₃₀H₂₃F₆N₃O₅ m/z Calcd for [M + H]⁺ 620.15, m/z found 620.2. |
| 201-127 | 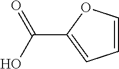 | 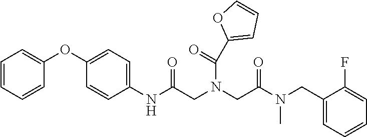 [SMILES: CN(Cc1ccccc1F)C(=O)CN(CC(=O)Nc1ccc(Oc2ccccc2)cc1)C(=O)c1ccco1] | ¹H NMR (500 MHz, DMSO): δ 2.88-3.05 (m, 3H), 4.18-4.86 (m, 6H), 6.61-7.86 (m, 16H), 10.54-10.76 (m, 1H), chemical formula: C₂₉H₂₆FN₃O₅ m/z Calcd for [M + H]⁺ 516.19, m/z found 516.2. |
| 201-131 | 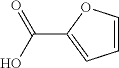 | 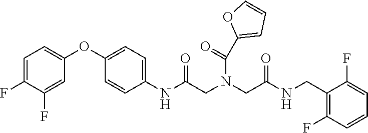 [SMILES: O=C(CN(CC(=O)Nc1ccc(Oc2ccc(F)c(F)c2)cc1)C(=O)c1ccco1)NCc1c(F)cccc1F] | ¹H NMR (500 MHz, DMSO): δ 4.19-4.50 (m, 6H), 6.61-7.81 (m, 13H), 8.82-9.05 (m, 1H), 10.62, 10.93 (2s, 1H), chemical formula: C₂₈H₂₁F₄N₃O₅ m/z Calcd for [M + H]⁺ 556.14, m/z found 556.3. |
| 201-133 | 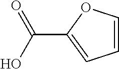 | 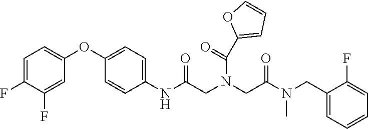 [SMILES: CN(Cc1ccccc1F)C(=O)CN(CC(=O)Nc1ccc(Oc2ccc(F)c(F)c2)cc1)C(=O)c1ccco1] | ¹H NMR (500 MHz, DMSO): δ 2.75-3.07 (m, 3H), 4.19-4.88 (m, 6H), 6.64-7.88 (m, 14H), 10.61-10.83 (m, 1H), chemical formula: C₂₉H₂₄F₃N₃O₅ m/z Calcd for [M + H]⁺ 552.17, m/z found 552.3. |
| 201-145 | 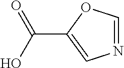 | 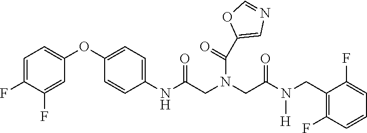 [SMILES: O=C(CN(CC(=O)Nc1ccc(Oc2ccc(F)c(F)c2)cc1)C(=O)c1cnco1)NCc1c(F)cccc1F] | ¹H NMR (500 MHz, DMSO): δ 4.18-4.48 (m, 6H), 6.79-7.67 (m, 11H), 8.47, 8.57 (2s, 1H), 8.74-8.98 (m, 1H), 10.47, 10.78 (2s, 1H), chemical formula: C₂₇H₂₀F₄N₄O₅ m/z Calcd for [M + H]⁺ 557.1, m/z found 557.3. |
| 201-205 | 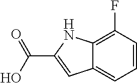 | 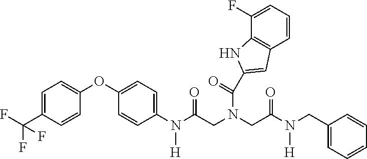 [SMILES: O=C(CN(CC(=O)Nc1ccc(Oc2ccc(C(F)(F)F)cc2)cc1)C(=O)c1cc2cccc(F)c2[nH]1)NCc1ccccc1] | ¹H NMR (500 MHz, DMSO): δ 4.33-4.61 (m, 6H), 6.80-7.73 (m, 17H), 8.88-9.08 (m, 1H), 10.66-11.06 (2s, 1H), 12.18 (s, 1H), chemical formula: C₃₃H₂₆F₄N₄O₄ m/z Calcd for [M + H]⁺ 619.2, m/z found 619.0. |
| 201-209 | 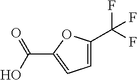 | 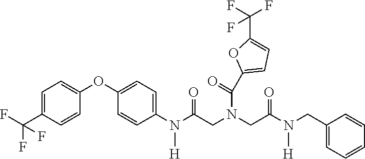 [SMILES: O=C(CN(CC(=O)Nc1ccc(Oc2ccc(C(F)(F)F)cc2)cc1)C(=O)c1ccc(C(F)(F)F)o1)NCc1ccccc1] | ¹H NMR (500 MHz, DMSO): δ 4.27-4.53 (m, 6H), 7.08-7.73 (m, 15H), 8.81-8.99 (m, 1H), 10.55-10.79 (2s, 1H), chemical formula: C₃₀H₂₃F₆N₃O₅ m/z Calcd for [M + H]⁺ 620.15, m/z found 620.0. |
| 201-242 | 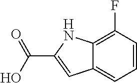 | 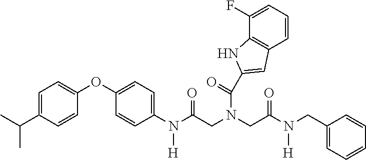 [SMILES: CC(C)c1ccc(Oc2ccc(NC(=O)CN(CC(=O)NCc3ccccc3)C(=O)c3cc4cccc(F)c4[nH]3)cc2)cc1] | ¹H NMR (500 MHz, DMSO): δ 1.18-1.24 (m, 6H), 2.83-2.92 (m, 1H), 4.31-4.58 (m, 6H), 6.79-7.63 (m, 17H), 8.87-9.08 (m, 1H), 10.55-10.94 (2s, 1H), 12.17 (s, 1H), chemical formula: C₃₅H₃₃FN₄O₄ m/z Calcd for [M + H]⁺ 593.25, m/z found 593.0. |
| 201-245 | 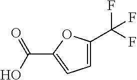 | 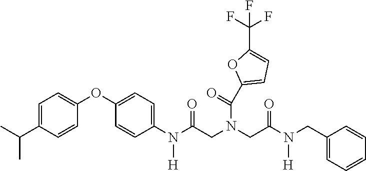 [SMILES: CC(C)c1ccc(Oc2ccc(NC(=O)CN(CC(=O)NCc3ccccc3)C(=O)c3ccc(C(F)(F)F)o3)cc2)cc1] | ¹H NMR (500 MHz, DMSO): δ 1.18-1.20 (m, 6H), 2.83-2.92 (m, 1H), 4.25-4.51 (m, 6H), 6.87-7.59 (m, 15H), 8.80-9.00 (m, 1H), 10.43-10.68 (2s, 1H), chemical formula: C₃₂H₃₀F₃N₃O₅ m/z Calcd for [M + H]⁺ 594.21, m/z found 594.0. |
| 201-249 | 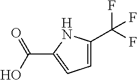 | 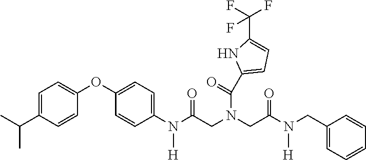 [SMILES: CC(C)c1ccc(Oc2ccc(NC(=O)CN(CC(=O)NCc3ccccc3)C(=O)c3ccc(C(F)(F)F)[nH]3)cc2)cc1] | ¹H NMR (500 MHz, DMSO): δ 1.17-1.19 (m, 6H), 2.82-2.91 (m, 1H), 4.25-4.50 (m, 6H), 6.42-7.59 (m, 15H), 8.84-9.09 (m, 1H), 10.47-10.98 (2s, 1H), 12.86 (s, 1H), chemical formula: C₃₂H₃₁FN₄O₄ m/z Calcd for [M + H]⁺ 593.23, m/z found 593.1. |
| 201-253 | 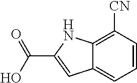 | 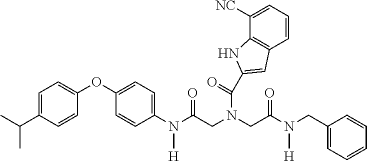 [SMILES: CC(C)c1ccc(Oc2ccc(NC(=O)CN(CC(=O)NCc3ccccc3)C(=O)c3cc4cccc(C#N)c4[nH]3)cc2)cc1] | ¹H NMR (500 MHz, DMSO): δ 1.17-1.20 (m, 6H), 2.82-2.91 (m, 1H), 4.30-4.55 (m, 6H), 6.86-8.02 (m, 17H), 8.85-9.03 (m, 1H), 10.53-10.87 (2s, 1H), 12.60-12.63 (m, 1H), chemical formula: C₃₆H₃₃N₅O₄ m/z Calcd for [M + H]⁺ 600.25, m/z found 600.1. |

### Preparation of Compound 201-5

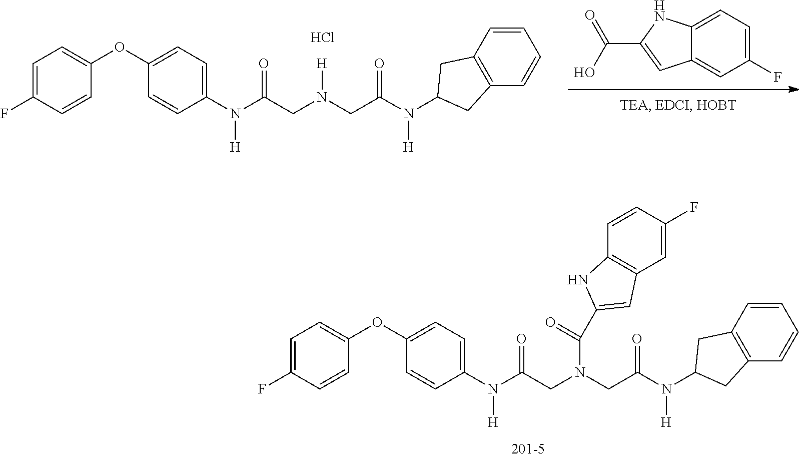 [SMILES: O=C(CNCC(=O)NC1Cc2ccccc2C1)Nc1ccc(Oc2ccc(F)cc2)cc1 ; Cl ; O=C(O)c1cc2cc(F)ccc2[nH]1 ; O=C(CN(CC(=O)NC1Cc2ccccc2C1)C(=O)c1cc2cc(F)ccc2[nH]1)Nc1ccc(Oc2ccc(F)cc2)cc1]

Compound 201-5 was prepared using the general procedure for the preparations of compounds of the present disclosure. Some other compounds were prepared according to the synthetic procedure of the compound 201-5 but using different carboxylic acids and diamide intermediates.

TABLE 4

| Com- | | | |
|---|---|---|---|
| pound | Carboxylic | | |
| No. | acid | Final compound | Identification data |
| 201-2 | 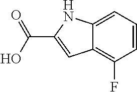 | 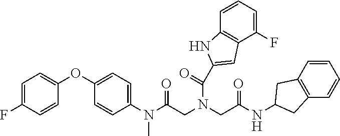 [SMILES: CN(C(=O)CN(CC(=O)NC1Cc2ccccc2C1)C(=O)c1cc2c(F)cccc2[nH]1)c1ccc(Oc2ccc(F)cc2)cc1] | ¹H NMR (500 MHz, DMSO): δ 2.73-3.33 (m, 7H), 4.03-4.58 (m, 5H), 6.56-7.46 (m, 16H), 8.47-9.07 (m, 1H), 11.98 (s, 1H), chemical formula: C₃₅H₃₀F₂N₄O₄ m/z Calcd for [M+ H]⁺ 609.2, m/z found 609.5. |
| 201-4 | 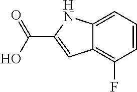 | 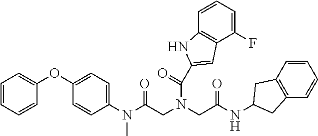 [SMILES: CN(C(=O)CN(CC(=O)NC1Cc2ccccc2C1)C(=O)c1cc2c(F)cccc2[nH]1)c1ccc(Oc2ccccc2)cc1] | ¹H NMR (500 MHz, DMSO): δ 2.69-3.32 (m, 7H), 4.02-4.54 (m, 5H), 6.57-7.46 (m, 17 H), 8.45-9.04 (m, 1H), 11.98 (s, 1H), chemical formula: C₃₅H₃₁FN₄O₄ m/z Calcd for [M+ H]⁺ 591.2, m/z found 591.5. |
| 201-5 | 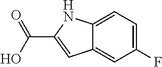 | 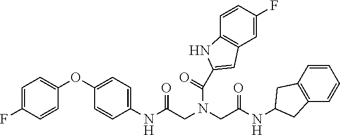 [SMILES: O=C(CN(CC(=O)NC1Cc2ccccc2C1)C(=O)c1cc2cc(F)ccc2[nH]1)Nc1ccc(Oc2ccc(F)cc2)cc1] | ¹H NMR (500 MHz, DMSO): δ 2.69-3.32 (m, 4H), 4.22-4.58 (m, 5H), 6.71 (s, 1H), 6.99-7.68 (m, 15H), 8.72-8.96 (m, 1H), 10.62, 11.14 (2s, 1H), 11.77 (s, 1H), chemical formula: C₃₄H₂₈F₂N₄O₄ m/z Calcd for [M+ H]⁺ 595.2, m/z found 595.4. |
| 201-8 | 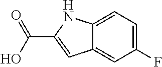 | 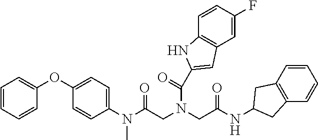 [SMILES: CN(C(=O)CN(CC(=O)NC1Cc2ccccc2C1)C(=O)c1cc2cc(F)ccc2[nH]1)c1ccc(Oc2ccccc2)cc1] | ¹H NMR (500 MHz, DMSO): δ 2.73-3.32 (m, 7H), 4.02-4.60 (m, 5H), 6.51-7.53 (m, 17H), 8.44-9.04 (m, 1H), 11.70, 11.75 (m, 1H), chemical formula: C₃₅H₃₁FN₄O₄ m/z Calcd for [M+ H]⁺ 591.2, m/z found 591.5. |
| 201-9 | 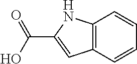 | 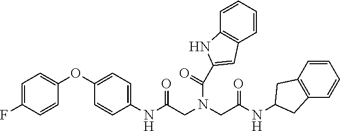 [SMILES: O=C(CN(CC(=O)NC1Cc2ccccc2C1)C(=O)c1cc2ccccc2[nH]1)Nc1ccc(Oc2ccc(F)cc2)cc1] | ¹H NMR (500 MHz, DMSO): δ 2.77-2.86 (m, 2H), 3.17-3.25 (m, 2H), 4.24-4.30 (m, 2H), 4.49-4.60 (m, 3H), 6.74 (s, 1 H), 6.98-7.69 (m, 16 H), 8.74-9.02 (m, 1H), 10.65, 11.21 (2 s, 1H), 11.65 (s, 1H), chemical formula: C₃₄H₂₉FN₄O₄ m/z Calcd for [M+ H]⁺ 577.2, m/z found 577.4. |
| 201-11 | 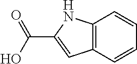 | 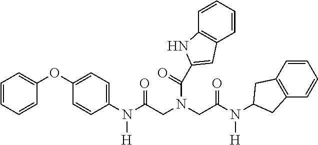 [SMILES: O=C(CN(CC(=O)NC1Cc2ccccc2C1)C(=O)c1cc2ccccc2[nH]1)Nc1ccc(Oc2ccccc2)cc1] | ¹H NMR (500 MHz, DMSO): δ 2.77-3.23 (m, 4H), 4.24-4.61 (m, 5H), 6.60-7.70 (m, 18H), 8.74-9.02 (m, 1H), 10.66-11.21 (2s, 1H), 11.66 (s, 1H), chemical formula: C₃₄H₃₀N₄O₄ m/z Calcd for [M+ H]⁺ 559.23, m/z found 559.5. |
| 201-13 | 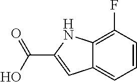 | 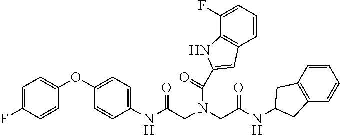 [SMILES: O=C(CN(CC(=O)NC1Cc2ccccc2C1)C(=O)c1cc2cccc(F)c2[nH]1)Nc1ccc(Oc2ccc(F)cc2)cc1] | ¹H NMR (500 MHz, DMSO): δ 2.51-3.35 (m, 4H), 4.21-4.58 (m, 5H), 6.81-7.68 (m, 16 H), 8.79-9.07 (m, 1H), 10.79-11.18 (m, 1H), 12.17 (s, 1H), chemical formula: C₃₄H₂₈F₂N₄O₄ m/z Calcd for [M+ H]⁺ 595.2, found 595.4. |
| 201-14 | 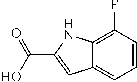 | 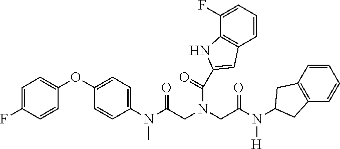 [SMILES: CN(C(=O)CN(CC(=O)NC1Cc2ccccc2C1)C(=O)c1cc2cccc(F)c2[nH]1)c1ccc(Oc2ccc(F)cc2)cc1] | ¹H NMR (500 MHz, DMSO): δ 2.69-3.32 (m, 7H), 4.01-4.53 (m, 5H), 6.61-7.54 (m, 16H), 8.47-8.98 (m, 1H), 12.08-12.14 (m, 1H), chemical formula: C₃₅H₃₀F₂N₄O₄ m/z Calcd for [M+ H]⁺ 609.23, m/z found 609.44. |
| 201-15 | 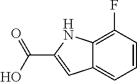 | 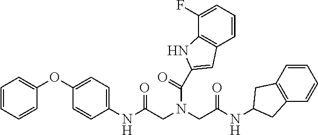 [SMILES: O=C(CN(CC(=O)NC1Cc2ccccc2C1)C(=O)c1cc2cccc(F)c2[nH]1)Nc1ccc(Oc2ccccc2)cc1] | ¹H NMR (500 MHz, DMSO): δ 2.74-3.33 (m, 4H), 4.22-4.58 (m, 5H), 6.81-7.66 (m, 17 H), 8.74-8.99 (m, 1H), 10.69, 11.10 (2s, 1H), 12.17 (s, 1H) chemical formula: C₃₄H₂₉FN₄O₄ m/z Calcd for [M+ H]⁺ 577.2, m/z found 577.4. |

### Alternative General Procedure for the Preparations of the Compounds of the Present Disclosure

An aldehyde (2 mmol) was added to the amine (2 mmol) in DCE (5 mL) and was stirred for 15 min at room temperature. To the resulting mixture NaBH(OAC)₃ (2.5 mmol) was added and then the mixture was stirred under room temperature until TLC showed complete disappearance of the starting aldehyde. The reaction mixture was quenched with water (10 mL) and extracted with CH₂Cl₂ (3×10 mL). The combined extract was dried over anhydrous Na₂SO₄, filtered and concentrated. The residue was purified by MPLC to furnish the desired products.

### Preparation of Compound 201-135

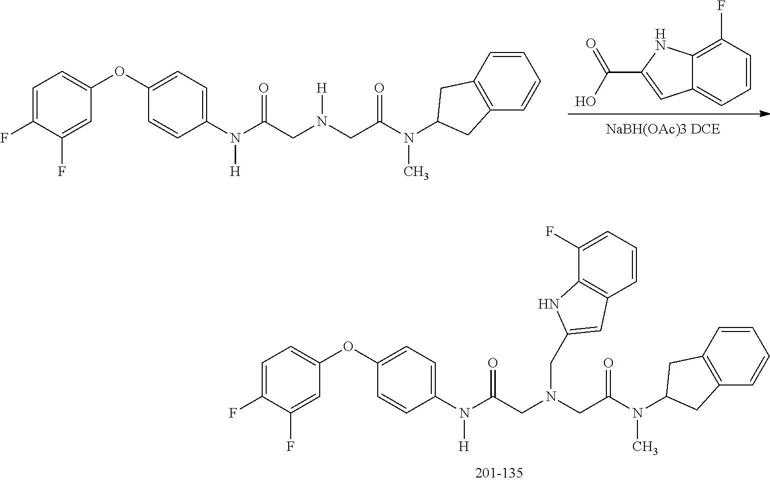 [SMILES: CN(C(=O)CNCC(=O)Nc1ccc(Oc2ccc(F)c(F)c2)cc1)C1Cc2ccccc2C1 ; O=C(O)c1cc2cccc(F)c2[nH]1 ; CN(C(=O)CN(CC(=O)Nc1ccc(Oc2ccc(F)c(F)c2)cc1)Cc1cc2cccc(F)c2[nH]1)C1Cc2ccccc2C1]

Compound 201-135 was prepared using the general procedure for the preparations of compounds of the present disclosure. Some other compounds were prepared according to the synthetic procedure of the compound 201-135 but using different aldehydes and diamide intermediates.

TABLE 5

| Com- | | | |
|---|---|---|---|
| pound | | | |
| No. | Aldehyde | Final compound | Identification data |
| 201-135 | 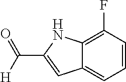 | 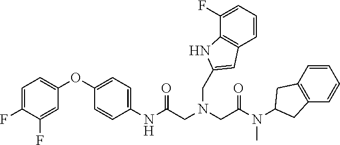 [SMILES: CN(C(=O)CN(CC(=O)Nc1ccc(Oc2ccc(F)c(F)c2)cc1)Cc1cc2cccc(F)c2[nH]1)C1Cc2ccccc2C1] | ¹H NMR (500 MHz, DMSO): δ 2.49-3.08 (m, 7H), 3.32-4.06 (m, 6H), 4.74-5.38 (m, 1H), 6.46 (s, 1H), 6.75-7.68 (m, 14H), 10.38, 10.47 (2s, 1H), 11.53-11.55 (m, 1H), chemical formula: C₃₅H₃₁F₃N₄O₃ m/z Calcd for [M+ H]⁺ 613.2, m/z found 613.3. |
| 201-140 | 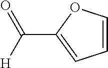 | 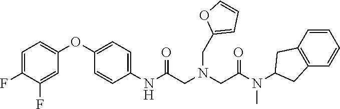 [SMILES: CN(C(=O)CN(CC(=O)Nc1ccc(Oc2ccc(F)c(F)c2)cc1)Cc1ccco1)C1Cc2ccccc2C1] | ¹H NMR (500 MHz, DMSO): δ 2.71-3.14 (m, 7H), 3.31-3.88 (m, 6H), 4.75-5.39 (m, 1H), 6.37-7.67 (m, 14H), 10.43, 10.57 (2s, 1H), chemical formula: C₃₁H₂₉F₂N₃O₄ m/z Calcd for [M+ H]⁺ 546.2, m/z found 546.3. |
| 201-142 | 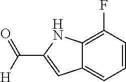 | 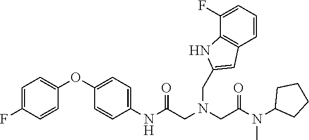 [SMILES: CN(C(=O)CN(CC(=O)Nc1ccc(Oc2ccc(F)cc2)cc1)Cc1cc2cccc(F)c2[nH]1)C1CCCC1] | ¹H NMR (500 MHz, DMSO): δ 1.41-1.63 (m, 8H), 2.69, 2.73 (2s, 3H), 3.45-4.83 (m, 7H), 6.44 (s, 1H), 6.83-7.65 (m, 11H), 10.32, 10.44 (2s, 1H), 11.53 (s, 1H), chemical formula: C₃₁H₃₂F₂N₄O₃ m/z Calcd for [M+ H]⁺ 547.2, m/z found 547.3. |
| 201-144 | 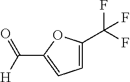 | 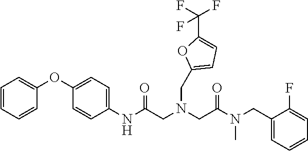 [SMILES: CN(Cc1ccccc1F)C(=O)CN(CC(=O)Nc1ccc(Oc2ccccc2)cc1)Cc1ccc(C(F)(F)F)o1] | ¹H NMR (500 MHz, DMSO): δ 2.82, 2.92 (2s, 3H), 3.44-4.58 (m, 8H), 6.53-6.62 (m, 1H), 6.94-7.63 (m, 14H), 10.38-10.40 (m, 1H), chemical formula: C₃₀H₂₇F₄N₃O₄ m/z Calcd for [M+ H]⁺ 570.2, m/z found 570.3. |

### Preparation of Compound 201-255

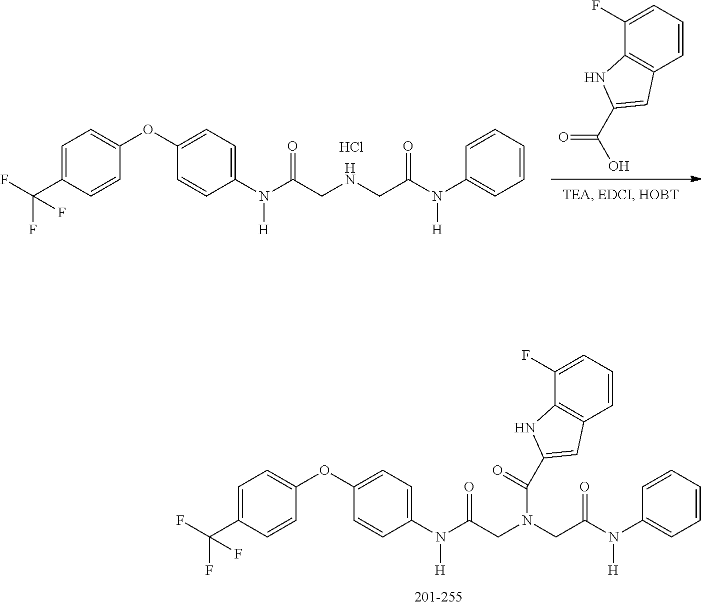

Compound 201-255 was prepared using the general procedure for the preparations of compounds of the present disclosure. Some other compounds were prepared according to the synthetic procedure of the compound 201-255 but using different aldehydes and diamide intermediates.

TABLE 6

| Com- | | | |
|---|---|---|---|
| pound | | | |
| No. | Aldehyde | Final compound | Identification data |
| 201-255 | 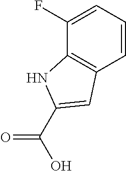 | 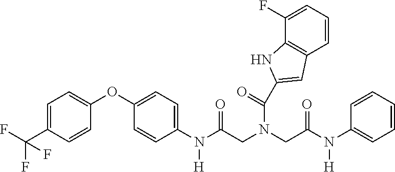 [SMILES: O=C(CN(CC(=O)Nc1ccc(Oc2ccc(C(F)(F)F)cc2)cc1)C(=O)c1cc2cccc(F)c2[nH]1)Nc1ccccc1] | ¹H NMR (500 MHz, DMSO): δ 4.41-4.67 (m, 4H), 6.85-7.74 (m, 17H), 10.39-10.73 (m, 2H), 12.19 (s, 1H), chemical formula: C₃₂H₂₄F₄N₄O₄ m/z Calcd for [M+ H]⁺ 605.17, m/z found 605.1. |
| 201-256 | 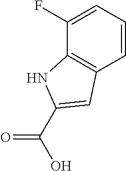 | 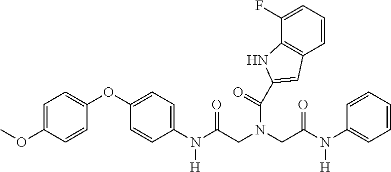 [SMILES: COc1ccc(Oc2ccc(NC(=O)CN(CC(=O)Nc3ccccc3)C(=O)c3cc4cccc(F)c4[nH]3)cc2)cc1] | ¹H NMR (500 MHz, DMSO): δ 3.74 (s, 3H), 4.38-4.64 (m, 4H), 6.84-7.66 (m, 17H), 10.37-10.63 (m, 2H), 12.18 (s, 1H), chemical formula: C₃₂H₂₇FN₄O₅ m/z Calcd for [M+ H]⁺ 567.20, m/z found 567.0. |
| 201-258 | 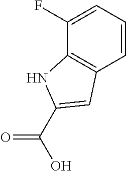 | 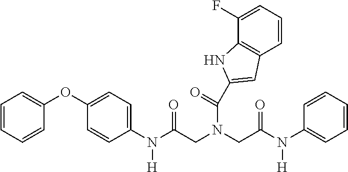 [SMILES: O=C(CN(CC(=O)Nc1ccc(Oc2ccccc2)cc1)C(=O)c1cc2cccc(F)c2[nH]1)Nc1ccccc1] | ¹H NMR (500 MHz, DMSO): δ 4.40-4.66 (m, 4H), 6.85-7.68 (m, 18H), 10.39-10.65 (m, 2H), 12.19 (s, 1H), chemical formula: C₃₁H₂₅FN₄O₄ m/z Calcd for [M+ H]⁺ 537.19, m/z found 537.1. |
| 201-261 | 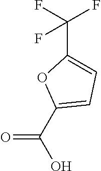 | 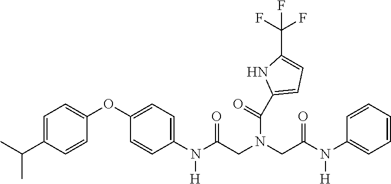 [SMILES: CC(C)c1ccc(Oc2ccc(NC(=O)CN(CC(=O)Nc3ccccc3)C(=O)c3ccc(C(F)(F)F)[nH]3)cc2)cc1] | ¹H NMR (500 MHz, DMSO): δ 1.18-1.21 (m, 6H), 2.83-2.92 (m, 1H), 4.36-4.58 (m, 4H), 6.89-7.63 (m, 15H), 10.36-10.48 (2s, 2H), chemical formula: C₃₁H₂₈F₃N₃O₅ m/z Calcd for [M+ H]⁺ 580.20, m/z found 580.0. |
| 201-263 | 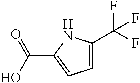 | 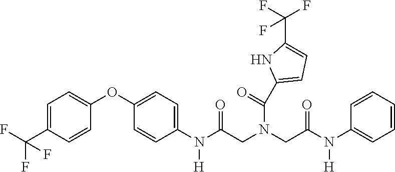 [SMILES: O=C(CN(CC(=O)Nc1ccc(Oc2ccc(C(F)(F)F)cc2)cc1)C(=O)c1ccc(C(F)(F)F)[nH]1)Nc1ccccc1] | ¹H NMR (500 MHz, DMSO): δ 4.36-4.59 (m, 4H), 6.48-7.73 (m, 15H), 10.36-10.75 (m, 2H), 12.90 (s, 1H) chemical formula: C₂₉H₂₂F₆N₄O₄ m/z Calcd for [M+ H]⁺ 605.15, m/z found 605.0. |
| 201-265 | 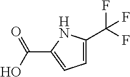 | 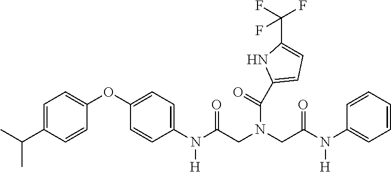 [SMILES: CC(C)c1ccc(Oc2ccc(NC(=O)CN(CC(=O)Nc3ccccc3)C(=O)c3ccc(C(F)(F)F)[nH]3)cc2)cc1] | ¹H NMR (500 MHz, DMSO): δ 1.18-1.21 (m, 6H), 2.83-2.92 (m, 1H), 4.35-4.57 (m, 4H), 6.47-7.65 (m, 15H), 10.38-10.65 (2s, 2H), 12.89 (s, 1H) chemical formula: C₃₁H₂₉F₃N₄O₄ m/z Calcd for [M+ H]⁺ 579.21, m/z found 579.0. |
| 201-267 | 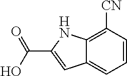 | 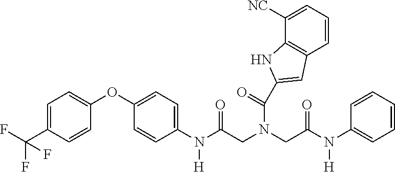 [SMILES: N#Cc1cccc2cc(C(=O)N(CC(=O)Nc3ccccc3)CC(=O)Nc3ccc(Oc4ccc(C(F)(F)F)cc4)cc3)[nH]c12] | ¹H NMR (500 MHz, DMSO): δ 4.40-4.63 (m, 4H), 6.94-8.03 (m, 17H), 10.37-10.66 (m, 2H), 12.65 (s, 1H) chemical formula: C₃₃H₂₄F₃N₅O₄ m/z Calcd for [M+ H]⁺ 612.18, m/z found 612.0. |
| 201-269 | 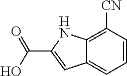 | 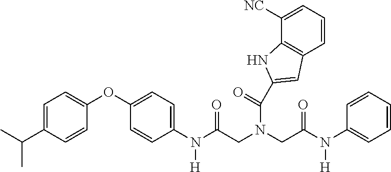 [SMILES: CC(C)c1ccc(Oc2ccc(NC(=O)CN(CC(=O)Nc3ccccc3)C(=O)c3cc4cccc(C#N)c4[nH]3)cc2)cc1] | ¹H NMR (500 MHz, DMSO): δ 1.17-1.23 (m, 6H), 2.82-2.91 (m, 1H), 4.39-4.61 (m, 4H), 6.87-8.02 (m, 17H), 10.38-10.56 (2s, 2H), 12.64 (s, 1H) chemical formula: C₃₅H₃₁N₅O₄ m/z Calcd for [M+ H]⁺ 586.24, m/z found 586.1. |
| 201-274 | 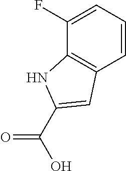 | 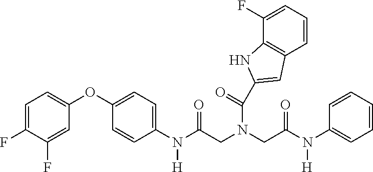 [SMILES: O=C(CN(CC(=O)Nc1ccc(Oc2ccc(F)c(F)c2)cc1)C(=O)c1cc2cccc(F)c2[nH]1)Nc1ccccc1] | ¹H NMR (500 MHz, DMSO): δ 4.40-4.66 (m, 4H), 6.79-7.70 (m, 16H), 10.39-10.68 (m, 2H), 12.19 (s, 1H), chemical formula: C₃₁H₂₃F₃N₄O₄ m/z Calcd for [M+ H]⁺ 573.17, m/z found 573.0. |

Biological Assay

Materials and Methods:

Cell Culture:

HepG2 cells (ATCC, Cat.: HB-8065) were maintained in Growth medium-Eagle's Minimum Essential Medium (Corning, 10-010-CVR), 10% FBS (Gibco, 10099-141), Penicillin (100 units/mL), Streptomycin (100 g/mL). HepG2 cells were incubated at 37° C., 5% CO₂.

Cell Viability Assay

HepG2 cells were plated in black clear bottom 96-well plates (Corning, 3063) at 40,000 cells/well in 100 μL of growth media. After an overnight incubation, the culture media were changed to serum-free OptiMEM media (Gibco, 31985-062), 90 μL/well. Vehicle, PF-06446846 hydrochloride, berberine, or test compound was added to the culture media, 10 μL/well. After 24 hr cellular ATP levels were measured using CellTiter-Glo® 2.0 Assay (Promega, G9242).

ELISA Assays

HepG2 cells were plated in flat bottom 96-well plates (Corning, 3599) at 40,000 cells/well in 100 μL of growth media. After an overnight incubation, the culture media were changed to serum-free OptiMEM media (Gibco, 31985-062), 90 μL/well. Vehicle, PF-06446846 hydrochloride, berberine, or test compound was added to the culture media, 10 μL/well. After 24 hr medium was harvested, and 10 μL of the medium were used for the PCSK9 ELISA (R&D Systems, SPC900).

RNA Extraction and Reverse Transcription Quantitative PCR(RT-Q-PCR) Analysis

Cells were cultured in growth medium as described above and treated with the vehicle, Berberine or test compound for 24 h. The RNA was extracted using the Total RNA mini Kit (Tiangen, Beijing, China) according to the manufacturer's instructions. Reverse transcription was carried out using the High-Capacity cDNA reverse transcription kit (Thermo Fisher Scientific). Quantitative real-time PCR was performed using a reaction mixture containing cDNA, specific primers [PCSK9, 5′-GCTGAGCTGCTCCAGTTTCT-3′ (forward) and 5′-AATGGCGTAGACACCCTCAC-3′ (reverse); GAPDH, 5′-CATGAGAAGTATGACAACAGCCT-3′ (forward) and 5′-AGTCCTTCCACGATACCAAAGT-3′ (reverse)] and Maxima SYBR Green/ROX qPCR Master Mix (Thermo Fisher Scientific). PCR amplification was carried out in a Real-Time PCR System. The real-time PCR conditions were 37° C. 10 min; 95° C. 10 min; 95° C. 15 s, 60° C. 30 s, 72° C. 30 s, 40 cycle. The amount of % RNA was normalized to the GAPDH level in the same samples.

TABLE 5

| | HepG2 Survival | HepG2 PCSK9 | HepG2 Q-PCR |
|---|---|---|---|
| Compound | assay | ELISA (10 μM) | PCSK9 mRNA |
| No. | (10 μM) | % inhibition | level decrease (10 μM) |
| Berberine | | 52% | 35% |
| 201-11 | 100% | 38% | 79.30% |
| 201-13 | 97% | 64% | 93.30% |
| 201-14 | 93% | 41% | 89.50% |
| 201-15 | 100% | 46% | 95.20% |
| 201-29 | 100% | 61% | 88.50% |
| 201-31 | 100% | 48% | 73% |
| 201-37 | 100% | 50% | 63% |
| 201-41 | 100% | 72% | 75% |
| 201-43 | 100% | 60% | 77% |
| 201-45 | 100% | 60% | 80% |
| 201-46 | 100% | 33% | 46% |
| 201-47 | 99% | 70% | 90% |
| 201-49 | 100% | 50% | 77% |
| 201-50 | 100% | 47% | 77% |
| 201-51 | 93% | 55% | 75% |
| 201-52 | 100% | 45% | 79% |
| 201-53 | 100% | 50% | 80% |
| 201-54 | 100% | 52% | 61% |
| 201-65 | 100% | 85% | 87% |
| 201-69 | 92% | 80% | 78% |
| 201-70 | 100% | 76% | 85% |
| 201-74 | 96% | 77% | 88% |
| 201-75 | 100% | 80% | 90% |
| 201-80 | 100% | 80% | 88% |
| 201-85 | 100% | 46% | 70% |
| 201-91 | 100% | 40% | 58% |
| 201-97 | 100% | 48% | 37% |
| 201-103 | 100% | 86% | 85% |
| 201-104 | 100% | 83% | 86% |
| 201-109 | 100% | 92% | 90% |
| 201-110 | 100% | 84% | 88% |
| 201-115 | 100% | 90% | 90% |
| 201-116 | 100% | 81% | 88% |
| 201-127 | 90% | 74% | 92% |
| 201-133 | 100% | 98% | 92% |
| 201-134 | 100% | 90% | 92% |
| 201-205 | 90% | 85% | 92% |
| 201-209 | 92% | 91% | — |
| 201-227 | 112% | 71% | 56% |
| 201-235 | 101% | 73% | 72% |
| 201-237 | 98% | 72% | 77% |
| 201-242 | 115% | 74% | 77% |
| 201-245 | 101% | 76% | — |
| 201-249 | 97% | 85% | — |
| 201-253 | 110% | 61% | 59% |
| 201-255 | 101% | 87% | 72% |
| 201-256 | 109% | 78% | 75% |
| 201-258 | 97% | 69% | — |
| 201-261 | 99% | 60% | — |
| 201-263 | 88% | 84% | — |
| 201-265 | 93% | 71% | 76% |
| 201-267 | 103% | 56% | 79% |
| 201-269 | 106% | 53% | 87% |
| 201-274 | 98% | 77% | 88% |

Measurement of Dil-LDL Uptake

HepG2 cells were maintained in MEM supplemented with 10% FBS. The cells were seeded in 96 well black plates at a density of 1×10⁴ cells per well and grown to 70-80% confluence. Afterwards, cells were changed to serum-free Opti-MEM for 1 h and followed by incubation with 10 μM Berberine or compounds of the present disclosure for 20 h. Then, 20 μg/mL Dil-LDL was added and incubated at 37° C. in the dark for additional 4 h. Cells were extensively washed three times with PBS, and LDL uptake was determined on a fluorescence plate reader at an excitation wavelength of 520 nm and emission wavelength of 580 nm.

| | LDL uptake 10 μM (fold |
|---|---|
| Compound No. | of vehicle) |
| Berberine | 2.54 fold |
| 201-29 | 3.07 fold |
| 201-31 | 2.07 fold |
| 201-65 | 1.84 fold |
| 201-70 | 1.69 fold |
| 201-74 | 3.92 fold |
| 201-75 | 2.92 fold |
| 201-80 | 3.07 fold |
| 201-104 | 4.15 fold |
| 201-109 | 4.69 fold |
| 201-110 | 4.76 fold |
| 201-115 | 4.07 fold |
| 201-116 | 3.76 fold |
| 201-134 | 5.30 fold |
| 201-205 | 1.1 fold |
| 201-227 | 1.37 fold |
| 201-242 | 2.43 fold |
| 201-245 | 1.11 fold |
| 201-253 | 1.77 fold |
| 201-256 | 2.43 fold |
| 201-265 | 1.57 fold |
| 201-267 | 1.34 fold |
| 201-269 | 1.34 fold |

While the present disclosure has been described in detail with reference to the specific preferred embodiments, it cannot be concluded that the specific embodiments of the present disclosure are limited to these descriptions. Those skilled in the art will appreciate that several simple deductions or substitutions may be made without departing from the spirit of the present disclosure, which should be regarded to be within the scope of the present disclosure.

1. A compound of formula (I), or a pharmaceutically acceptable salt, an enantiomer, a diastereomer, a racemate, a solvate, a hydrate, a polymorph, a prodrug, or an isotope variant thereof, and mixtures thereof: 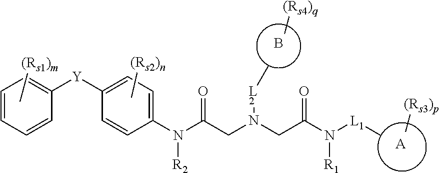
wherein:
Ring A is selected from C₃₋₇ cycloalkyl, 3- to 7-membered heterocyclyl, or C₆₋₁₀ aryl;
Ring B is selected from C₃₋₇ cycloalkyl, 3- to 7-membered heterocyclyl, C₆₋₁₀ aryl, or 5- to 10-membered heteroaryl;
L₁ is selected from a bond, —O—, —C(O)—, —CR′R″—, —CR′R″—CR′R″—, or —CR′R″—CR′R″—CR′R″—;
L₂ is selected from a bond, —C(O)—, —CR′R″—, —CR′R″—CR′R″—, or —CR′R″—CR′R″—CR′R″—;
Y is selected from O, S, NH, or CH₂;
R₁ is selected from H, —C(O)Rₐ, —C(O)ORₐ, —C(O)NR_{b}R_{c}, C₁₋₆ alkyl, C₁₋₆ haloalkyl, C₃₋₇ cycloalkyl, 3- to 7-membered heterocyclyl, C₆₋₁₀ aryl, or 5- to 10-membered heteroaryl;
R₂ is selected from H, C₁₋₆ alkyl, or C₁₋₆ haloalkyl;
Rₛ₁ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
Rₛ₂ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
Rₛ₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
Rₛ₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
m=0, 1, 2, 3, 4, or 5;
n=0, 1, 2, 3, or 4;
p=0, 1, 2, 3, 4, 5, 6, 7, or 8;
q=0, 1, 2, 3, 4, or 5;
and wherein,
R′ and R″ are each independently selected from H, halogen, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, or C₂₋₆ alkynyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
wherein each of Y, R₁, R₂, Rₛ₁, Rₛ₂, Rₛ₃, and Rₛ₄ is optionally substituted by 1, 2 or 3 R groups, wherein R is independently selected from H, —OH, halogen, —NO₂, carbonyl, -L-CN, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—NR_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, -L-O—C(S)—NR_{b}R_{c}, C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl; wherein the said C₁₋₆ alkyl, C₁₋₆ haloalkyl, C₂₋₆ alkenyl, C₂₋₆ alkynyl, -L-C₃₋₇ cycloalkyl, -L-3- to 7-membered heterocyclyl, -L-C₆₋₁₀ aryl, or -L-5- to 10-membered heteroaryl is each further optionally substituted by one or more groups consisting of the following:
-L-CN, —NO₂, carbonyl, -L-ORₐ, -L-SRₐ, -L-NR_{b}R_{c}, -L-C(O)Rₐ, -L-C(S)Rₐ, -L-C(O)ORₐ, -L-C(S)ORₐ, -L-C(O)—NR_{b}R_{c}, -L-C(S)—NR_{b}R_{c}, -L-O—C(O)Rₐ, -L-O—C(S)Rₐ, -L-N(R_{b})—C(O)—Rₐ, -L-N(R_{b})—C(S)—Rₐ, -L-S(O)ₓRₐ, -L-S(O)ₓORₐ, -L-S(O)ₓNR_{b}R_{c}, -L-N(R_{b})—S(O)ₓ—Rₐ, -L-N(R_{b})—S(O)ₓ—R_{b}R_{c}, -L-N(R_{b})—C(O)ORₐ, -L-N(R_{b})—C(S)ORₐ, -L-O—C₁₋₆ alkylene-ORₐ, -L-C(O)—C₁₋₆ alkylene-NR_{b}R_{c}, -L-N(R_{b})—C(O)—NR_{b}R_{c}, -L-N(R_{b})—C(S)—NR_{b}R_{c}, -L-O—C(O)—NR_{b}R_{c}, or -L-O—C(S)—NR_{b}R_{c};
L is selected from a chemical bond, —C₁₋₆ alkylene-, —C₂₋₆ alkenylene- or —C₂₋₆ alkynylene-;
x=0, 1 or 2.

2. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein R₂ is H.

3. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein R₁ is a group other than H.

4. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein q=1, 2, 3, 4, or 5, and at least one of Rₛ₄ is selected from halogen, or C₁₋₆ haloalkyl.

5. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein m=0, 1, 2, or 3, and Rₛ₁ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, or C₁₋₆ haloalkyl.

6. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein p=0, 1, or 2, and Rₛ₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, or C₁₋₆ haloalkyl.

7. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein Y is O.

8. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein L₂ is —C(O)—.

9. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein Ring A is selected from the following:

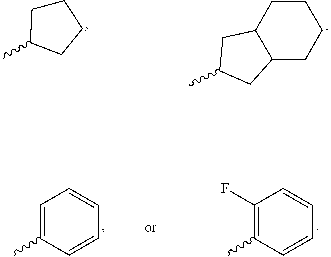

10. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein Ring B is selected from the following: 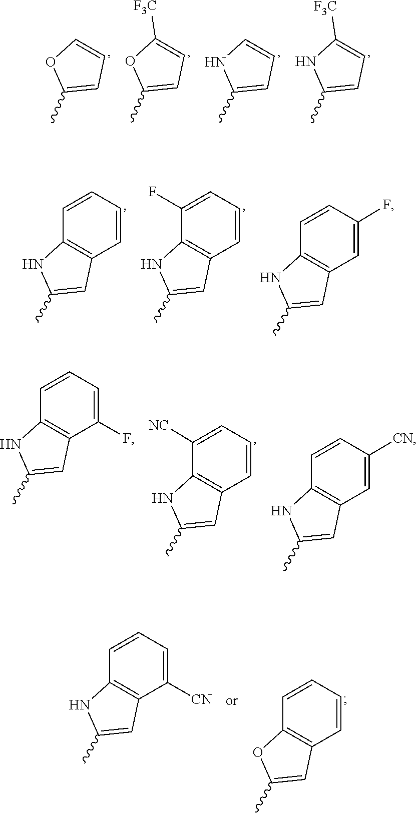
preferably, wherein Ring B is selected from the following:

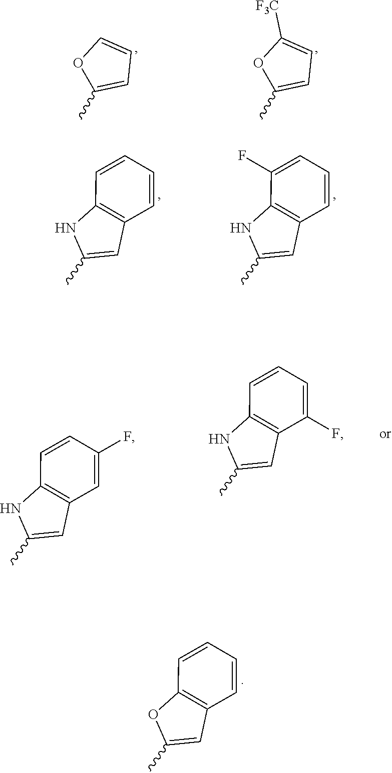

11. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formulae (II-1) to (II-4): 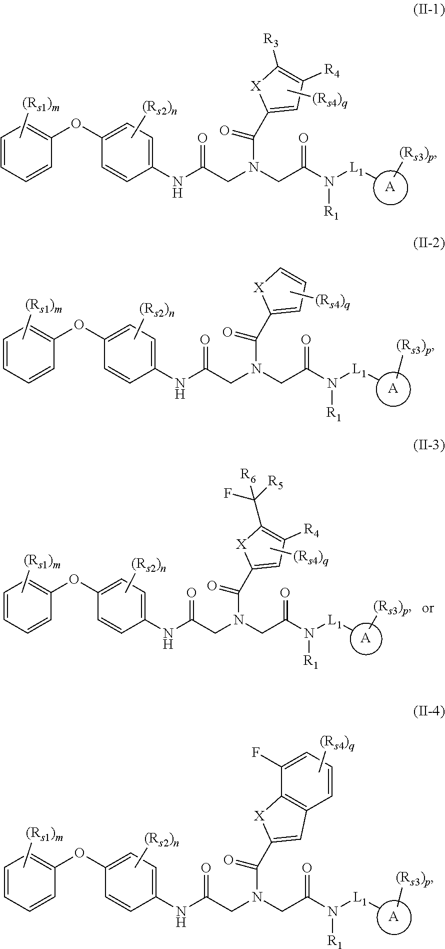
wherein:
X is selected from O, S, NH or CH₂;
R₃ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R₃ and R₄ are linked to form a C₃₋₇ cycloalkyl, 3- to 7-membered heterocyclyl, C₆₋₁₀ aryl, or 5- to 10-membered heteroaryl;
R₅ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;
R₆ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;
or, R₅ and R₄ are linked to form a C₃₋₇ cycloalkyl, 3- to 7-membered heterocyclyl, C₆₋₁₀ aryl, or 5- to 10-membered heteroaryl; and
other variables are as defined in claim 1.

12. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formulae (III-1) to (III-3): 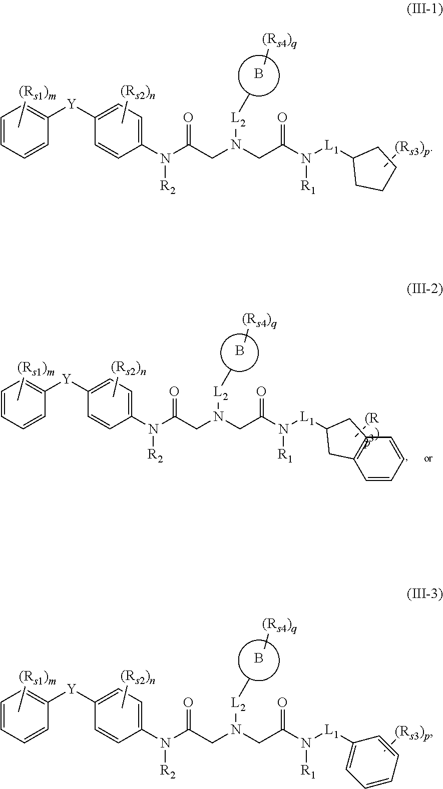
wherein the variables are as defined in claim 1.

13. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formulae (IV-1) to (IV-3): 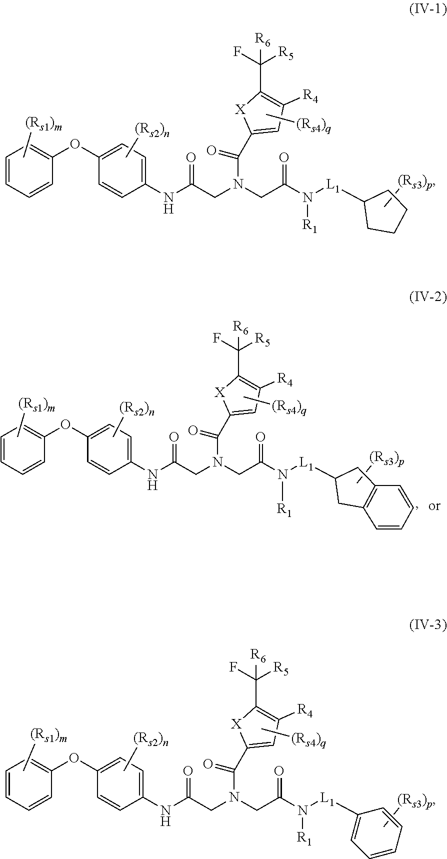
wherein:
X is selected from O, S, or NH;
R₄ is selected from H, halogen, —CN, —NO₂, —ORₐ, —SRₐ, —NR_{b}R_{c}, —C(O)Rₐ, —C(O)ORₐ, —C(O)NR_{b}R_{c}, C₁₋₆ alkyl, C₁₋₆ haloalkyl, C₂₋₆ alkenyl, or C₂₋₆ alkynyl;
R₅ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;
R₆ is selected from H, halogen, —CN, —NO₂, —ORₐ, —SRₐ, —NR_{b}R_{c}, C₁₋₆ alkyl, C₁₋₆ haloalkyl, C₂₋₆ alkenyl, or C₂₋₆ alkynyl;
or, R₅ and R₄ are linked to form a C₆₋₁₀ aryl, or 5- to 10-membered heteroaryl; and
other variables are as defined in claim 1.

14. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formula (III-1): 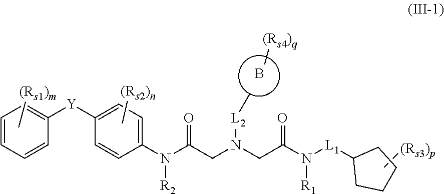
wherein,
Ring B is 5- to 10-membered heteroaryl;
L₁ is a bond;
L₂ is selected from a bond, —C(O)—, or —CR′R″—;
Y is selected from O, S, or NH;
R₁ is selected from H, C₁₋₆ alkyl, or C₁₋₆ haloalkyl;
R₂ is selected from H, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
R′ and R″ are each independently selected from H, halogen, C₁₋₆ alkyl, or C₁₋₆ haloalkyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
Ring B is selected from the following: 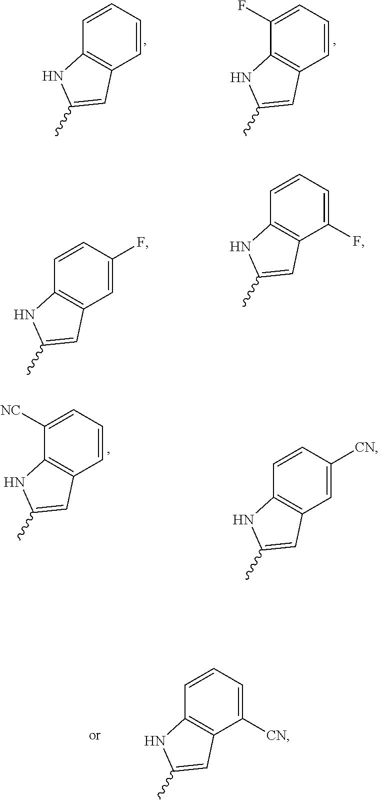
L₁ is a bond;
L₂ is —C(O)—;
Y is O;
R₁ is selected from H, C₁₋₆ alkyl, or C₁₋₆ haloalkyl;
R₂ is H;
Rₛ₁ is selected from H, halogen, C₁₋₆ alkyl, or C₁₋₆ haloalkyl;
Rₛ₂ is H;
Rₛ₃ is H;
Rₛ₄ is H;
m=0, 1, or 2;
n=0, 1, or 2;
p=0, 1, or 2;
q=0, 1, or 2.

15. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formula (III-2): 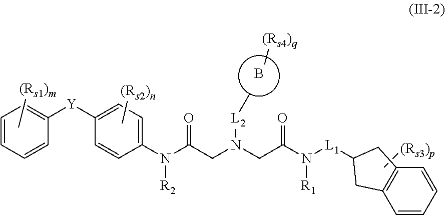
wherein,
Ring B is 5- to 10-membered heteroaryl;
L₁ is a bond;
L₂ is selected from a bond, —C(O)—, or —CR′R″—;
Y is selected from O, S, or NH;
R₁ is selected from H, C₁₋₆ alkyl, or C₁₋₆ haloalkyl;
R₂ is selected from H, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
R′ and R″ are each independently selected from H, halogen, C₁₋₆ alkyl, or C₁₋₆ haloalkyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
Ring B is selected from the following: 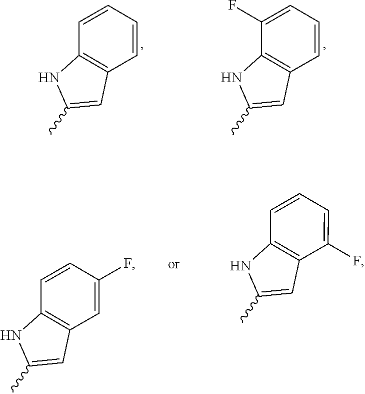
L₁ is a bond;
L₂ is —C(O)—;
Y is O;
R₁ is selected from H, C₁₋₆ alkyl, or C₁₋₆ haloalkyl;
R₂ is selected from H, C₁₋₆ alkyl, or C₁₋₆ haloalkyl;
Rₛ₁ is selected from H, or halogen;
Rₛ₂ is H;
Rₛ₃ is H;
Rₛ₄ is H;
m=0, 1, or 2;
n=0, 1, or 2;
p=0, 1, or 2;
q=0, 1, or 2;
alternatively,
Ring B is 5- to 6-membered heteroaryl;
L₁ is a bond;
L₂ is selected from a bond, —C(O)—, or —CR′R″—;
Y is selected from O, S, or NH;
R₁ is C₁₋₆ alkyl, or C₁₋₆ haloalkyl;
R₂ is H;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
Ring B is selected from the following: 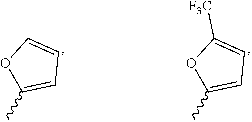
L₁ is a bond;
L₂ is —C(O)—;
Y is O;
R₁ is C₁₋₆ alkyl, or C₁₋₆ haloalkyl;
R₂ is H;
Rₛ₁ is selected from H, or halogen;
Rₛ₂ is H;
Rₛ₃ is H;
Rₛ₄ is H;
m=0, 1, or 2;
n=0, 1, or 2;
p=0, 1, or 2;
q=0, 1, or 2.

16. The compound of formula (I), or a pharmaceutically acceptable salt, an enantiomer, a diastereomer, a racemate, a solvate, a hydrate, a polymorph, a prodrug, or an isotope variant thereof, and mixtures thereof according to a claim 1, which is the compound of formula (III-3): 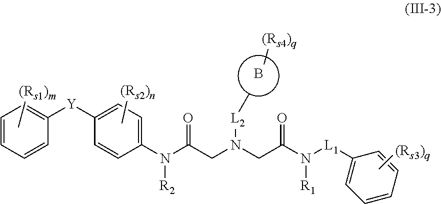
wherein:
Ring B is 5- to 10-membered heteroaryl;
L₁ is —CR′R″—, —CR′R″—CR′R″—, or —CR′R″—CR′R″—CR′R″—;
L₂ is selected from a bond, —C(O)—, or —CR′R″—;
Y is selected from O, S, or NH;
R₁ is selected from H, C₁₋₆ alkyl, or C₁₋₆ haloalkyl;
R₂ is selected from H, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
R′ and R″ are each independently selected from H, halogen, C₁₋₆ alkyl, or C₁₋₆ haloalkyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
Ring B is selected from the following: 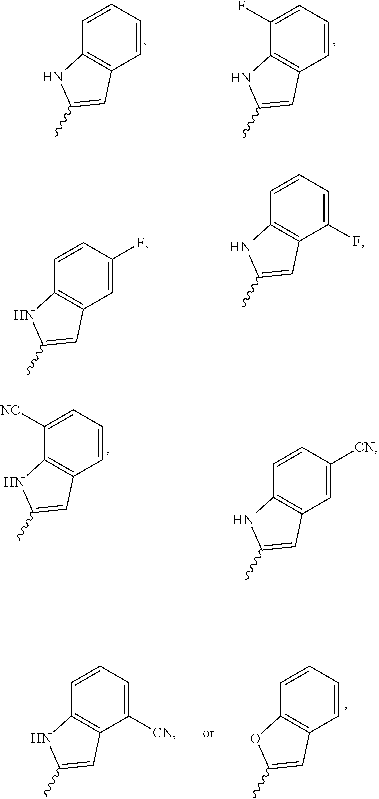
L₁ is —CR′R″—;
L₂ is —C(O)—;
Y is O;
R₁ is selected from H, C₁₋₆ alkyl, or C₁₋₆ haloalkyl;
R₂ is selected from H, C₁₋₆ alkyl, or C₁₋₆ haloalkyl;
Rₛ₁ is selected from H, halogen, C₁₋₆ alkyl, or C₁₋₆ haloalkyl;
Rₛ₂ is H;
Rₛ₃ is H, or halogen;
Rₛ₄ is H;
m=0, 1, or 2;
n=0, 1, or 2;
p=0, 1, or 2;
q=0, 1, or 2;
R′ and R″ are each independently selected from H, or halogen;
alternatively,
Ring B is 5- to 10-membered heteroaryl;
L₁ is a bond;
L₂ is selected from a bond, —C(O)—, or —CR′R″—;
Y is selected from O, S, or NH;
R₁ is H;
R₂ is H;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
Ring B is selected from the following: 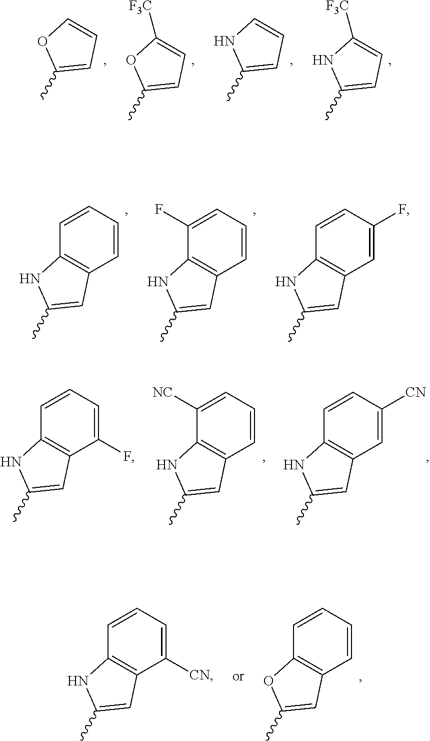
L₁ is a bond;
L₂ is —C(O)—;
Y is O;
R₁ is H;
R₂ is H;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is H;
Rₛ₃ is H;
Rₛ₄ is H;
m=0, 1, or 2;
n=0, 1, or 2;
p=0, 1, or 2;
q=0, 1, or 2;
Rₐ is independently selected from H, C₁₋₆ alkyl, or C₁₋₆ haloalkyl;
R_{b} and R_{c} are each independently selected from H, C₁₋₆ alkyl, or C₁₋₆ haloalkyl;
alternatively,
Ring B is 5- to 6-membered heteroaryl;
L₁ is a —CR′R″—, —CR′R″—CR′R″—, or —CR′R″—CR′R″—CR′R″—;
L₂ is selected from a bond, —C(O)—, or —CR′R″—;
Y is selected from O, S, or NH;
R₁ is C₁₋₆ alkyl, or C₁₋₆ haloalkyl;
R₂ is H;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
R′ and R″ are each independently selected from H, halogen, C₁₋₆ alkyl, or C₁₋₆ haloalkyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
Ring B is selected from the following: 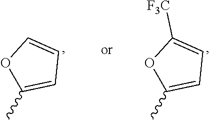
L₁ is a —CR′R″—;
L₂ is —C(O)—;
Y is O;
R₁ is C₁₋₆ alkyl, or C₁₋₆ haloalkyl;
R₂ is H;
Rₛ₁ is selected from H, or halogen;
Rₛ₂ is H;
Rₛ₃ is H, or halogen;
Rₛ₄ is H;
m=0, 1, or 2;
n=0, 1, or 2;
p=0, 1, or 2;
q=0, 1, or 2;
R′ and R″ are each independently selected from H, or halogen.

17. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formula (IV-1): 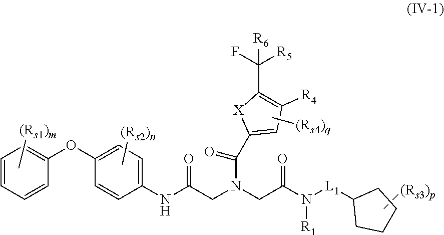
wherein,
X is selected from O, S, or NH;
R₄, R₅ and R₆ are linked together with the atoms they are attached to form a C₆₋₁₀ aryl, or 5- to 10-membered heteroaryl;
L₁ is a bond;
R₁ is C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is NH;
R₄, R₅ and R₆ are linked together with the atoms they are attached to form a phenyl;
L₁ is a bond;
R₁ is C₁₋₆ alkyl, or C₁₋₆ haloalkyl;
Rₛ₁ is selected from H, or halogen;
Rₛ₂ is H;
Rₛ₃ is H;
Rₛ₄ is H;
m=0, 1, or 2;
n=0, 1, or 2;
p=0, 1, or 2;
q=0, 1, or 2;
alternatively,
X is selected from O, S, or NH;
L₁ is a bond;
R₁ is H;
R₄ is selected from H, halogen, —CN, —NO₂, —ORₐ, —SRₐ, —NR_{b}R_{c}, C₁₋₆ alkyl, or C₁₋₆ haloalkyl;
R₅ is selected from H, halogen, —CN, —NO₂, —ORₐ, —SRₐ, —NR_{b}R_{c}, C₁₋₆ alkyl, or C₁₋₆ haloalkyl;
R₆ is selected from H, halogen, —CN, —NO₂, —ORₐ, —SRₐ, —NR_{b}R_{c}, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is NH;
L₁ is a bond;
R₁ is H;
R₄ is selected from H, or halogen;
R₅ is selected from H, or halogen;
R₆ is selected from H, or halogen;
Rₛ₁ is selected from H, halogen, C₁₋₆ alkyl, or C₁₋₆ haloalkyl;
Rₛ₂ is H;
Rₛ₃ is H;
Rₛ₄ is H;
m=0, 1, or 2;
n=0, 1, or 2;
p=0, 1, or 2;
q=0, 1, or 2.

18. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formula (IV-2): 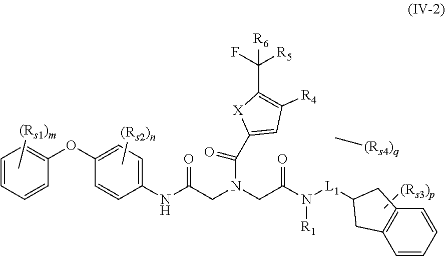
X is selected from O, S, or NH;
R₄, R₅ and R₆ are linked together with the atoms they are attached to form a C₆₋₁₀ aryl, or 5- to 10-membered heteroaryl;
L₁ is a bond;
R₁ is selected from H, C₁₋₆ alkyl, or C₁₋₆ haloalkyl; alternatively, R₁ is H;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is NH;
R₄, R₅ and R₆ are linked together with the atoms they are attached to form a phenyl;
L₁ is a bond;
R₁ is selected from H, C₁₋₆ alkyl, or C₁₋₆ haloalkyl; alternatively, R₁ is H;
Rₛ₁ is selected from H, or halogen;
Rₛ₂ is H;
Rₛ₃ is H;
Rₛ₄ is H;
m=0, 1, or 2;
n=0, 1, or 2;
p=0, 1, or 2;
q=0, 1, or 2;
alternatively,
X is selected from O, S, or NH;
L₁ is a bond;
R₁ is C₁₋₆ alkyl, or C₁₋₆ haloalkyl;
R₄ is selected from H, halogen, —CN, —NO₂, —ORₐ, —SRₐ, —NR_{b}R_{c}, C₁₋₆ alkyl, or C₁₋₆ haloalkyl;
R₅ is selected from H, halogen, —CN, —NO₂, —ORₐ, —SRₐ, —NR_{b}R_{c}, C₁₋₆ alkyl, or C₁₋₆ haloalkyl;
R₆ is selected from H, halogen, —CN, —NO₂, —ORₐ, —SRₐ, —NR_{b}R_{c}, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is O;
L₁ is a bond;
R₁ is C₁₋₆ alkyl, or C₁₋₆ haloalkyl;
R₄ is selected from H, or halogen;
R₅ is selected from H, or halogen;
R₆ is selected from H, or halogen;
Rₛ₁ is selected from H, or halogen;
Rₛ₂ is H;
Rₛ₃ is H;
Rₛ₄ is H;
m=0, 1, or 2;
n=0, 1, or 2;
p=0, 1, or 2;
q=0, 1, or 2.

19. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, which is the compound of formula (IV-3): 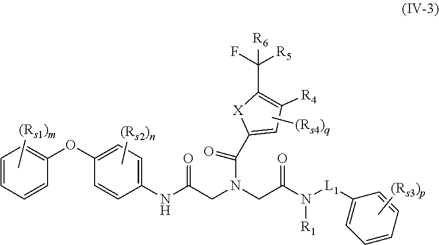
wherein,
X is selected from O, S, or NH;
R₄, R₅ and R₆ are linked together with the atoms they are attached to form a C₆₋₁₀ aryl, or 5- to 10-membered heteroaryl;
L₁ is a —CR′R″—, —CR′R″—CR′R″—, or —CR′R″—CR′R″—CR′R″—;
R₁ is H, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
R′ and R″ are each independently selected from H, halogen, C₁₋₆ alkyl, or C₁₋₆ haloalkyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is NH;
R₄, R₅ and R₆ are linked together with the atoms they are attached to form a phenyl;
L₁ is —CR′R″—;
R₁ is H, C₁₋₆ alkyl, or C₁₋₆ haloalkyl;
Rₛ₁ is selected from H, halogen, C₁₋₆ alkyl, or C₁₋₆ haloalkyl;
Rₛ₂ is H;
Rₛ₃ is H, or halogen;
Rₛ₄ is H;
m=0, 1, or 2;
n=0, 1, or 2;
p=0, 1, or 2;
q=0, 1, or 2;
R′ and R″ are each independently selected from H, or halogen;
alternatively,
X is selected from O, S, or NH;
L₁ is a bond;
R₁ is H;
R₄ is selected from H, halogen, —CN, —NO₂, —ORₐ, —SRₐ, —NR_{b}R_{c}, C₁₋₆ alkyl, or C₁₋₆ haloalkyl;
R₅ is selected from H, halogen, —CN, —NO₂, —ORₐ, —SRₐ, —NR_{b}R_{c}, C₁₋₆ alkyl, or C₁₋₆ haloalkyl;
R₆ is selected from H, halogen, —CN, —NO₂, —ORₐ, —SRₐ, —NR_{b}R_{c}, C₁₋₆ alkyl, or C₁₋₆ haloalkyl;
or R₄, R₅ and R₆ are linked together with the atoms they are attached to form a C₆₋₁₀ aryl, or 5- to 10-membered heteroaryl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is NH;
L₁ is a bond;
R₁ is H;
R₄ is selected from H, or halogen;
R₅ is selected from H, or halogen;
R₆ is selected from H, or halogen;
or R₄, R₅ and R₆ are linked together with the atoms they are attached to form a phenyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is H;
Rₛ₃ is H;
Rₛ₄ is H;
m=0, 1, or 2;
n=0, 1, or 2;
p=0, 1, or 2;
q=0, 1, or 2;
Rₐ is independently selected from H, C₁₋₆ alkyl, or C₁₋₆ haloalkyl;
R_{b} and R_{c} are each independently selected from H, C₁₋₆ alkyl, or C₁₋₆ haloalkyl;
alternatively,
X is selected from O, S, or NH;
L₁ is a —CR′R″—, —CR′R″—CR′R″—, or —CR′R″—CR′R″—CR′R″—;
R₁ is C₁₋₆ alkyl, or C₁₋₆ haloalkyl;
R₄ is selected from H, halogen, —CN, —NO₂, —ORₐ, —SRₐ, —NR_{b}R_{c}, C₁₋₆ alkyl, or C₁₋₆ haloalkyl;
R₅ is selected from H, halogen, —CN, —NO₂, —ORₐ, —SRₐ, —NR_{b}R_{c}, C₁₋₆ alkyl, or C₁₋₆ haloalkyl;
R₆ is selected from H, halogen, —CN, —NO₂, —ORₐ, —SRₐ, —NR_{b}R_{c}, C₁₋₆ alkyl, or C₁₋₆ haloalkyl;
Rₛ₁ is selected from H, halogen, —CN, —NO₂, —ORₐ, —SRₐ, —NR_{b}R_{c}, C₁₋₆ alkyl, or C₁₋₆ haloalkyl;
Rₛ₂ is selected from H, halogen, —CN, —NO₂, —ORₐ, —SRₐ, —NR_{b}R_{c}, C₁₋₆ alkyl, or C₁₋₆ haloalkyl;
Rₛ₃ is selected from H, halogen, —CN, —NO₂, —ORₐ, —SRₐ, —NR_{b}R_{c}, C₁₋₆ alkyl, or C₁₋₆ haloalkyl;
Rₛ₄ is selected from H, halogen, —CN, —NO₂, —ORₐ, —SRₐ, —NR_{b}R_{c}, C₁₋₆ alkyl, or C₁₋₆ haloalkyl;
m=0, 1, 2, or 3;
n=0, 1, 2, or 3;
p=0, 1, 2, or 3;
q=0, 1, 2, or 3;
R′ and R″ are each independently selected from H, halogen, C₁₋₆ alkyl, or C₁₋₆ haloalkyl;
Rₐ is independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
R_{b} and R_{c} are each independently selected from H, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl;
or, R_{b}, R_{c} and N atom are taken together to form 3- to 7-membered heterocyclyl;
alternatively,
X is O;
L₁ is —CR′R″—;
R₁ is C₁₋₆ alkyl, or C₁₋₆ haloalkyl;
R₄ is selected from H, or halogen;
R₅ is selected from H, or halogen;
R₆ is selected from H, or halogen;
Rₛ₁ is selected from H, or halogen;
Rₛ₂ is H;
Rₛ₃ is H, or halogen;
Rₛ₄ is H;
m=0, 1, or 2;
n=0, 1, or 2;
p=0, 1, or 2;
q=0, 1, or 2;
R′ and R″ are each independently selected from H, or halogen.

20. The compound of formula (I), or a pharmaceutically acceptable salt, an enantiomer, a diastereomer, a racemate, a solvate, a hydrate, a polymorph, a prodrug, or an isotope variant thereof, and mixtures thereof according to claim 1, wherein the compound is selected from the group consisting of:

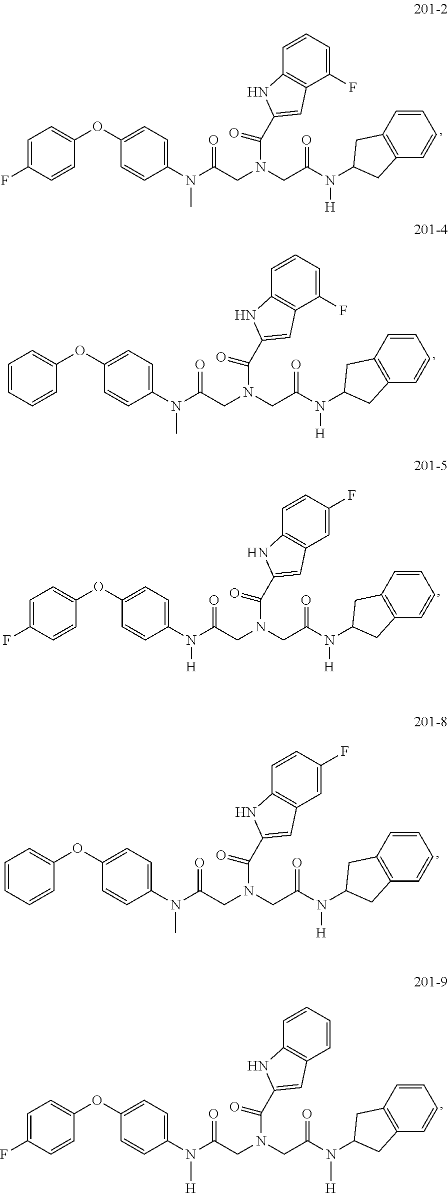 [SMILES: CN(C(=O)CN(CC(=O)NC1Cc2ccccc2C1)C(=O)c1cc2c(F)cccc2[nH]1)c1ccc(Oc2ccc(F)cc2)cc1 ; CN(C(=O)CN(CC(=O)NC1Cc2ccccc2C1)C(=O)c1cc2c(F)cccc2[nH]1)c1ccc(Oc2ccccc2)cc1 ; O=C(CN(CC(=O)NC1Cc2ccccc2C1)C(=O)c1cc2cc(F)ccc2[nH]1)Nc1ccc(Oc2ccc(F)cc2)cc1 ; CN(C(=O)CN(CC(=O)NC1Cc2ccccc2C1)C(=O)c1cc2cc(F)ccc2[nH]1)c1ccc(Oc2ccccc2)cc1 ; O=C(CN(CC(=O)NC1Cc2ccccc2C1)C(=O)c1cc2ccccc2[nH]1)Nc1ccc(Oc2ccc(F)cc2)cc1]

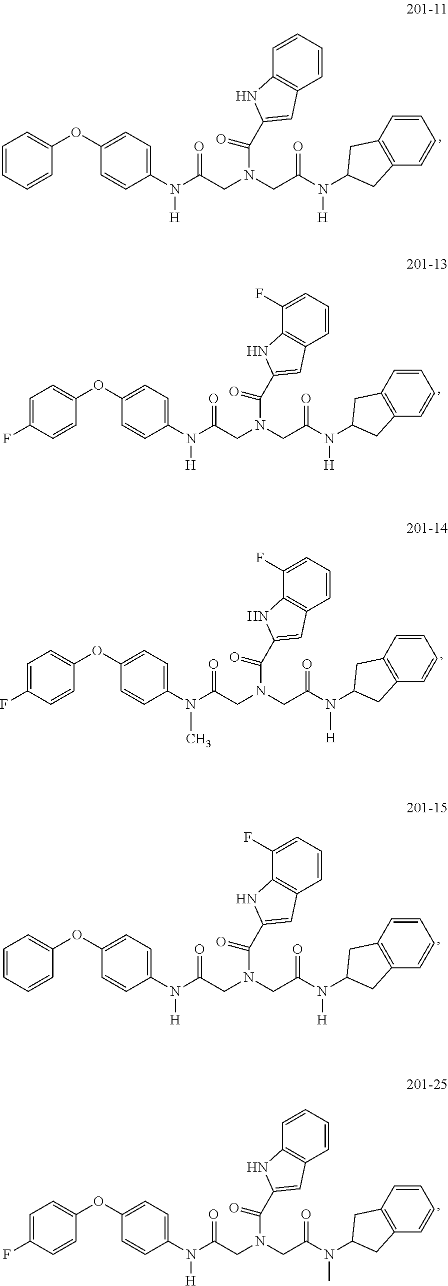 [SMILES: O=C(CN(CC(=O)NC1Cc2ccccc2C1)C(=O)c1cc2ccccc2[nH]1)Nc1ccc(Oc2ccccc2)cc1 ; O=C(CN(CC(=O)NC1Cc2ccccc2C1)C(=O)c1cc2cccc(F)c2[nH]1)Nc1ccc(Oc2ccc(F)cc2)cc1 ; CN(C(=O)CN(CC(=O)NC1Cc2ccccc2C1)C(=O)c1cc2cccc(F)c2[nH]1)c1ccc(Oc2ccc(F)cc2)cc1 ; O=C(CN(CC(=O)NC1Cc2ccccc2C1)C(=O)c1cc2cccc(F)c2[nH]1)Nc1ccc(Oc2ccccc2)cc1 ; CN(C(=O)CN(CC(=O)Nc1ccc(Oc2ccc(F)cc2)cc1)C(=O)c1cc2ccccc2[nH]1)C1Cc2ccccc2C1]

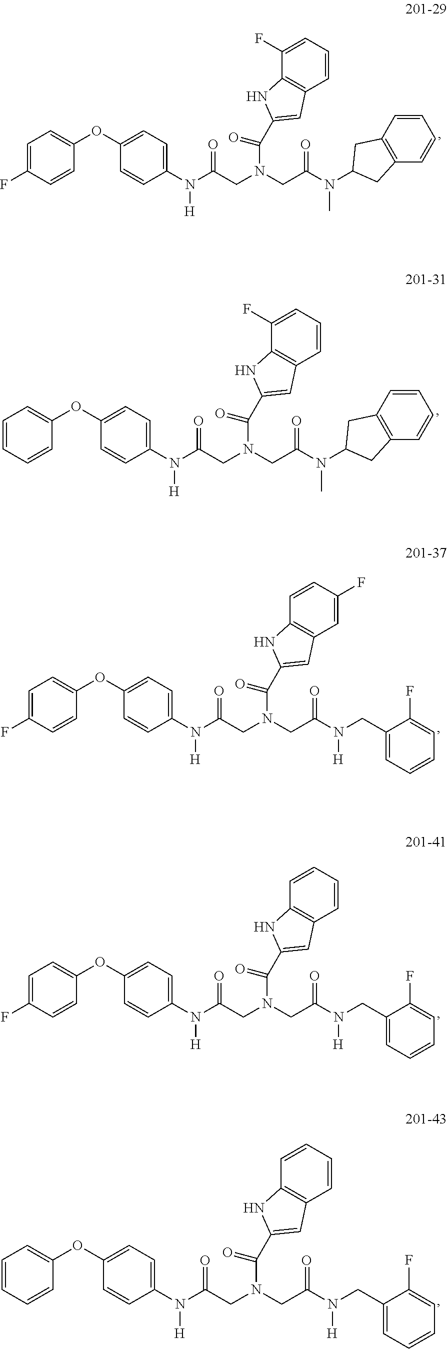 [SMILES: CN(C(=O)CN(CC(=O)Nc1ccc(Oc2ccc(F)cc2)cc1)C(=O)c1cc2cccc(F)c2[nH]1)C1Cc2ccccc2C1 ; CN(C(=O)CN(CC(=O)Nc1ccc(Oc2ccccc2)cc1)C(=O)c1cc2cccc(F)c2[nH]1)C1Cc2ccccc2C1 ; O=C(CN(CC(=O)Nc1ccc(Oc2ccc(F)cc2)cc1)C(=O)c1cc2cc(F)ccc2[nH]1)NCc1ccccc1F ; O=C(CN(CC(=O)Nc1ccc(Oc2ccc(F)cc2)cc1)C(=O)c1cc2ccccc2[nH]1)NCc1ccccc1F ; O=C(CN(CC(=O)Nc1ccc(Oc2ccccc2)cc1)C(=O)c1cc2ccccc2[nH]1)NCc1ccccc1F]

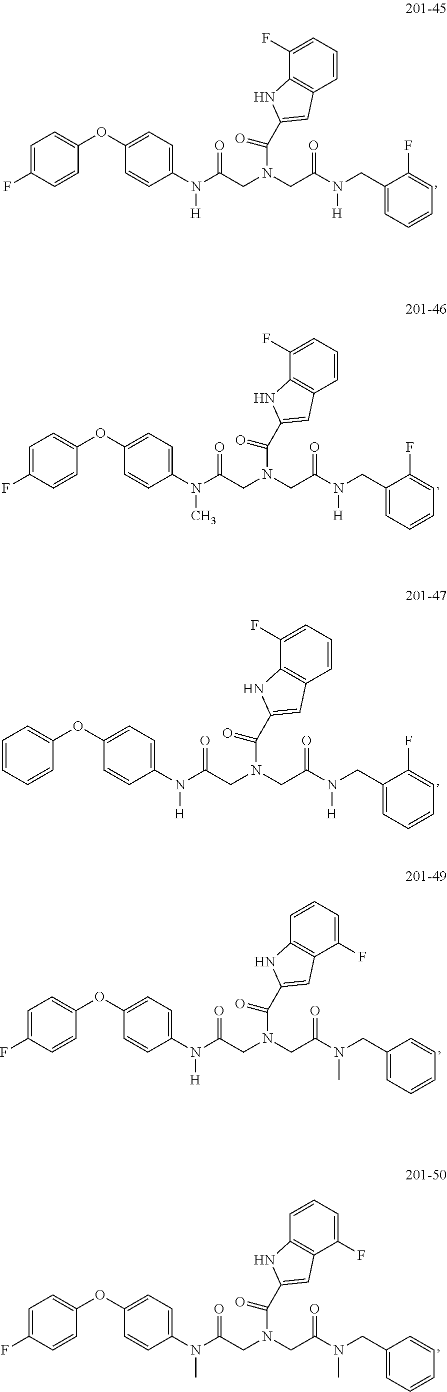 [SMILES: O=C(CN(CC(=O)Nc1ccc(Oc2ccc(F)cc2)cc1)C(=O)c1cc2cccc(F)c2[nH]1)NCc1ccccc1F ; CN(C(=O)CN(CC(=O)NCc1ccccc1F)C(=O)c1cc2cccc(F)c2[nH]1)c1ccc(Oc2ccc(F)cc2)cc1 ; O=C(CN(CC(=O)Nc1ccc(Oc2ccccc2)cc1)C(=O)c1cc2cccc(F)c2[nH]1)NCc1ccccc1F ; CN(Cc1ccccc1)C(=O)CN(CC(=O)Nc1ccc(Oc2ccc(F)cc2)cc1)C(=O)c1cc2c(F)cccc2[nH]1 ; CN(Cc1ccccc1)C(=O)CN(CC(=O)N(C)c1ccc(Oc2ccc(F)cc2)cc1)C(=O)c1cc2c(F)cccc2[nH]1]

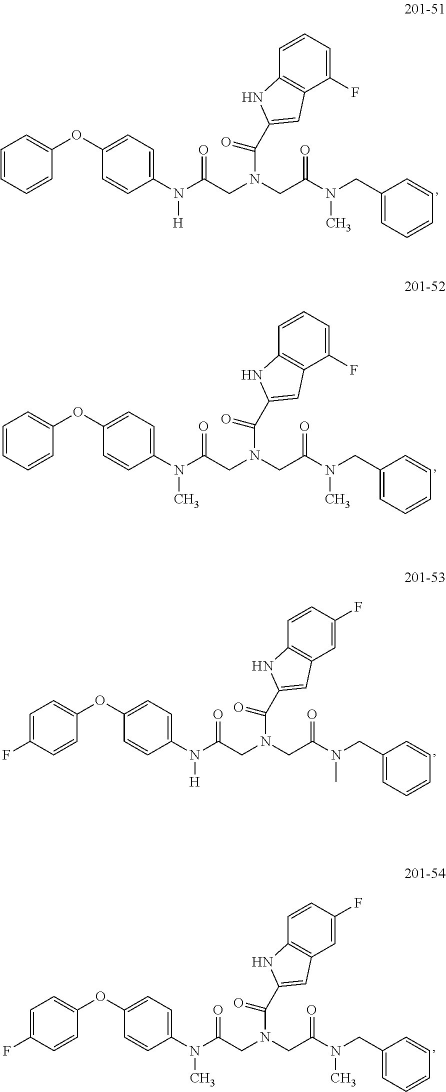 [SMILES: CN(Cc1ccccc1)C(=O)CN(CC(=O)Nc1ccc(Oc2ccccc2)cc1)C(=O)c1cc2c(F)cccc2[nH]1 ; CN(Cc1ccccc1)C(=O)CN(CC(=O)N(C)c1ccc(Oc2ccccc2)cc1)C(=O)c1cc2c(F)cccc2[nH]1 ; CN(Cc1ccccc1)C(=O)CN(CC(=O)Nc1ccc(Oc2ccc(F)cc2)cc1)C(=O)c1cc2cc(F)ccc2[nH]1 ; CN(Cc1ccccc1)C(=O)CN(CC(=O)N(C)c1ccc(Oc2ccc(F)cc2)cc1)C(=O)c1cc2cc(F)ccc2[nH]1]

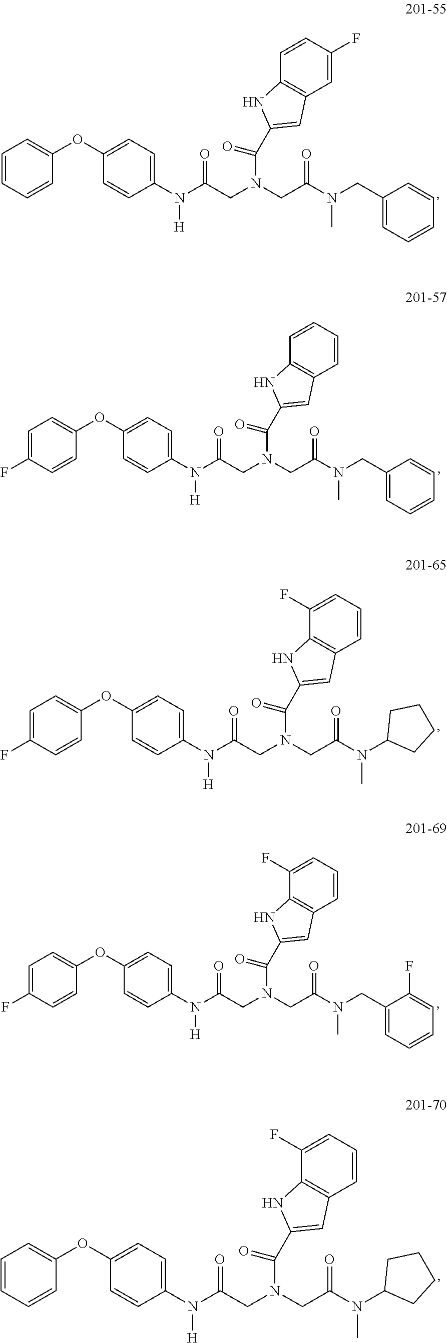 [SMILES: CN(Cc1ccccc1)C(=O)CN(CC(=O)Nc1ccc(Oc2ccccc2)cc1)C(=O)c1cc2cc(F)ccc2[nH]1 ; CN(Cc1ccccc1)C(=O)CN(CC(=O)Nc1ccc(Oc2ccc(F)cc2)cc1)C(=O)c1cc2ccccc2[nH]1 ; CN(C(=O)CN(CC(=O)Nc1ccc(Oc2ccc(F)cc2)cc1)C(=O)c1cc2cccc(F)c2[nH]1)C1CCCC1 ; CN(Cc1ccccc1F)C(=O)CN(CC(=O)Nc1ccc(Oc2ccc(F)cc2)cc1)C(=O)c1cc2cccc(F)c2[nH]1 ; CN(C(=O)CN(CC(=O)Nc1ccc(Oc2ccccc2)cc1)C(=O)c1cc2cccc(F)c2[nH]1)C1CCCC1]

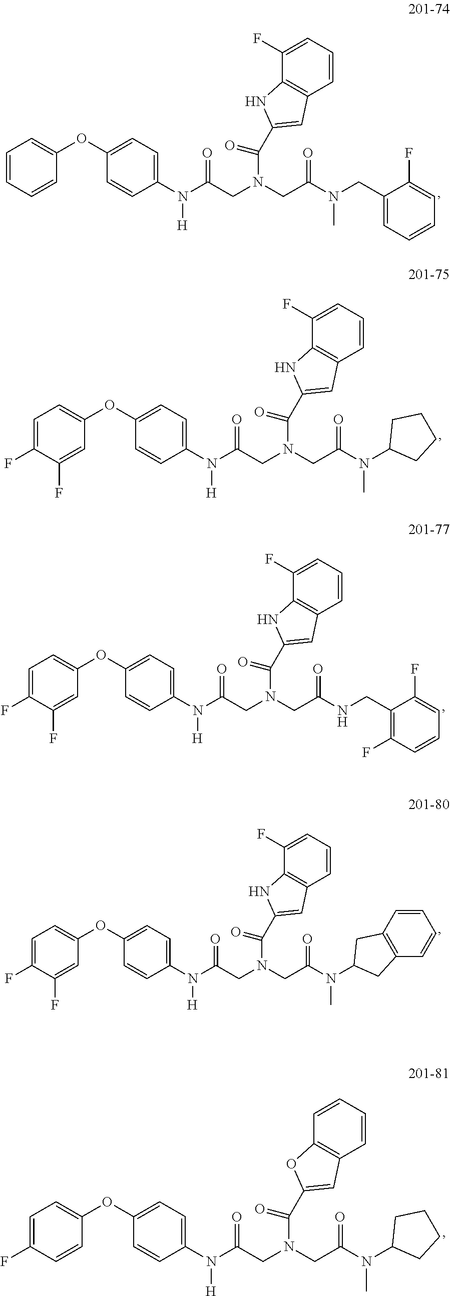 [SMILES: CN(Cc1ccccc1F)C(=O)CN(CC(=O)Nc1ccc(Oc2ccccc2)cc1)C(=O)c1cc2cccc(F)c2[nH]1 ; CN(C(=O)CN(CC(=O)Nc1ccc(Oc2ccc(F)c(F)c2)cc1)C(=O)c1cc2cccc(F)c2[nH]1)C1CCCC1 ; O=C(CN(CC(=O)Nc1ccc(Oc2ccc(F)c(F)c2)cc1)C(=O)c1cc2cccc(F)c2[nH]1)NCc1c(F)cccc1F ; CN(C(=O)CN(CC(=O)Nc1ccc(Oc2ccc(F)c(F)c2)cc1)C(=O)c1cc2cccc(F)c2[nH]1)C1Cc2ccccc2C1 ; CN(C(=O)CN(CC(=O)Nc1ccc(Oc2ccc(F)cc2)cc1)C(=O)c1cc2ccccc2o1)C1CCCC1]

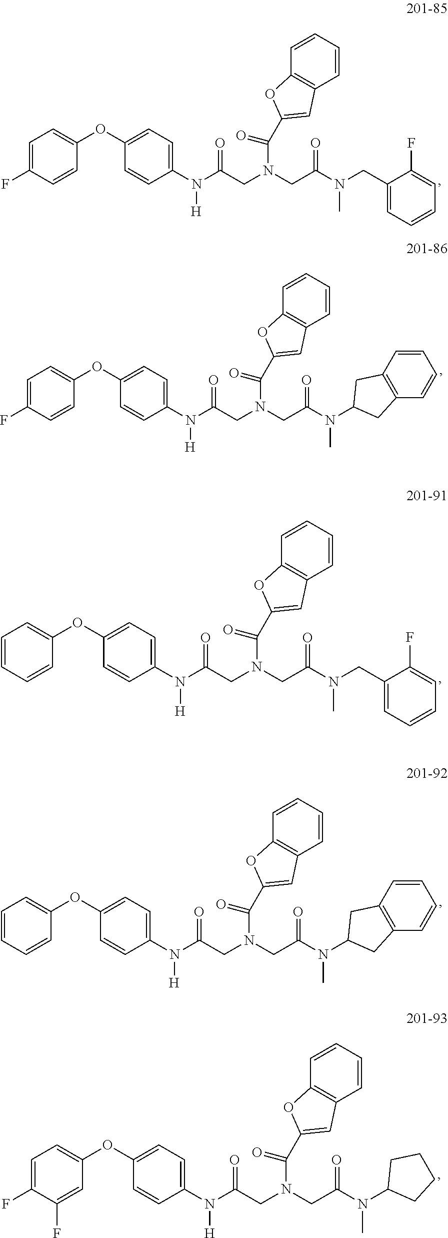 [SMILES: CN(Cc1ccccc1F)C(=O)CN(CC(=O)Nc1ccc(Oc2ccc(F)cc2)cc1)C(=O)c1cc2ccccc2o1 ; CN(C(=O)CN(CC(=O)Nc1ccc(Oc2ccc(F)cc2)cc1)C(=O)c1cc2ccccc2o1)C1Cc2ccccc2C1 ; CN(Cc1ccccc1F)C(=O)CN(CC(=O)Nc1ccc(Oc2ccccc2)cc1)C(=O)c1cc2ccccc2o1 ; CN(C(=O)CN(CC(=O)Nc1ccc(Oc2ccccc2)cc1)C(=O)c1cc2ccccc2o1)C1Cc2ccccc2C1 ; CN(C(=O)CN(CC(=O)Nc1ccc(Oc2ccc(F)c(F)c2)cc1)C(=O)c1cc2ccccc2o1)C1CCCC1]

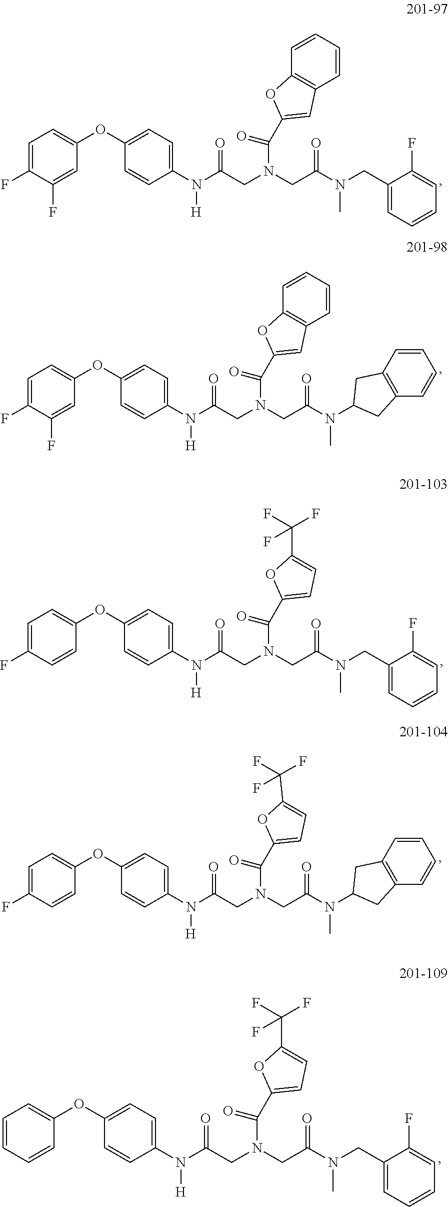 [SMILES: CN(Cc1ccccc1F)C(=O)CN(CC(=O)Nc1ccc(Oc2ccc(F)c(F)c2)cc1)C(=O)c1cc2ccccc2o1 ; CN(C(=O)CN(CC(=O)Nc1ccc(Oc2ccc(F)c(F)c2)cc1)C(=O)c1cc2ccccc2o1)C1Cc2ccccc2C1 ; CN(Cc1ccccc1F)C(=O)CN(CC(=O)Nc1ccc(Oc2ccc(F)cc2)cc1)C(=O)c1ccc(C(F)(F)F)o1 ; CN(C(=O)CN(CC(=O)Nc1ccc(Oc2ccc(F)cc2)cc1)C(=O)c1ccc(C(F)(F)F)o1)C1Cc2ccccc2C1 ; CN(Cc1ccccc1F)C(=O)CN(CC(=O)Nc1ccc(Oc2ccccc2)cc1)C(=O)c1ccc(C(F)(F)F)o1]

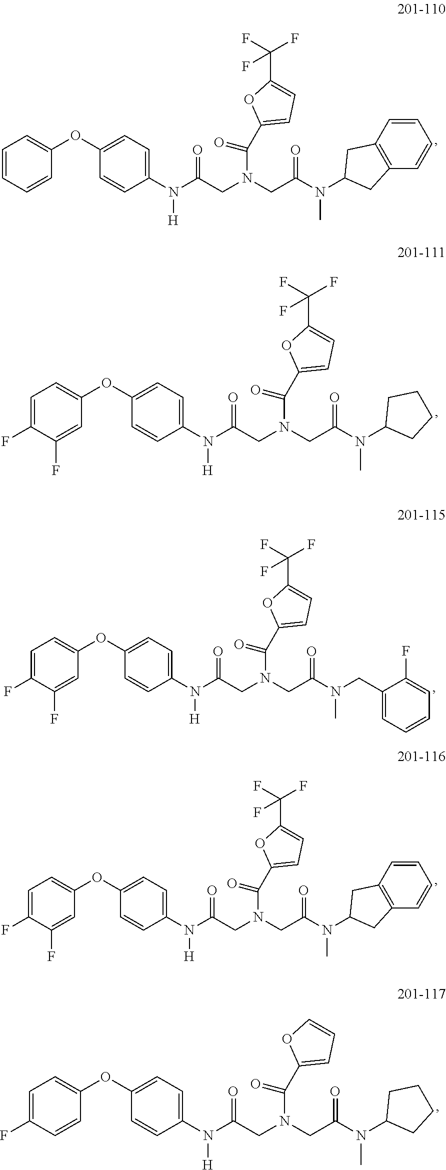 [SMILES: CN(C(=O)CN(CC(=O)Nc1ccc(Oc2ccccc2)cc1)C(=O)c1ccc(C(F)(F)F)o1)C1Cc2ccccc2C1 ; CN(C(=O)CN(CC(=O)Nc1ccc(Oc2ccc(F)c(F)c2)cc1)C(=O)c1ccc(C(F)(F)F)o1)C1CCCC1 ; CN(Cc1ccccc1F)C(=O)CN(CC(=O)Nc1ccc(Oc2ccc(F)c(F)c2)cc1)C(=O)c1ccc(C(F)(F)F)o1 ; CN(C(=O)CN(CC(=O)Nc1ccc(Oc2ccc(F)c(F)c2)cc1)C(=O)c1ccc(C(F)(F)F)o1)C1Cc2ccccc2C1 ; CN(C(=O)CN(CC(=O)Nc1ccc(Oc2ccc(F)cc2)cc1)C(=O)c1ccco1)C1CCCC1]

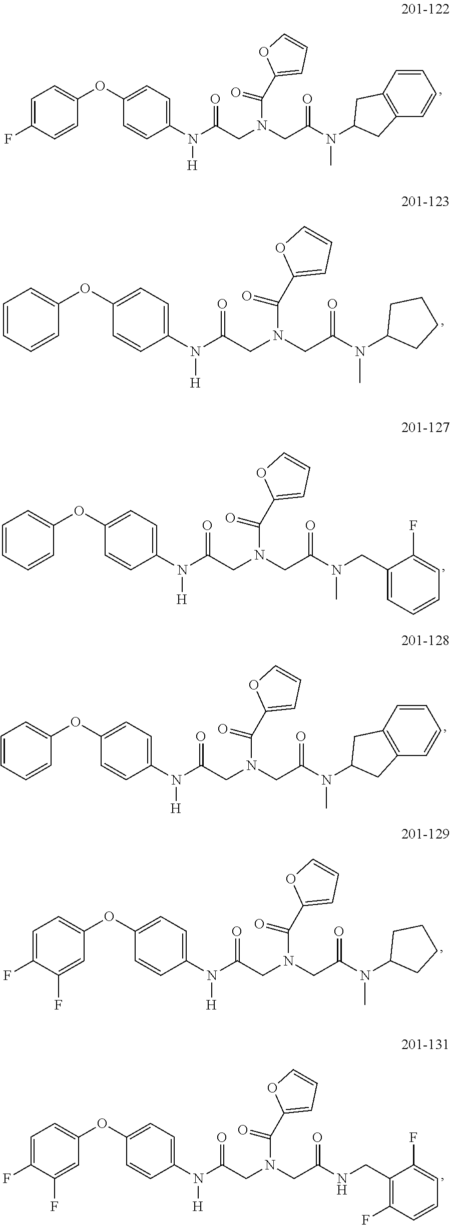 [SMILES: CN(C(=O)CN(CC(=O)Nc1ccc(Oc2ccc(F)cc2)cc1)C(=O)c1ccco1)C1Cc2ccccc2C1 ; CN(C(=O)CN(CC(=O)Nc1ccc(Oc2ccccc2)cc1)C(=O)c1ccco1)C1CCCC1 ; CN(Cc1ccccc1F)C(=O)CN(CC(=O)Nc1ccc(Oc2ccccc2)cc1)C(=O)c1ccco1 ; CN(C(=O)CN(CC(=O)Nc1ccc(Oc2ccccc2)cc1)C(=O)c1ccco1)C1Cc2ccccc2C1 ; CN(C(=O)CN(CC(=O)Nc1ccc(Oc2ccc(F)c(F)c2)cc1)C(=O)c1ccco1)C1CCCC1 ; O=C(CN(CC(=O)Nc1ccc(Oc2ccc(F)c(F)c2)cc1)C(=O)c1ccco1)NCc1c(F)cccc1F]

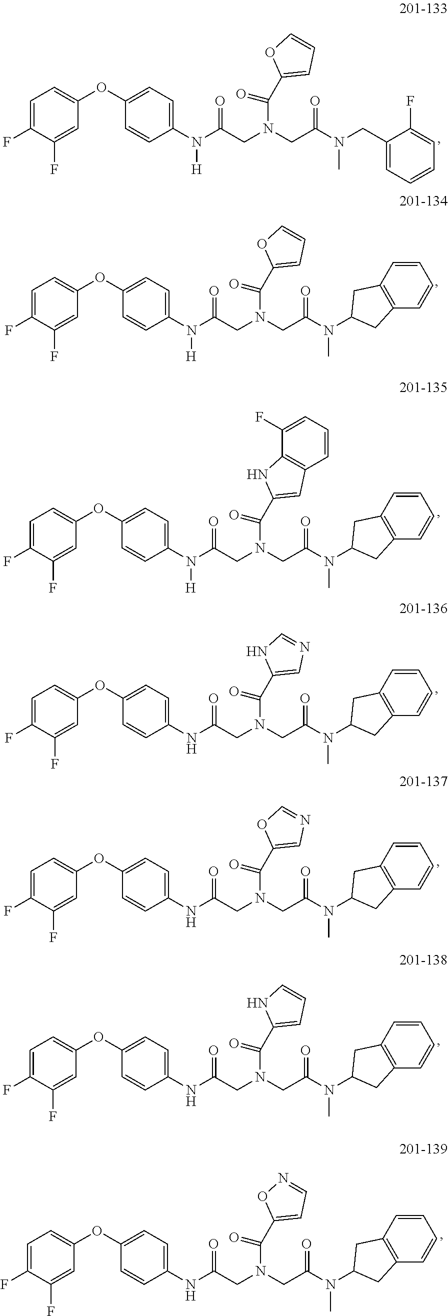

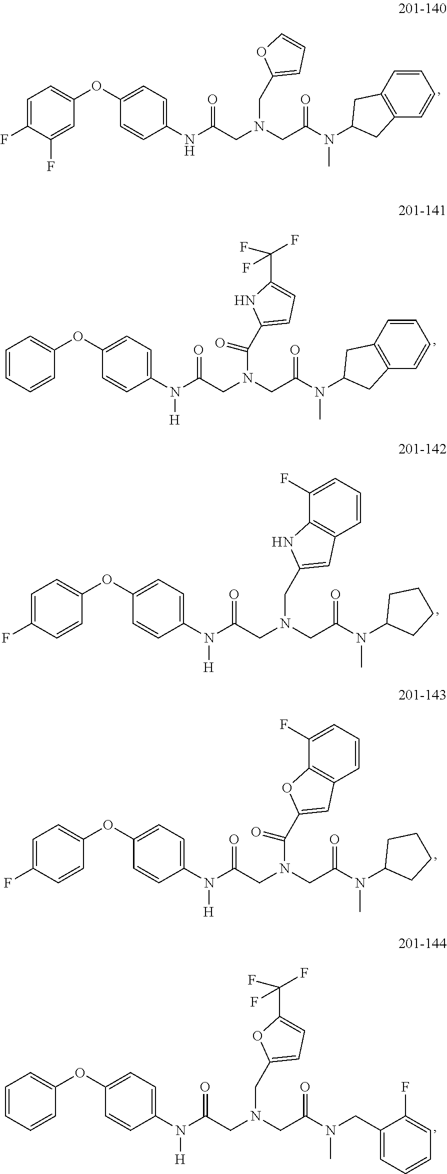 [SMILES: CN(C(=O)CN(CC(=O)Nc1ccc(Oc2ccc(F)c(F)c2)cc1)Cc1ccco1)C1Cc2ccccc2C1 ; CN(C(=O)CN(CC(=O)Nc1ccc(Oc2ccccc2)cc1)C(=O)c1ccc(C(F)(F)F)[nH]1)C1Cc2ccccc2C1 ; CN(C(=O)CN(CC(=O)Nc1ccc(Oc2ccc(F)cc2)cc1)Cc1cc2cccc(F)c2[nH]1)C1CCCC1 ; CN(C(=O)CN(CC(=O)Nc1ccc(Oc2ccc(F)cc2)cc1)C(=O)c1cc2cccc(F)c2o1)C1CCCC1 ; CN(Cc1ccccc1F)C(=O)CN(CC(=O)Nc1ccc(Oc2ccccc2)cc1)Cc1ccc(C(F)(F)F)o1]

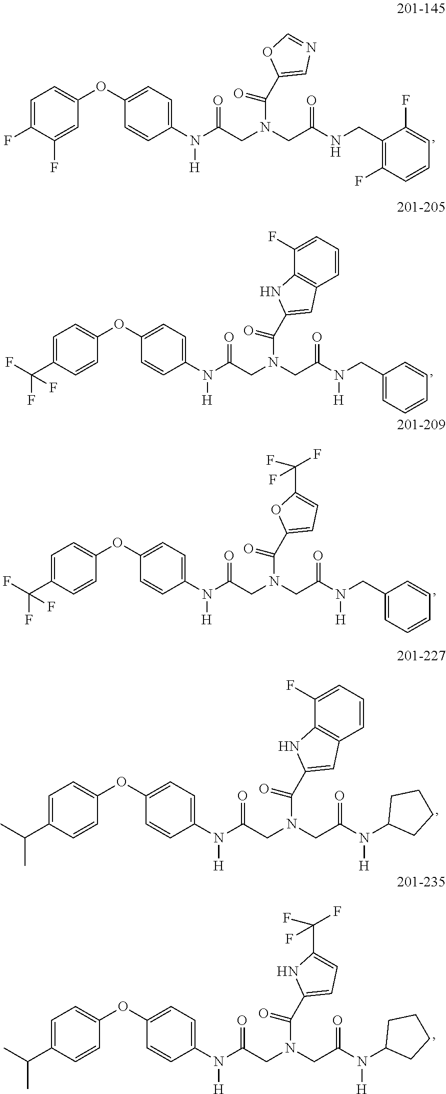 [SMILES: O=C(CN(CC(=O)Nc1ccc(Oc2ccc(F)c(F)c2)cc1)C(=O)c1cnco1)NCc1c(F)cccc1F ; O=C(CN(CC(=O)Nc1ccc(Oc2ccc(C(F)(F)F)cc2)cc1)C(=O)c1cc2cccc(F)c2[nH]1)NCc1ccccc1 ; O=C(CN(CC(=O)Nc1ccc(Oc2ccc(C(F)(F)F)cc2)cc1)C(=O)c1ccc(C(F)(F)F)o1)NCc1ccccc1 ; CC(C)c1ccc(Oc2ccc(NC(=O)CN(CC(=O)NC3CCCC3)C(=O)c3cc4cccc(F)c4[nH]3)cc2)cc1 ; CC(C)c1ccc(Oc2ccc(NC(=O)CN(CC(=O)NC3CCCC3)C(=O)c3ccc(C(F)(F)F)[nH]3)cc2)cc1]

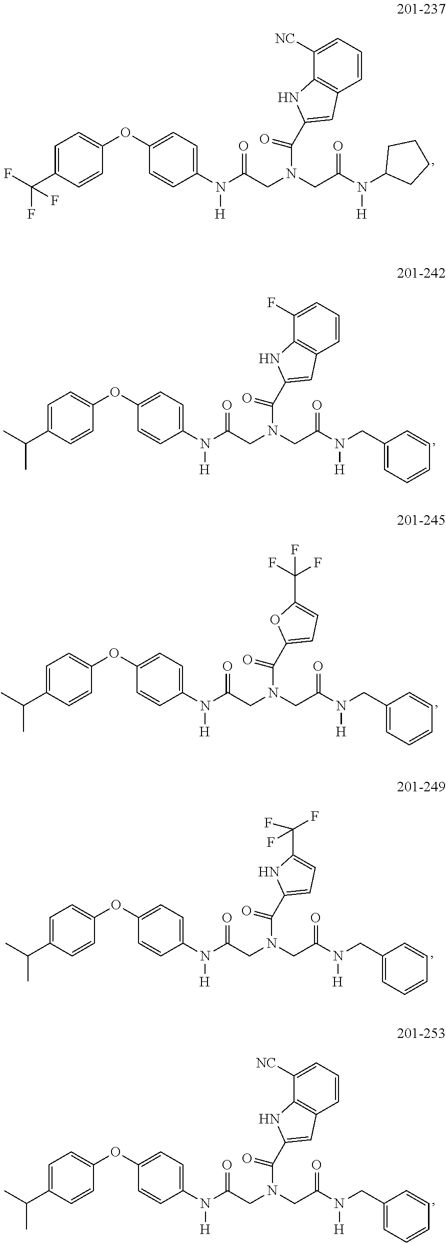 [SMILES: N#Cc1cccc2cc(C(=O)N(CC(=O)Nc3ccc(Oc4ccc(C(F)(F)F)cc4)cc3)CC(=O)NC3CCCC3)[nH]c12 ; CC(C)c1ccc(Oc2ccc(NC(=O)CN(CC(=O)NCc3ccccc3)C(=O)c3cc4cccc(F)c4[nH]3)cc2)cc1 ; CC(C)c1ccc(Oc2ccc(NC(=O)CN(CC(=O)NCc3ccccc3)C(=O)c3ccc(C(F)(F)F)o3)cc2)cc1 ; CC(C)c1ccc(Oc2ccc(NC(=O)CN(CC(=O)NCc3ccccc3)C(=O)c3ccc(C(F)(F)F)[nH]3)cc2)cc1 ; CC(C)c1ccc(Oc2ccc(NC(=O)CN(CC(=O)NCc3ccccc3)C(=O)c3cc4cccc(C#N)c4[nH]3)cc2)cc1]

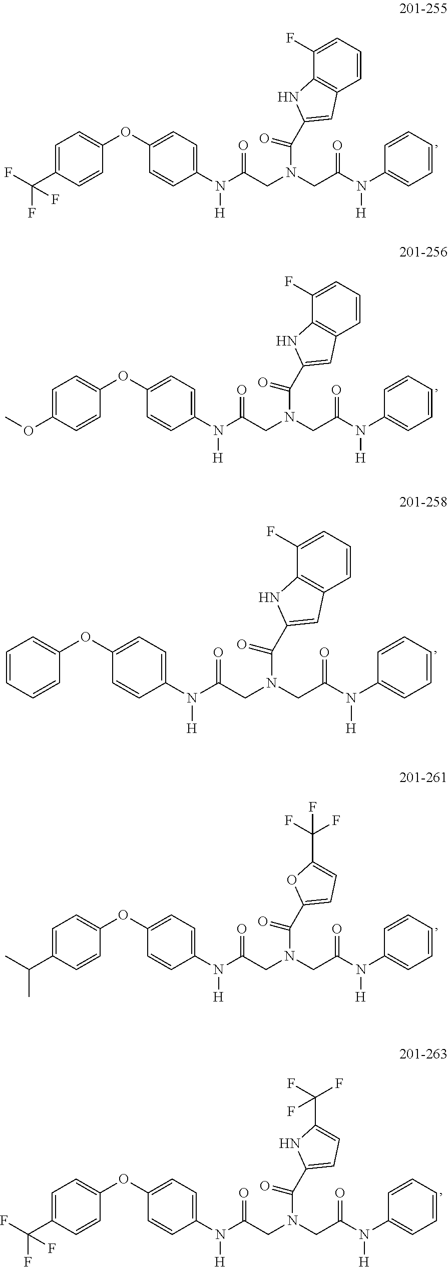 [SMILES: O=C(CN(CC(=O)Nc1ccc(Oc2ccc(C(F)(F)F)cc2)cc1)C(=O)c1cc2cccc(F)c2[nH]1)Nc1ccccc1 ; COc1ccc(Oc2ccc(NC(=O)CN(CC(=O)Nc3ccccc3)C(=O)c3cc4cccc(F)c4[nH]3)cc2)cc1 ; O=C(CN(CC(=O)Nc1ccc(Oc2ccccc2)cc1)C(=O)c1cc2cccc(F)c2[nH]1)Nc1ccccc1 ; CC(C)c1ccc(Oc2ccc(NC(=O)CN(CC(=O)Nc3ccccc3)C(=O)c3ccc(C(F)(F)F)o3)cc2)cc1 ; O=C(CN(CC(=O)Nc1ccc(Oc2ccc(C(F)(F)F)cc2)cc1)C(=O)c1ccc(C(F)(F)F)[nH]1)Nc1ccccc1]

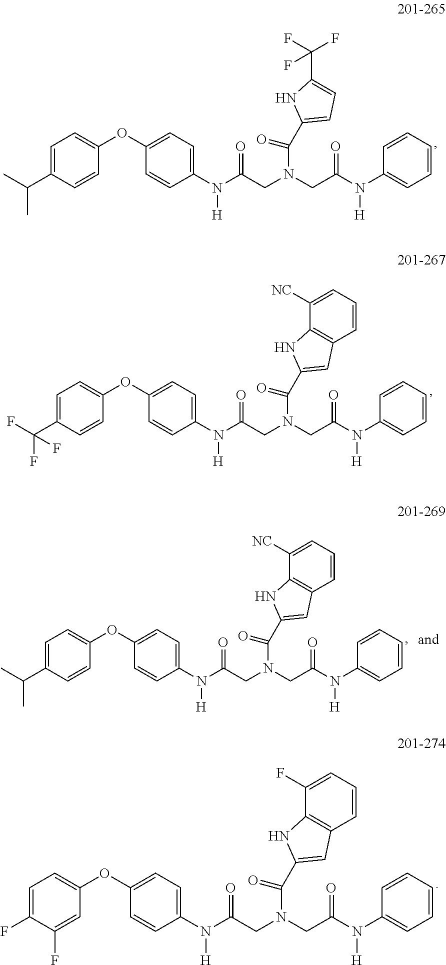 [SMILES: CC(C)c1ccc(Oc2ccc(NC(=O)CN(CC(=O)Nc3ccccc3)C(=O)c3ccc(C(F)(F)F)[nH]3)cc2)cc1 ; N#Cc1cccc2cc(C(=O)N(CC(=O)Nc3ccccc3)CC(=O)Nc3ccc(Oc4ccc(C(F)(F)F)cc4)cc3)[nH]c12 ; CC(C)c1ccc(Oc2ccc(NC(=O)CN(CC(=O)Nc3ccccc3)C(=O)c3cc4cccc(C#N)c4[nH]3)cc2)cc1 ; O=C(CN(CC(=O)Nc1ccc(Oc2ccc(F)c(F)c2)cc1)C(=O)c1cc2cccc(F)c2[nH]1)Nc1ccccc1]

21. A pharmaceutical composition, comprising a compound according to claim 1, or a pharmaceutically acceptable salt, an enantiomer, a diastereomer, a racemate, a solvate, a hydrate, a polymorph, a prodrug or a isotope variant thereof, and pharmaceutically acceptable excipients; optionally, the pharmaceutical composition further comprises one or more other therapeutic agents.

22. (canceled)

23. A method of treating and/or preventing a PCSK9-mediated disease in a subject, comprising administering to the subject a compound according to claim 1, or a pharmaceutically acceptable salt, an enantiomer, a diastereomer, a racemate, a solvate, a hydrate, a polymorph, a prodrug or a isotope variant thereof.

24. (canceled)

25. The method of claim 23, wherein the PCSK9-mediated disease is selected from atherosclerosis, dyslipidemia, hypertriglyceridemia, hypertension, heart failure, cardiac arrhythmias, low HDL levels, high LDL levels, sudden death, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia (including heterozygous and homozygous familial hypercholesterolemia), stroke, hyperlipidemia, hyperlipoproteinemia, chronic kidney disease, intermittent claudication, hyperphosphatemia, carotid atherosclerosis, peripheral arterial disease, diabetic nephropathy, hypercholesterolemia in HIV infection, acute coronary syndrome (ACS), non-alcoholic fatty liver disease, arterial occlusive diseases, cerebral arteriosclerosis, cerebrovascular disorders, myocardial ischemia, nonalcoholic fatty liver disease (NLLD), nonalcoholic steatohepatitis (NASH), and diabetic autonomic neuropathy.
